(19) United States
(12) Patent Application Publication
(10) Pub. No.: US 2004/0248299 A1
(43) Pub. Date: Dec. 9, 2004

(54) RNA interference

(76) Inventors: Sumedha Jayasena, Thousand Oaks, CA (US); Angela Reynolds, Littleton, CO (US); Anastasia Khvorova, Boulder Creek, CO (US)

Correspondence Address: Finnergan, Henderson, Farabow,, Garrett & Dunner, L.L.P., 1300 I Street, N.W., Washington, DC, 20005-3315, US

(21) Appl. No.: 10/745,395

(22) Filed: Dec. 22, 2003

Related U.S. Application Data
(60) Provisional application No. 60/436,849, filed on Dec. 27, 2002.

(51) Int. Cl. C12N 15/85

(52) U.S. Cl. 435/455

(57) ABSTRACT

The present invention relates to RNA interference and methods for selecting interfering RNAs. The present invention also relates to modified interfering RNAs. The present invention also relates to methods of reducing the level of a specific mRNA in a cell, methods for reducing the level of a specific protein in a cell, and methods of regulating gene expression. The present invention also relates to methods of screening libraries for an interfering RNA of interest and methods of screening libraries based on a gene function.

### PRIORITY INFORMATION

This application claims priority benefit of U.S. Patent Application No. 60/436,849, filed Dec. 27, 2002. The entire contents of U.S. Patent Application No. 60/436,849 is specifically incorporated herein by reference in its entirety.

### FIELD OF THE INVENTION

The present invention relates to RNA interference. The present invention also relates to methods of selecting interfering RNAs. The present invention also relates to methods of using interfering RNAs. The present invention also relates to RNA interference based assays to identify target genes.

### BACKGROUND OF THE INVENTION

RNA interference is a process by which specific mRNAs are degraded into short RNAs. RNA interference has been observed in organisms as diverse as nematodes, insects, trypanosomes, planaria, hydra, zebrafish, and mice. To mediate RNA interference, a double-stranded RNA with substantial sequence identity to the target mRNA is introduced into a cell. The target mRNA is then degraded in the cell, resulting in decreased levels of that mRNA and the protein it encodes.

Many different mRNAs have been targeted in this manner. Various labs have demonstrated that RNA interference is functional in vitro, e.g., in Drosophila extracts, and in vivo, e.g., in C. elegans.

### SUMMARY OF THE INVENTION

In certain embodiments, a method of decreasing the level of a target mRNA in a host cell is provided. In certain embodiments, a host cell is contacted with a double-stranded RNA molecule, wherein the double-stranded RNA molecule comprises a sequence complementary to at least a portion of the target mRNA. In certain embodiments, the double-stranded RNA molecule further comprises at least one chemical modification. In certain embodiments, the at least one chemical modification is selected from 2′-F, 2′-OMe, and 2′-deoxy. In certain embodiments, the host cell is incubated under conditions whereby RNA interference occurs, thereby decreasing the level of the target mRNA.

In certain embodiments, a method of decreasing the level of a target mRNA in a host cell is provided. In certain embodiments, a vector is delivered to the host cell. In certain embodiments, the vector comprises a first nucleic acid sequence and a second nucleic acid sequence. In certain embodiments, the first nucleic acid sequence encodes a first RNA molecule comprising a first RNA sequence that is complementary to at least a portion of the target mRNA. In certain embodiments, the second nucleic acid sequence encodes a second RNA molecule comprising a second RNA sequence that is substantially identical to at least a portion of the target mRNA. In certain embodiments, the host cell is incubated under conditions that allow transcription of the first nucleic acid sequence and the second nucleic acid sequence. In certain embodiments, the host cell is incubated under conditions that allow RNA interference to occur, thereby decreasing the level of the target mRNA.

In certain embodiments, the first RNA sequence and the second RNA sequence are each longer than about 70 nucleotides. In certain embodiments, the vector further comprises at least one promoter selected from a phage promoter, a viral promoter, a pol II promoter, and a pol III promoter.

In certain embodiments, a method of selecting a double-stranded RNA molecule is provided. In certain embodiments, a target mRNA sequence is inputted into Oligo 5.0™ Primer Analysis software. In certain embodiments, the primer length is selected as 19. In certain embodiments, a primer is identified in the stability window, wherein the primer has a bell-shaped internal energy profile. In certain embodiments, a primer is identified in the stability window, wherein the primer has a substantially flat internal energy profile. In certain embodiments, a primer is identified in the stability window, wherein the primer has a maximum internal energy of less than −10 kcal/mol. In certain embodiments, a primer is identified in the stability window, wherein the primer has an internal energy of between −6 and −9 kcal/mol. In certain embodiments, a primer is identified in the stability window, wherein the primer has a melting temperature below 65° C. In certain embodiments, a primer is identified in the stability window, wherein the primer has a melting temperature below 50° C.

In certain embodiments, a BLAST search is performed on the primer against an EST database. In certain embodiments, a double-stranded RNA is synthesized, wherein the double-stranded RNA comprises a first RNA strand comprising a first RNA sequence that is identical to the nucleotide sequence of the primer and a second RNA strand comprising a second RNA sequence that is complementary to the nucleotide sequence of the primer.

In certain embodiments, a method of decreasing the level of a target mRNA in a mammalian host cell is provided. In certain embodiments, a mammalian host cell is contacted with an RNA hairpin molecule. In certain embodiments, the RNA hairpin molecule comprises a first region, a second region, and a third region. In certain embodiments, the first region comprises a sequence that is substantially identical to at least a portion of the target mRNA. In certain embodiments, the third region comprises a sequence that is substantially complementary to the first region. In certain embodiments, the first region and the third region hybridize, thereby forming an RNA hairpin molecule. In certain embodiments, the mammalian host cell is incubated under conditions whereby RNA interference occurs, thereby decreasing the level of the target mRNA in the host cell.

In certain embodiments, a method of decreasing the level of a target mRNA in a host cell is provided. In certain embodiments, a vector is delivered to the host cell. In certain embodiments, the vector comprises a nucleic acid sequence, wherein the nucleic acid sequence encodes an RNA hairpin molecule. In certain embodiments, the RNA hairpin molecule comprises a first region, a second region, and a third region. In certain embodiments, the first region comprises a sequence that is substantially identical to at least a portion of the target mRNA. In certain embodiments, the third region comprises a sequence that is substantially complementary to the first region. In certain embodiments, the first region and the third region hybridize, thereby forming an RNA hairpin molecule. In certain embodiments, the host cell is incubated under conditions that allow transcription of the nucleic acid sequence. In certain embodiments, the host cell is incubated under conditions that allow RNA interference to occur, thereby decreasing the level of the target mRNA in the host cell.

In certain embodiments, a method of constructing a library of RNA hairpin molecules is provided. In certain embodiments, a plurality of single-stranded DNA hairpin templates is synthesized. In certain embodiments, each single-stranded DNA hairpin template comprises a first region, a second region, and a third region. In certain embodiments, the first region comprises an RNA polymerase promoter sequence. In certain embodiments, the second region comprises a random nucleotide sequence having between 5 and 500 nucleotides. In certain embodiments, the third region comprises a first nucleotide sequence, a second nucleotide sequence, and a third nucleotide sequence, wherein the first nucleotide sequence hybridizes to the third nucleotide sequence, thereby forming a single-stranded DNA hairpin template. In certain embodiments, the 3′ end of the third nucleotide sequence of each of the plurality of single-stranded DNA hairpin templates is extended to form a plurality of double-stranded DNA hairpin templates. In certain embodiments, the plurality of double-stranded DNA hairpin templates is amplified to form a plurality of double-stranded DNA templates. In certain embodiments, the plurality of double-stranded DNA templates is transcribed to form a library of RNA hairpin molecules.

In certain embodiments, a method of identifying a target gene is provided. In certain embodiments, an array comprising a plurality of positions is formed. In certain embodiments, each position comprises at least one mammalian cell. In certain embodiments, the at least one mammalian cell at each position is contacted with at least one RNA hairpin molecule. In certain embodiments, the at least one mammalian cell is incubated under conditions that allow RNA interference to occur. In certain embodiments, an at least one mammalian cell exhibiting at least one biological endpoint is selected. In certain embodiments, the at least one RNA hairpin molecule associated with the plurality of cells exhibiting at least one biological endpoint is identified. A BLAST search on the nucleic acid sequence of the at least one RNA hairpin molecule is performed, thereby identifying the target gene.

In certain embodiments, a library comprising a plurality of RNA hairpin molecules is provided. In certain embodiments, each RNA hairpin molecule comprises a first region, a second region, and a third region, wherein the first region comprises a random nucleotide sequence having between 5 and 500 nucleotides and the third region comprises a nucleotide sequence that is substantially complementary to the first region.

### BRIEF DESCRIPTION OF THE DRAWINGS

FIG. 1 shows a schematic representation of certain proposed mechanisms of RNA interference. Double-stranded RNA (dsRNA) is cleaved into 21-25 nucleotide fragments by an endonuclease. The sense strands of the dsRNA hybridize to the target mRNA, and the hybridized target mRNA is cleaved into 21-25 nucleotide fragments by an endonuclease. Those fragments may then hybridize to another copy of the target mRNA, leading to its cleavage by endonuclease.

FIG. 2 shows a schematic representation of certain assays for identifying modified dsRNAs that are active in RNA interference. (A) A chemically-modified dsRNA (A) or a hairpin or circular dsRNA (B) having a sequence identical to a portion of a luciferase gene is incubated with a luciferase reporter gene expression construct in a Drosophila extract. Luciferase expression in the extract is then detected. A decrease in luciferase expression indicates a functional modified dsRNA.

FIG. 3 shows a schematic representation of the in vivo expression of dsRNA for RNA interference according to certain embodiments. (A) A DNA expression construct having a sequence encoding the dsRNA sense strand and a sequence encoding the dsRNA antisense strand is transformed or transfected into a host cell. The DNA expression construct expresses the sense and antisense strands, which associate in the cell to form a dsRNA. The dsRNA is cleaved into 21-25 dsRNA fragments by endonuclease. The fragments hybridize to the target mRNA, causing it to be cleaved into short fragments as well. (B) A DNA expression construct having several sequences that encode a hairpin RNA is transformed or transfected into a host cell. The DNA expression construct expresses the hairpin RNA, which is then cleaved by endonuclease to form dsRNA fragments. The fragments hybridize to the target mRNA, causing it to be cleaved into short fragments as well.

FIG. 4 shows the ds-SiRNA and antisense oligonucleotides used in a study (SEQ ID NOS: 1 to 15). Antisense nucleic acid sequences are 25-nucleotide-long ss-DNAs in which the middle seven nucleotides carry a 2′-Omethyl modification and the remaining nucleotides on either side have a phosphorothioate backbone modification. These sequences target three independent sites (#4, #8 and #15) on the PKC-θ mRNA that were picked using a computer program designed to identify the best sites within an mRNA sequence for antisense targeting. The ds-SiRNA molecules are based on the three antisense sequences, except that they have either 19- or 21 nucleotide target homology instead of 25 nucleotide homology as in antisense DNA. The ds-SiRNA molecules have 3′ extensions with two deoxythymidines (dTs) that do not base pair with the target mRNA. For each target site, two ds-RNAs, one with a 21 nucleotide (SiRNA-A) and the other with a 19 nucleotide (SiRNA-B) target homology, were chemically synthesized. In a SiRNA molecule the top strand indicates the antisense strand, whereas the bottom strand is the sense strand.

FIG. 5 shows sequences and the predicted secondary structures of ss-SiRNA triggers used in a study (SEQ ID NOS. 16 to 21). For each target site, two ss-SiRNA triggers were designed with the propensity to form short hairpins. Out of the two molecules, one (SiRNA-HP) with six dT nucleotides in the loop and two dT residues in the 3′-extension was chemically synthesized. The other (SiRNA-HP-T) ss-SiRNA molecule containing six uridines in the loop and two uridines at the 3′ recessed end was obtained by in vitro transcription using corresponding synthetic DNA templates. SiRNA-#8-HP-T that contains a single nucleotide deletion within the region of homology was used as a control.

FIG. 6 shows reduction of target mRNA by defined short hairpin RNAi triggers in cultured mammalian cells. This work targeted the PKC-θ gene, a gene that is expressed endogenously in NIH 293 cells (a human kidney cell line) by a range of nucleic acid triggers. These triggers include ds-SiRNA, ss-SiRNA with the propensity to form short hairpins, and antisense DNA oligonucleotides. Sequences and secondary structures of triggers are illustrated in FIGS. 4 and 5.

Various nucleic acid triggers were delivered into NIH 293 cells grown in 96-well plates by transfection with Lipofectamin-2000. Twenty-four hours after the transfection, levels of two specific messages, PKC-θ and Cyclophilin, were quantified by branched-DNA (bDNA) capture detection method. Cyclophilin, a housekeeping gene, was used to detect any nonspecific gene inhibition induced by various nucleic acid triggers. What is shown is the ratio of the mRNA levels of the two genes as a function of various triggers. Arrows indicate the reduction of the ratio triggered by synthetic and transcribed hairpin RNA molecules. The two triangles indicate the lack of gene knockdown by the ss-SiRNA hairpin containing a single nucleotide deletion, indicating that the RNA interference process is selective and uses hairpin triggers containing specific sequences for effective gene silencing.

FIG. 7 shows the architecture and synthesis of SiRS RNA hairpin libraries according to certain embodiments. The random sequence hairpin RNA library is obtained by in vitro transcription of a chemically synthesized DNA template whose general structure is illustrated in the inset. In this design of the random sequence DNA template, the 5′ end carries a biotin moiety for solid phase capture onto a streptavidin-coated microtiter plate. A contiguous randomized region having 15-20 nucleotides is flanked by the top strand of T7 RNA polymerase promoter and a defined nucleotide stretch that forms the closing stem to make the single-stranded DNA hairpin template. As illustrated, the 3′ end of the single-stranded DNA hairpin template is extended to complete the hairpin, creating a double-stranded DNA hairpin template, using a high fidelity DNA polymerase. Upon streptavidin capture, the double-stranded DNA hairpin template is briefly amplified by 2 cycles of PCR to obtain a double-stranded DNA template for in vitro transcription. Alternatively, the double-stranded DNA hairpin template is directly used for transcription without brief amplification to obtain a SiRS hairpin library.

FIG. 8 shows the making of “Master Plates” and “Lead Plates” using a SiRS RNA hairpin library according to certain embodiments. An initial DNA library having approximately 10⁶ individual sequences is distributed into a number of 1,536-well “Master Plates”. Although the number of “Master Plates” can vary, one may use 5 such plates to obtain approximately 400 unique template molecules/well. After 2-cycles of PCR amplification with a biotinylated primer, the resulting DNA in a single “Master Plate” is distributed into four 384-well “Lead Plates” in which each well has approximately 400 unique template molecules/well.

FIG. 9 is an illustration of a “Primary Screen” with a SiRS RNA hairpin library according to certain embodiments. A SiRS RNA hairpin library is generated within “Lead Plates” by in vitro transcription. Twenty such plates are used to cover the sequence space of the initial random library having approximately 10⁶ individual molecules. After in vitro transcription, RNA from each “Lead Plate” is transferred to a new plate (“RNA-Lead Plate”), from which SiRS RNA hairpins are delivered to cells for a number of functional assays that are carried out in parallel.

FIG. 10 shows the identification of lead SiRS hairpin libraries from a primary biological screen according to certain embodiments. Microtiter wells that produce biological end-points are identified in each assay. The SiRS RNA hairpin lead sub-libraries that produce biological endpoints in each functional assay are traced back to the “Lead Plates” to identify DNA templates from which the SiRS RNA hairpin lead sub-libraries are derived. These DNA templates that produce the functional SiRS RNA hairpin triggers (lead sub-libraries) are amplified by 2 cycles of PCR using a biotinylated primer. Resulting PCR products are distributed into several (about three) 384-well microtiter plates (“Daughter Plates”) coated with SA.

FIG. 11 shows the “Secondary Screen” with lead SiRS RNA hairpin libraries according to certain embodiments. SiRS RNA hairpin libraries produced within “Daughter Plates” are used for secondary screening. Secondary screening gives rise to candidate SiRS hairpin molecules. Finally, wells that contained DNA templates that produce candidate SiRS hairpin molecules are identified, amplified by PCR, cloned and sequenced. A consensus sequence within these candidate sequences is identified.

FIG. 12 shows the use of the consensus SiRNA hairpin triggers in identifying candidate gene(s) according to certain embodiments. The consensus sequences that are identified within the candidate SiRNA hairpin molecules are used to perform BLAST searches within the Human Genome Data Base to track the gene responsible for the function tested in the assay.

FIG. 13 shows the construction of a SiRS hairpin library for generating a retroviral library according to certain embodiments. The design of the synthetic ssDNA containing the random region with 15-20 positions is shown in the inset. Two fixed regions that provide binding sites for PCR primers flank the random region. A unique restriction site, preferably a six-base pair cutter, is included near the end of each primer to facilitate the construction of inverted repeats. Upon PCR amplification, the resulting duplex DNA is digested with one restriction site to obtain sticky ends for self-ligation. The ligated product containing inverted repeats of the random region is digested with the second restriction enzyme and is inserted into the retroviral vector.

FIG. 14 shows production of a retroviral vector harboring a SiRS hairpin library according to certain embodiments. About 10-20 μg of synthetic insert containing inverted repeats of a random region is ligated downstream of a human tRNA promoter in the viral vector. The resulting plasmids are transformed into a compatible strain of bacteria and the transformants are isolated by appropriate selection methods. Transformants are pooled and expanded, and the recombinant plasmids are purified. Finally, a retroviral library is produced in a suitable cell line using a triple transfection approach.

FIG. 15 shows a method for a biological screen with a retroviral vector expressing a SiRS hairpin library according to certain embodiments. Cells that provide a biological endpoint of interest are transduced with a retroviral vector expressing a SiRS hairpin library at a low MOI in microtiter plates. Subsequently, wells that produce a desirable phenotypic response are identified and the viruses harboring the ss-SiRNA triggers that produce the phenotypic response are rescued. After viral rescue, plasmids carrying the selected SiRS library are prepared and used for the production of a new batch of retrovirus, which are used for the next round of biological screening followed by selection. After several rounds of selection and amplification, specific hairpin sequences that produce the desired phenotype are identified by sequencing. A consensus sequence within the enriched hairpin sequences is used to perform a blast search within the human genome database to identify the candidate gene.

FIG. 16 shows the calculated average internal stability profiles and average melting temperatures (Tₘ) of certain mRNA duplexes identified in C. elegans, D. melanogaster and H. sapiens (Largos-Quintana et al., 2001, Lau et al., 2001; Lee and Ambros, 2001). Both of those parameters were calculated using Oligo 5.0™ Primer Analysis Software available from National Biosciences, Inc., Plymouth, Minn. (A) Calculated average internal stability profiles for antisense strands of mRNAs in their duplex form. For each category, a collection of individual mRNAs were used; 15 for C. elegans in Category I (Lee and Ambros, 2001), 17 for D. melanogaster, 31 for H. sapiens (Largos-Quintana et al., 2001) and 50 for C. elegans in Category IV (Lau et al., 2001). For each collection of 19-basepair miRNA duplexes, average internal stability of internal 17 nucleotide positions was calculated by averaging values of the same position in individual mRNAs. (B) Comparison between calculated average internal stability profiles of two mRNAs (C. elegans-Category I, closed squares and D. melanogaster, closed circles) and the profiles of three collections of random 19-nucleotide duplex RNA molecules. Random collections of 19-nucleotide duplex RNAs were obtained by hybridizing segments of 19-nucleotide antisense oligonucleotides to mRNA sequences of two human genes [Protein kinase C-theta (PKC-θ, open squares) and Insulin-like growth factor receptor 1 (IGF1-R, Open diamonds] and a reporter gene, secreted alkaline phosphatase (SEAP, open circles). Sample sizes of the three collections of random 19-nucleotide duplexes were 189 for IGF1R, 100 for PKC-θ and 70 for SEAP. (C) Calculated average Tₘ values for the four categories of mRNAs. Average Tₘ was obtained by averaging the calculated Tₘ values of individual mRNA duplexes in each category. Error bars indicate the average deviation.

FIG. 17 shows the calculated average internal stability profiles and calculated Tₘ values for certain experimentally validated SiRNA molecules targeted to six human genes, one mouse gene, and a single reporter gene, SEAP. (A) Calculated average internal stability profiles of functional (closed squares; sample size of 16) and nonfunctional (open squares; sample size of 21) SiRNA molecules. (B) Calculated Tₘ values of individual SiRNA molecules used for experimental validation of target mRNA reduction. Filled bars indicate Tₘ values of functional SiRNA molecules, whereas the open bars show those of nonfunctional SiRNAs. For each SiRNA molecule, the first part of the name indicates the targeted gene and the number reflects the first nucleotide of the target site within the mRNA sequence. (C) Calculated average internal stability profiles of functional (closed squares; sample size of 12) and nonfunctional (open squares; sample size of 8) SiRNA molecules with calculated Tₘ values between 50°-70° C.

FIG. 18 shows the calculated individual internal stability profiles for representative examples of functional (closed squares) and nonfunctional (open squares) SiRNA molecules that do not satisfy both criteria for a functional SiRNA. (A) SiRNA (PKB-α-1436; open squares) has high internal stability and a high value for the calculated Tₘ (85° C.), and is nonfunctional. SiRNA (PKC-θ-1303; closed squares) is a functional trigger that barely satisfies the internal stability profile, but has a low value for the calculated Tₘ (66° C.). (B) Neither of the two SiRNAs has a preferred internal stability profile. SiRNA (TERT-708; open squares) has a relatively high value for the calculated Tₘ (79° C.) and is non functional. SiRNA (DJ-1-615; closed circles) has a low value for the calculated Tₘ and is functional. (C) Internal stability profiles of three SiRNA molecules targeted to overlapping sites (SEQ ID NOS.22 to 24 and 151) (illustrated at the top of the graph) within the mRNA of PKC-θ. SiRNA-1 (PKC-θ-69) and SiRNA-2 (PKC-θ-70) are both functional (filled squares) and have a preferred bell-shaped internal stability profile. In contrast, SiRNA-2 (PKC-θ-75) that targets only five nucleotides downstream has an undesirable internal stability profile (open circles) and is nonfunctional. This is in spite of its calculated Tₘ value of 55° C., which is well within the Tₘ range of the other two functional SiRNA molecules.

FIG. 19 shows the characteristics of certain SiRNA triggers (rationally designed and arbitrarily picked) for SEAP mRNA. Panel A shows internal stability profiles and sequences of rationally designed SiRNA triggers based on criteria outlined here. A1 shows SiRNA triggers (SEQ ID NOS. 25 to 32) that were designed to be functional, whereas A2 indicates those (SEQ ID NOS. 33 to 36) designed not to be functional. Panel B shows internal stability profiles and sequences of arbitrarily (randomly) picked SiRNA triggers. B1 shows the SiRNA triggers (SEQ ID NOS. 0.37 to 40) picked arbitrarily that turned out to be functional (SP-155, which reduces the SEAP level by approximately 50%, is considered marginally functional), whereas B2 illustrates all the nonfunctional SiRNA molecules (SEQ ID NOS. 41 to 48). For each SiRNA, a calculated internal stability profile of the antisense strand (top) and the Tₘ of the duplex is shown. Panel C shows the endpoint measurements of RNA interference elicited by different SiRNA triggers that were either rationally designed or arbitrarily picked. The enzyme activity of cell medium after transient expression of the SEAP gene using prAAV6-SEAP plasmid in the presence of different SiRNA molecules (at 100 nM) was measured using a chemiluminescent substrate. The black bars (left) indicate the SEAP activity with rationally designed SiRNA molecules. Bars depicted on the right side of the figure labeled “Random Picks” indicate the enzyme activity with arbitrarily designed SiRNA molecules. The open bar shows the SEAP activity of the control transfection in which no SiRNA was used. Each result is an average of three independent transfections, and the error bar indicates the standard deviation of the three readings.

FIG. 20 shows the concentration dependence of certain SiRNA triggers in silencing SEAP expression. Transfections were carried out as described in FIG. 19, but a range of concentrations for each SiRNA trigger were used. Twenty-four hours after transfection, alkaline phosphatase activity was quantified using a chemiluminescent assay. (A) For each trigger, the SEAP activity is plotted against the concentration of the SiRNA trigger used. Black and gray bars show the SEAP activity in the presence of varying concentrations of SEAP-1035 and SEAP-1070, respectively. The white bars indicate the effect of different concentrations of the most effective SiRNA trigger, SEAP-2217. The two bars to the right show the control in which no SiRNA trigger was included during transfection. (B) A different representation of the data in Panel A. Squares and triangles indicate the concentration effect of SEAP-1070 and SEAP-1035, respectively. Circles show the concentration effect of SEAP-2217.

FIG. 21 shows screen snapshots depicting windows of the Oligo 5.0™ Primer Analysis Software program used for identifying effective SiRNA sequences. The “current oligo length” is set to 19. (A) The melting temperature window shows the calculated Tₘ profiles for 19 nucleotide long duplexes annealed to the mRNA sequence (SEQ ID NOS. 49 and 50). (B) The internal stability window shows the internal stability profile with a target site having a desired bell-shaped curve indicated in open circles for a “good pick”, (C) A second representative internal stability window highlights, in open circles, the undesirable internal stability profile reflecting a poor choice for a SiRNA pick.

FIG. 22 shows the effect of the length of the helical region in certain SiRNA triggers. Sequences of SiRNA triggers with varying length of the RNA helical region (17-, 19-, 21-, 23-, 25-base pair RNA helical region) are shown on the right (SEQ ID NOS. 31, 32, and 51 to 60). Each trigger, except for SP-19-AR, has two deoxy thymidine residues at the 3′ end on each strand (shown in bold). SP-19 is the same as 2217. In SP-19-AR the two nucleotides at the 3′ end in the antisense strand are complimentary to the targeted SEAP mRNA. The effect of these triggers in silencing the SEAP expression is shown on the left. Each measurement is an average of triplicate readings derived from independent transfections of SiRNA triggers at a 100 nM concentration. The control bar reflects the amount of SEAP activity measured in cells in the absence of any SiRNA trigger.

FIG. 23 shows the silencing of SEAP expression by certain SiRNA triggers with different end structures. Sequences of SiRNA triggers used in the experiment are shown to the right (SEQ ID NOS. 31, 32, 59 to 66). Deoxy-nucleotides at the 3′ ends of SP-19 and SP-19-Blunt are underlined and marked in bold. The effect of the silencing of the SEAP gene by these triggers is shown to the left. Each measurement is an average of triplicate readings derived from independent transfections of SiRNA triggers at a 100 nM concentration. The control bar reflects the amount of SEAP activity measured in cells in the absence of any SiRNA trigger.

FIG. 24 shows the primary and secondary structures of certain hairpin triggers carrying four and eight nucleotides in the loops (SEQ ID NOS. 67 to 70). Antisense and sense strands have reverse orientations in SP-HP uucg AS-S and SP-HP uucg S-AS hairpin triggers carrying tetra loops. SP-HP loop 8 AS-S is identical to SP-HP uucg AS-S except that it has an eight-nucleotide loop. SP-HP uucg AS-S+5′ ext has an internal bulge at the base of the stem. In each trigger, the sequence region within the box depicts the antisense and sense strands annealed to form a 19-base pair helical region. Antisense and sense strands are indicated by AS and S.

FIG. 25 shows the effect of the silencing of the SEAP gene by certain triggers. Each measurement is an average of triplicate readings derived from independent transfections of SiRNA triggers at a 100 nM concentration. The control bar reflects the amount of SEAP activity measured in cells in the absence of any SiRNA trigger. 2217 shows the reduction of SEAP level with a double-stranded SiRNA trigger carrying a 19-base pair helical region with two dT residues at the 3′ extensions as another control.

FIG. 26 (A, B, and C) shows eight series of SiRNA triggers in which the variation of antisense strand was studied by keeping the sense strand constant in each series. In each series, SiRNA triggers that are nonfunctional are indicated within a box. In FIG. 26A, the left column contains SEQ ID NOS. 31, 51, 52, 58, 60, 62, 71, 72, and 73. In FIG. 26A, the middle column contains SEQ ID NOS. 31, 32, 52, 58, 60, 62, and 71 to 73. In FIG. 26A, the right column contains SEQ ID NOS. 31, 52, 58, 60, and 71 to 74. In FIG. 26B, the left column contains SEQ ID NOS. 31, 52, 58, 60, 62, 71 to 73, and 75. In FIG. 26B, the right column contains SEQ ID NOS. 31, 52, 57, 58, 60, and 72 to 73. In FIG. 26C, the left column contains SEQ ID NOS. 31, 52, 58 to. 60, 62, and 71 to 73. In FIG. 26C, the middle column contains SEQ ID NOS. 31, 52, 58, 60, 62, 71 to 73, and 76. In FIG. 26C, the right column contains SEQ ID NOS. 31, 52, 58, 60, 62, 71 to 73, and 77.

FIG. 27 shows the effect of the silencing of the SEAP gene by certain triggers whose structures are shown in FIGS. 26A-C. Results are provided for different double stranded RNA molecules with varying lengths and end structures. The sense strands of each molecule are identified in the box below the bar graph. Each sense strand is matched with a series of antisense strands identified in the box to the right of the bar graph as indicated by the shading of the bars. Each measurement is an average of triplicate readings derived from independent transfections of SiRNA triggers at a 100 nM concentration.

FIG. 28 (A, B, and C) shows eight series of SiRNA triggers in which the variation of sense strand was studied by keeping the antisense strand constant in each series. In each series, SiRNA triggers that are nonfunctional are indicated within a box. In FIG. 28A, the left column contains SEQ ID NOS. 32, 51, 52, 57, 59, and 74 to 77. In FIG. 28A, the middle column contains SEQ ID NOS. 31, 32, 51, 57, 59, and 74 to 77. In FIG. 28A, the right column contains SEQ ID NOS. 32, 51, 57, 59, 71, and 74 to 77. In FIG. 28B, the left column contains SEQ ID NOS. 32, 51, 57, 59, 72, and 74 to 77. In FIG. 28B, middle column contains SEQ ID NOS. 32, 51, 57 to 59, and 74 to 77. In FIG. 28B, the right column contains SEQ ID NOS. 32, 51, 57, 59, 60, and 74 to 77. In FIG. 28C, the left column contains SEQ ID NOS. 32, 51, 57, 59, and 73 to 77. In FIG. 28C, the right column contains SEQ ID NOS. 32, 51, 57, 59.62, and 74 to 77.

FIG. 29 shows the effect of the silencing of the SEAP gene by triggers whose structures are shown in FIGS. 28A-C. Results are provided for different double stranded RNA molecules with varying lengths and end structures. The antisense strands of each molecule are identified in the box below the bar graph. Each antisense strand is matched with a series of sense strands identified in the box to the right of the bar graph as indicated by the shading of the bars. Each measurement is an average of triplicate readings derived from independent transfections of SiRNA triggers at a 100 nM concentration.

FIG. 30 depicts several dsRNAs having C-U substitutions, a positive control dsRNA (SP-1260-s+SP-1260-as), and a negative control dsRNA (SP-19) and their activity in an RNA interference assay (SEQ ID NOS. 29 to 32, 35, 36, and 78 to 81).

FIG. 31 shows certain antisense oligonucleotides (ASOs) and ds-SiRNA triggers used to target PKC-θ mRNA at three different sites, site #4, #8 and #15, identified by a computer program designed for ASO picking (SEQ ID NOS. 2 to 5, 7 to 15, 82, and 83). In each case, the 25 nucleotide long ASO sequence is underlined. The middle seven nucleotides in the ASOs have a 2′-OMe modification (boxed), whereas the nucleotides that flank the 2′-OMe stretch (nine nucleotides to either side) have a phosphorothioate backbone. Below each ASO are two ds-SiRNAs. Each contains two dT residues in its 3′ extension. The ds-SiRNA designated by A has a 21 nucleotide target homology, whereas the other ds-SiRNA designated by B carries a 19 nucleotide target homology.

FIG. 32 depicts the nucleotide sequences and the predicted fold-back stem-loop structures of certain ss-SiRNA (SEQ ID NOS. 16 to 21) designed to target the same three sites, site #4, #8 and #15, as indicated in FIG. 31. For each site, two ss-SiRNA triggers were designed. The one designated by HP contains six dT residues in the loop and two dT nucleotides in the 3′ extension of the predicted folded stem-loop structure, and was obtained by chemical synthesis. The other, denoted by HP-T, contains six U residues in the loop and a 5′ extension was produced by in vitro transcription. In these molecules, underlined nucleotides do not share homology to the target site.

FIG. 33 shows the quantification of the mRNA level of PKC-θ in 293 cells 24 hours after transfection with certain nucleic acid triggers. The amount of chemiluminescence (RLU) is directly proportional to the amount of PKC-θ mRNA in a single well of a microtiter plate. Each measurement is an average of triplicate measurements derived from independent transfections with error bars representing the standard deviation of the three measurements. Bars: 1, 2, 9, 10, 17, 18, 25, and 26 indicate the PKC-θ levels after transfection with ASO; 3, 4, 11, 12, 19, 20, 27, and 28 indicate the PKC-θ levels after transfection with ds-SiRNA; 5, 13, and 21 indicate the PKC-θ levels after transfection with chemically synthesized ss-SiRNA; 14, 15, 22, and 23 indicate the PKC-θ levels after transfection with in vitro transcribed ss-SiRNA; 6 and 7 indicate the PKC-θ levels after transfection with ss-SiRNA with a single nucleotide deletion; 31 indicates the PKC-θ levels after transfection with the scrambled ASO.

FIG. 34 shows the quantification of the Cyclophilin mRNA level in 293 cells 24 hours after transfection with certain nucleic acid triggers. The amount of chemiluminescence (RLU) is directly proportional to the amount of Cyclophilin mRNA in a single well of a microtiter plate. Each measurement is an average of triplicate measurements derived from independent transfections with an error bar representing the standard deviation of the three readings. Bars: 1, 2, 9, 10, 17, 18, 25, and 26 indicate the Cyclophilin levels after transfection with ASO; 3, 4, 11, 12, 19, 20, 27, and 28 indicate the Cyclophilin levels after transfection with ds-SiRNA; 5, 13, and 21 indicate the Cyclophilin levels after transfection with chemically synthesized ss-SiRNA; 14, 15, 22, and 23 indicate the Cyclophilin levels after transfection with in vitro transcribed ss-SiRNA; 6 and 7 indicate the Cyclophilin levels after transfection with ss-SiRNA with a single nucleotide deletion; 8, 16 and 24 indicate the Cyclophilin levels after transfection with RNA antisense strand alone; 29 and 30 indicate Cyclophilin levels after no transfection; 31 indicates the Cyclophilin levels after transfection with the scrambled ASO.

FIG. 35 shows the selective reduction of PKC-θ message in 293 cells 24 hours after transfection with certain nucleic acid triggers. The level of PKC-θ mRNA (in FIG. 33) was normalized by dividing the level of PKC-θ by the level of Cyclophilin mRNA (in FIG. 34). Bars: 1, 2, 9, 10, 17, 18, 25 and 26 indicate the PKC-θ levels after transfection with ASO; 3, 4, 11, 12, 19, 20, 27 and 28 indicate the PKC-θ levels after transfection with ds-SiRNA; 5, 13, and 21 indicate the PKC-θ levels after transfection with chemically synthesized ss-SiRNA; 14, 15, 22 and 23 indicate the PKC-θ levels after transfection with in vitro transcribed ss-SiRNA; 6 and 7 indicate the PKC-θ levels after transfection with ss-SiRNA; 6 and 7 indicate the PKC-θ levels after transfection with a single nucleotide deletion; 8, 16 and 24 indicate the PKC-θ levels after transfection with RNA antisense strand alone; 29 and 30 indicate the PKC-θ levels after no transfection; 31 indicates the PKC-θ levels after transfection with the scrambled ASO.

FIG. 36 shows the cytotoxity of 293 cells 24 hours after transfection with certain nucleic acid triggers. The cytotoxicity was measured by the MTT assay. Each measurement is an average of triplicate measurements derived from independent transfections with an error bar representing the standard deviation of the three readings. Bars: 1, 2, 9, 10, 17, 18, 25 and 26 show the toxicity levels after the transfection with ASO; 3, 4, 11, 12, 19, 20, 27 and 28 show the toxicity levels after transfection with ds-SiRNA; 5, 13, and 21 show the toxicity levels after transfection with chemically synthesized ss-SiRNA; 14, 15, 22 and 23 show the toxicity levels after transfection with in vitro transcribed ss-SiRNA; 6 and 7 show the toxicity levels after transfection with ss-SiRNA with a single nucleotide deletion; 8, 16 and 24 show the toxicity levels after transfection with RNA antisense strand alone; 29 and 30 show toxicity levels with no transfection; 31 and 32 indicate the toxicity levels after the transfection with the scrambled ASO.

FIG. 37 shows the cytotoxity of 293 cells 24 hours after transfection with certain nucleic acid triggers. The toxicity was measured by the AlamarBlue assay. Each measurement is an average of triplicate measurements derived from independent transfections with an error bar representing the standard deviation of the three readings. Bars: 1, 2, 9, 10, 17, 18, 25 and 26 indicate the toxicity levels after transfection with ASO; 3, 4, 11, 12, 19, 20, 27 and 28 indicate the toxicity levels after transfection with ds-SiRNA; 5, 13, and 21 indicate the toxicity levels after transfection with chemically synthesized ss-SiRNA; 14, 15, 22 and 23 indicate the toxicity levels after transfection with in vitro transcribed; ss-SiRNA; 6 and 7 indicate the toxicity levels after transfection with ss-SiRNA with a single nucleotide deletion; 8, 16 and 24 indicate the toxicity levels after transfection with RNA antisense strand alone; 29 and 30 indicate the toxicity levels after no transfection; 31 and 32 indicate the toxicity levels after transfection with the scrambled ASO.

FIG. 38 shows the reduction of PKC-θ mRNA using certain ds-SiRNA triggers picked by random choice (SEQ ID NOS. 84 to 97). Random pick # 4 is a blunt end ds-RNA sequence. The N19-AA-Rm sequence was picked to have homology throughout the molecule, including the two dT residues at the 3′ end of the antisense strand. This molecule has one blunt end, whereas the other end is likely to be unpaired. Each measurement is an average of triplicate measurements derived from independent transfections with an error bar representing the standard deviation of the three readings. Gray bars indicate the level of mRNA when transfected with ds-SiRNA BSS, a ds-SiRNA trigger that should not have homology to any human gene. Black bars indicate the level of mRNA in cells that were not transfected.

FIG. 39 shows the reduction of the DJ-1 mRNA level in MDA-MB453 cells upon transfection with certain ds-SiRNAs and ASOs (SEQ ID NOS. 96 to 107). Each measurement is an average of triplicate measurements derived from independent transfections with an error bar representing the standard deviation of the three readings. Grey bars indicate the level of mRNA when transfected with ds-SiRNA BSS, a ds-SiRNA trigger that should not have homology to any human gene. Black bars indicate the level of mRNA in cells that were not transfected.

FIG. 40 shows the reduction of KD312 mRNA level in DLD-1 cells upon transfection with ds-SiRNAs and ASOs (SEQ ID NOS. 108 to 113). Ds-SiRNAs are based on the two antisense sequences. Each measurement is an average of triplicate measurements derived from independent transfections with an error bar representing the standard deviation of the three readings. The bar at the far right indicates the level of mRNA in cells that were not transfected.

FIG. 41 depicts plasmid pAAV6-seap used in transient transfection studies.

FIG. 42 shows the silencing of the transient expression of SEAP in 293 cells using certain ds-SiRNAs (SEQ ID NOS. 114 to 119). The activity of SEAP produced after the transfection of pSEAP-AAV into 293 cells either in the presence of: 1. ds-SiRNA-Seap-1 (open), 2. ds-SiRNA-Seap-2 (gray), 3. ds-SiRNA-Seap-3 (black) or absence of any SiRNA trigger (indicated as “None” on the far right of the graph) was quantified using a chemiluminescent substrate. Each data point is an average of three independent transfections with an error bar representing the standard deviation of the three readings.

FIGS. 43A, B and C show the duration of certain RNA interference in the transient expression of a SEAP reporter gene in 293 cells. SEAP activities are shown after co-transfection of different amounts of pMV6-seap plasmid (squares-25 pmoles, circles-5 pmoles, diamonds-0.5 pmoles, triangles-0.05 pmoles) with three different concentrations of ds-SiRNA-Seap-1 (Panel A-100 nM, Panel B-10 nM and Panel C-5 nM). After the indicated time, 15 μL of the medium was used to assay for the activity of SEAP. Each data point is an average of three independent transfections.

FIG. 44 shows the duration of certain RNA interference and antisense effects in reducing PKC-θ mRNA level in 293 calls. Cells were transfected with either ASO AS-8 shown in FIG. 31 (filled bars) or ds-SiRNA-8-A, shown in FIG. 31 (open bars). The message level of PKC-θ was quantified after 12 hours. (Panel A), 24 hours. (Panel B) and 72 hours. (Panel C). In each panel, the gray bars indicate the control experiment in which nonspecific ASO was used.

FIG. 45 shows the certain ds-SiRNAs used for studying the nature and the specificity of SiRNA triggers (SEQ ID NOS. 4, 5, and 120 to 141). These triggers are designed to interact with the PKC-θ message and contain two dT residues at their 3′ ends. Mut-1 and Mut-2 triggers carry one and two base-pair mutations as indicated by bold and underlined nucleotides. Ds-SiRNA triggers 1-3 have 12, 15, and 17 base pairs that are homologous to the target mRNA. Trigger 1 is 14 nucleotides long. Trigger 2 is 17 nucleotides long. Trigger 3 is 19 nucleotides long. Triggers 4-6 have different lengths of target homology, but the physical length of all three triggers is kept at 21 nucleotides. In triggers 4-6, those nucleotides not homologous to the target mRNA are shown in lower case letters. Trigger 7 is the same as ds-SiRNA-BA, shown in FIG. 31. Triggers 8-13 have point mutations in the sense strand while the antisense strand is completely homologous except for two dTs at the 3′ end. In each case the point mutation(s) in the sense strand is underlined and indicated by bold.

FIG. 46 depicts the reduction of the PKC-θ message level by certain ds-SiRNA triggers with different characteristics. Sequences of these triggers are illustrated in FIG. 45. The effect of the physical length of the SiRNA trigger is shown by A (1-12 base-pairs, 2-15 base-pairs, and 3-17 base pairs). B indicates the length of the homology requirement of ds-SiRNA triggers. Triggers 4, 5 and 6 carry 21-base pairs, out of which the length of target homology varies from 17 (4), 15 (5) and 12 (6). The effect of triggers carrying single (Mut-1) and double (Mut-2) base pair changes on the reduction of the PKC-θ message is indicated by X and Y, respectively. C indicates the effect of mutations located only in the sense strand. Ds-SiRNA trigger 7 is the positive control in which there is no mutation in the sense strand, whereas triggers 8-13 carry 1, 2, 3, 4, 5 and 6 point mutations in the sense strand, respectively. The black bar indicated by 14 shows the PKC-θ message level in the control experiment where no SiRNA was used.

FIG. 47 shows the reduction of the PKC-θ message level by a ds-SiRNA (ds-SiRNA-8-A) trigger carrying different chemical modifications. As indicated, ds-SiRNA was modified at the 2′-position in the sugar with Fluorine (F), Methoxy (OMe) and H (DNA). In the case of 2′-F modification, only the pyrimidines were modified, whereas the modifications were introduced throughout the strand with the other two modifications. All three combinations, modifications in the antisense sense strand (1), sense strand (2), and both strands (3) were used. Combinations represent a 2′-F modification in the antisense and H- and OMe-modifications in the sense strand. In addition to the modification in the sugar, the 3′ ends were also modified with two different caps; inverted dT and inverted abasic modifications.

FIG. 48 depicts certain unimolecular (ss-SiRNAs) and bimolecular (ds-SiRNAs) triggers designed for the SEAP mRNA. All pyrimidines in Sphp-1 F are modified with a 2′-F group (SEQ ID NOS. 142 to 150).

FIG. 49 shows the silencing of the transient expression of SEAP in 293 cells using certain bimolecular and unimolecular SiRNAs. The activity of SEAP produced after the transfection of pSEAP-AAV into 293 cells in the presence of different RNAi triggers was quantified using a chemiluminescent substrate. Each data point is an average of three independent transfections with an error bar representing the standard deviation of the three readings.

FIG. 50 shows genes targeted by certain arbitrarily designed SiRNA molecules. The column on the far right labeled “% Success” shows the number of arbitrarily picked SiRNAs that successfully mediated RNA interference of the target gene as a percentage of the total number of SiRNAs that were arbitrarily picked for that target gene. The G/C content is calculated as the G/C content of the SiRNA.

### DETAILED DESCRIPTION OF CERTAIN EMBODIMENTS OF THE INVENTION

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Standard techniques may be used for recombinant DNA, oligonucleotide synthesis, and tissue culture and transformation (e.g., electroporation, lipofection, etc.). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

The following examples, including the experiments conducted and results achieved are provided for illustrative purposes only and are not to be construed as limiting the present invention in any way.

I. Gene Inactivation Through RNA Interference (RNAi)

Introduction

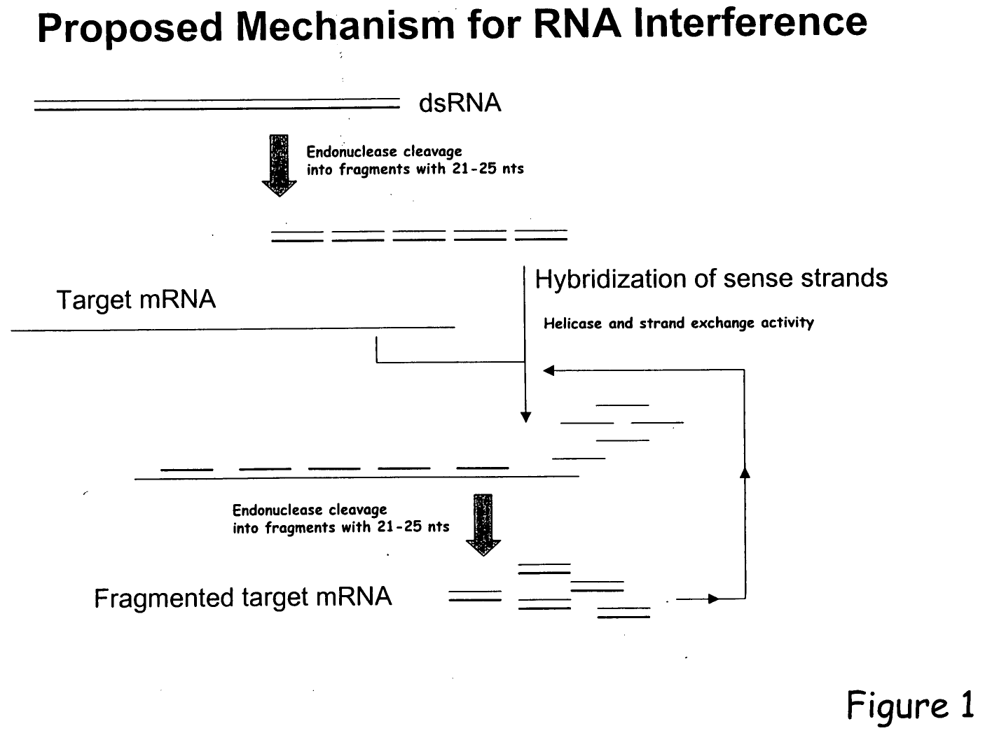

Double-stranded RNA (dsRNA)-mediated specific gene inactivation or gene silencing has been observed in many different organisms. This phenomenon, referred to as RNA interference (or RNAi), is expected to play a significant role in understanding gene function, signal transduction pathways and identifying therapeutic agents in the future. To date, the mechanism by which a dsRNA molecule inactivates the expression of a gene is not completely clear. Certain experimental evidence indicates that the process of RNA interference may be post-transcriptional and involves degradation of specific mRNA. The mRNA that is degraded is dictated by the sequence of the dsRNA that is introduced into the cell. As a result, the RNA interference process has been shown to be extremely sequence-specific. It has been observed that the dsRNA that mediates the target mRNA degradation is processed (or cleaved) into a collection of fragments of 21-25 nucleotides. According to a proposed model (Zamore, P. D. et al., Cell, 101, 25-33, 2000; Bass, B. L., Cell, 101, 235-238, 2000), the sense strands of RNA fragments derived from the dsRNA are hybridized to the target mRNA. This process is believed to be facilitated by a helicase and a protein catalyzing an ATP-dependent strand exchange activity. The target mRNA is subsequently cleaved, resulting in the inactivation of the message before being translated (FIG. 1).

In certain embodiments RNA interference may be used in the following areas.

1) To use as a tool for target validation in functional genomics.

2) To generate therapeutic molecules that interfere in specific gene expression.

3) To generate transgenic animals and plants by introducing gene constructs that express dsRNA targeted to a given gene.

4) To use as a technique for gene therapy applications in controlling the expression of targeted genes.

5) To use in oligonucleotide therapeutics using synthetic nucleic acid molecules.

1. For Functional Genomics and Therapeutics

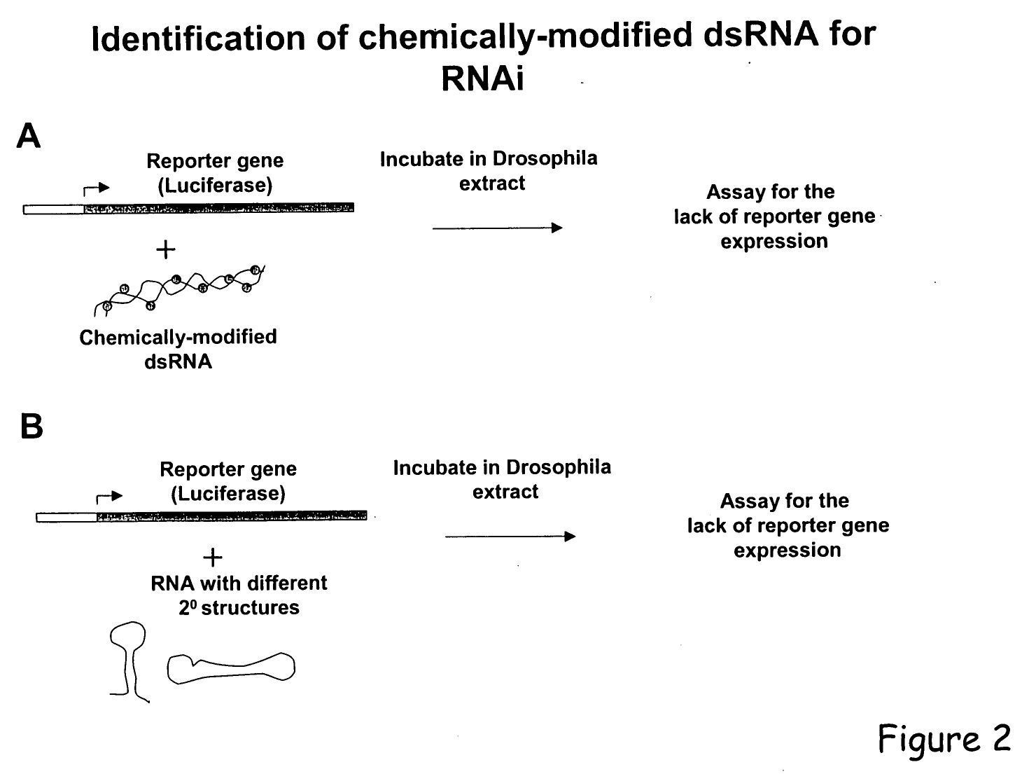

An RNAi technique is envisioned to play a role in functional genomics to validate gene targets in both tissue culture and animal models. At the tissue culture level, inactivation of a target gene has been demonstrated by introducing into cells exogenous dsRNA that has been produced by in vitro transcription. These natural dsRNAs are relatively long molecules consisting of 100-800 base pairs. It may be beneficial to identify RNA species that are more nuclease resistant than natural RNA. In certain embodiments, one may screen a variety of chemically-modified RNAs to identify candidates that are both nuclease resistant and active in the RNAi mechanism (FIG. 2). In certain instances, one may identify chemically-modified, thereby nuclease resistant, RNA species that are both active in RNA interference and amenable for economical chemical synthesis. Certain relatively small chemically-modified RNA species with appropriate secondary structures that will make them active in RNA interference are discussed. One may find suitable modifications in RNA in light of the recapitulation of the essential features of RNAi in vitro using a cell-free system derived from Drosophila embryos (Tuschl, T. et al., Genes and Development, 13, 3191-3197,1999). The cell-free system would enable identification of chemically modified RNA species that retain the RNAi activity. Furthermore, the same system could be used to design a dsRNA molecule that is short, economical and effective in RNA interference. For example, these RNA molecules may have short stem-loop structures.

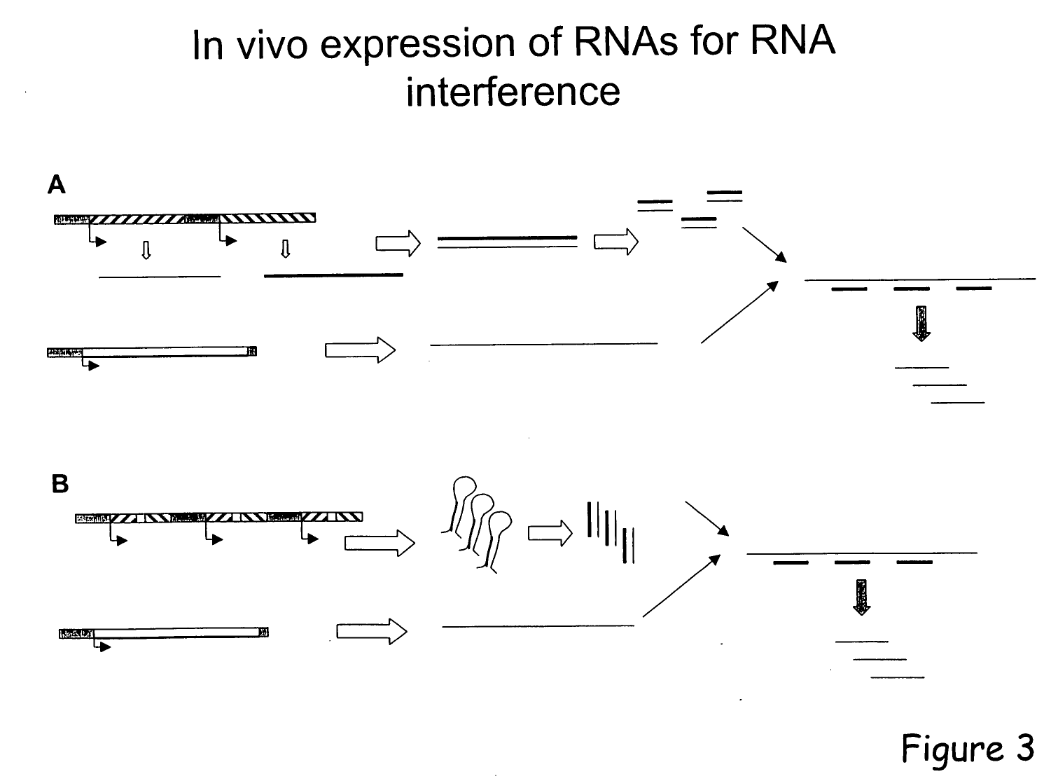

In certain instances, the dsRNA molecules that mediate the degradation of a specific mRNA may be expressed in vivo (FIG. 3). They may be expressed as fairly long RNA transcripts that could form stem-loop structures. One strand that forms the stem will have the sequence complementary (antisense) to the target mRNA. Alternatively, sense and antisense strands of the dsRNA may be expressed separately under two promoters. In these applications, it is envisioned to use transcriptionally active but translationally inactive cassettes to express RNA in vivo. The present disclosure describes the use of RNA expression cassettes in vivo. These will include, but are not limited to, the use of promoters for RNA polymerase II and III as well as any viral promoters.

In certain embodiments, a host cell may be contacted with two or more interfering RNAs that may target one or more target mRNAs.

Although an antisense approach may be used in target validation using tissue culture models, the technology sometimes may be challenging in validating the same targets in animal models. This may be due to poor delivery and targeting of short synthetic oligonucleotides to desired tissues in animals. Expression of dsRNA molecules in animals using gene delivery vectors (Examples: Retroviral, Adenovirus and Adeno Virus-Associated vectors) may obviate this challenge and is a subject of this disclosure. Hence, an RNA interference approach could represent a method of choice for validating target genes in animal models.

2. Generating Transgenic Animals and Plants

Transgenic species that do not express certain genes could be produced by specific introduction of DNA cassettes that transcribe dsRNA specific to messages of genes of interest. In certain embodiments, viral resistant transgenic species may be produced by introducing dsRNA specific to certain viruses known to be pathogenic in that species.

3. Gene Therapy Applications

The RNA interference approach could be useful in inhibiting a gene product in patients using a gene therapy approach. For example, genes that are responsible for a multi drug resistance phenotype may be a useful target for an RNA interference approach. This has a direct impact in cancer patients undergoing chemotherapy.

Advantages of an RNAi Approach:

1. Accumulating experimental evidence suggests that the process of RNA interference may be a catalytic process mediated by interplay of several proteins. As a result, only a few copies of dsRNA per cell may be required to degrade a large pool of target RNA.

2. Double-stranded RNA molecules that carry out gene inactivation could be either introduced exogenously or expressed in vivo, facilitating the mode of delivery for different applications.

3. Double-stranded RNA molecules are readily taken up by cells. Unlike certain single-stranded oligonucleotides currently used in antisense research.

Technologies that allow specific inhibition of gene expression are becoming important for drug discovery efforts to identify better human therapeutics. Such techniques may play an important role in the following specific areas.

1. Identifying novel genes with implications in disease states.

2. Identifying specific biological roles of a gene of interest.

3. Characterizing complex biological pathways in details.

4. Establishing experimental animal models to move drug candidates to clinics.

Furthermore, in addition to their role in facilitating drug discovery, molecules that mediate specific gene inhibition could become therapeutics as well. One widely used technique in target validation in cultured mammalian cells has been the antisense approach (reviewed in (Murray, 1992)) in which the specific destruction of target mRNA is triggered by the delivery of a ssDNA molecule that is complimentary to a region within the target. Upon hybridization of the antisense (AS) oligonucleotides to its target mRNA, it is believed that the enzyme RNase H is recruited to the site to cleave the message.

To compare the characteristics of RNA interference with those of antisense-mediated gene knockdown, PKC-θ, an endogenous gene expressed in the human kidney cell line HEK 293 (293 cells), was used in the following experiment as the primary target. After the delivery of either SiRNA triggers or antisense oligonucleotides, the level of PKC-θ message was measured by a branched DNA (bDNA) assay, an assay designed to capture and detect a specific nucleic acid sequence such as a unique mRNA. In parallel, cellular toxicity and nonspecific gene inactivation was studied by monitoring the level of a housekeeping gene, cyclophilin. In addition to these studies, experiments were designed to gain further understanding of the nature of SiRNA triggers with respect to their specificity, homology, length requirements, and their tolerance to a range of chemical modifications.

Materials and Methods

SiRNA

SiRNAs were prepared using several different methods. Certain chemically synthesized SiRNAs were synthesized using RNA phosphoramidites containing a 2′-O-TriisopropylsilylOxyMethyl (TOM) protection group from Glen Research (Sterling, Va.). Other SiRNAs were obtained from Dharmacon (Longmont, Colo.) employing 5′-Silyl-2′-bis(2-acetoethoxy)methyl (ACE) Orthoester chemistry. Synthesized SiRNAs using TOM phosphoramidites were HPLC purified, whereas those obtained from Dharmacon were used without further purification, due to their high purity resulting from extremely high coupling efficiency (Scaringe, 2001). Ss-SiRNAs produced by in vitro transcription were obtained using corresponding synthetic DNA templates containing the promoter for the T7 RNA polymerase (Milligan et al., 1987). After transcription, full-length RNAs were purified by polyacrylamide gel electrophoresis run under denaturing conditions. SiRNAs were annealed in an annealing buffer consisting of 100 mM KCl, 30 mM HEPES (pH 7.5), and 2 mM MgCl₂ by heating to 75° C. for 2 minutes followed by slow cooling to ambient temperature. HPLC-purified antisense oligonucleotides (ASO) were obtained by solid phase synthesis using cyanoethyl phosphoramidites. The ASOs contained two chemical modifications: the middle seven nucleotides carried a 2′-OMe modification in the sugar and the remaining flanking regions contained phosphorothioate linkages.

Plasmid Expressing Secreted Alkaline Phosphatase (SEAP)

The SEAP gene (from pAP-1 SEAP vector from Clontech) was cloned into an adeno-associated vector (AAV # 6).

Cultured Mammalian Cells

This work used the following cell lines cultured in DMEM with 10% FBS at 5% CO₂ at 37° C.

| Cell line | ATCC number |
|---|---|
| HEK 293 | CRL1573 |
| DLD-1 | CCL221 |
| MDA-MB 453 | HTB131 |
| TM3 | CRL1714 |
| Calu 6 | HTB-56 |

Delivery of Nucleic Acid Triggers

Cells seeded in 96-well plates at approximately 25,000/well in the previous day were transfected with different nucleic acid triggers using Lipofectamin 2000 and Opti-MEM I (from Invitrogen). Briefly, SiRNA was diluted in Opti-MEM-I in 50 μL of volume. This was mixed with an equal volume of Lipofectamin 2000 diluted 12.5-fold in OPTI-MEM I. After incubating the mixture at ambient temperature for 20 minutes, 270 μL of the regular cell medium was added and 95 μL of the solution was immediately transferred onto the cells in the plate with no media. Plates were transferred to a 37° C. incubator with 5% CO₂ for either 24 or 48 hours. To monitor the fate of SEAP transiently expressed in 293 cells, prAAV6-seap plasmid was included in the transfection mixture with and without SiRNAs.

Quantification of mRNA Levels

Specific mRNA levels of cells transfected with different nucleic acid triggers were quantified using QuantiGene High Volume Kit (from Bayer) that employs a branched-DNA (b-DNA) method for nucleic acid detection according to the manufacturer's instructions. Specific detection of a given mRNA is based on its selective capture on to the microtiter plate, which is dictated by the capture probes. Probe sets that are unique to each target mRNA were designed using the ProbeDSesigner software (Bayer) according to the manufacturer's instructions. Before using the probe sets in experiments, the probes were tested using the cells expressing each message to make sure that they worked.

Cytotoxicity Assays

After 24 or 48 hour incubation, 25 μL of AlamaBlue reagent (Trek Diagnostic Systems, Inc.) was added to each well and incubated at 37° C. with 5% CO₂ for 2 hours. The absorbance at 570 nm was read in a SpectraMax UVNIS 96-well plate forrriat spectrophotometer. The MTT assay was carried out according to the manufacturer's instructions.

Secreted Alkaline Phosphatase (SEAP) Assay

Twenty-four hours after the transfection, 15 μL of medium from each well was transferred to a white opaque 96-well flat bottom microtiter plate, and the amount of SEAP was detected using chemiluminescent SEAP assay (Great EscAPe SEAP assay kit form Clontech) according to the manufacturer's instructions.

Results and Discussion

Design of SiRNA Triggers

SiRNA Triggers Based on Antisense Target Sites

The initial set of SiRNA triggers were based on three antisense DNA sequences that target three independent sites in the PKC-θ message. A computer program designed to identify the best target sites for antisense oligonucleotides had previously picked these sites. When targeted by antisense DNA molecules, sites 8 and 15 were more effective than site 4 in reducing the level of PKC-θ message. FIG. 31 shows the sequences of antisense DNA sequences as well as ds-SiRNA triggers used in the study. All antisense DNA sequences used are 25 nucleotides in length and contain 2′-OMe substitutions in seven nucleotides in the middle. This region is flanked by nine phosphorothioate nucleotides to either side. Similar to the design reported by Elbashir, 2001 a, the Ds-SiRNA molecules also contained 3′-overhangs having two deoxythymidine (dT) residues. In the previous design by Elbashir, 2001 a, the two dT residues in the 3′-overhang of the antisense strand provided enhanced nuclease stability while maintaining target recognition. (Elbashir, 2001a) In contrast, the dT residues in the 3′-overhang of the antisense strand of the ds-SiRNA triggers here do not base pair with the target mRNA, hence the target homology is shortened by two nucleotides. For each target site, two sets of ds-SiRNA triggers with 21 and 19 nucleotide (nts) homology to the target sequence were designed.

Silencing of specific genes has been observed upon the introduction of RNA with the capacity to form long hairpin molecules in which one half of the folded sequence is homologous to the mRNA of the targeted gene (Piccin, 2001; Tavernarakis, 2000; Wang et al., 2000). The experiment here sought to determine whether, short chemically synthesized single-stranded RNA (ssRNA) with the propensity to fold-back and form short hairpin molecules may be used as triggers to induce RNAi in vivo. To investigate whether ss-RNA that forms short hairpin structures could trigger RNAi in mammalian cells, two such molecules for each target site were designed (FIG. 32). This class of molecules was named Single-Stranded Short interfering RNA Hairpin (ss-SiRNA-HP) triggers to distinguish them from the bimolecular ds-SiRNA triggers having two separate strands complimentary to each other. Out of the two unimolecular ss-SiRNA-HP triggers designed for each site, one trigger contained six dT residues in the loop and two dT residues in the 3′ overhang and was produced by chemical synthesis. The other trigger containing six uridines in the loop and two uridines at the 3′ recessed end was obtained by in vitro transcription.

Impact of Different Nucleic Acid Triggers Upon Transfection into 293 Cells

We carried out parallel analysis of several end points within the cells transfected by a series of nucleic acids triggers such as single-stranded DNA and RNA antisense molecules, ds-SiRNA, and ss-SiRNA-HP. After delivery of each nucleic acid trigger into 293 cells, the following were measured: (1) reduction of specific mRNA, (2) reduction of nonspecific mRNA and (3) cellular toxicity.

Fate of Target-Specific and Nonspecific mRNA Levels in 293 Cells

The reduction of the specific message PKC-θ was evaluated by measuring the level of PKC-θ mRNA by a bDNA capture-detection method (FIG. 33). This work also measured the message level of the cyclophilin gene, a housekeeping gene, as a control to see any nonspecific effects mediated by different nucleic acid triggers (FIG. 34). The level of target mRNA was normalized to the level of the nonspecific mRNA to show the selective reduction of the target mRNA by each nucleic acid trigger (FIG. 35). As indicated in FIGS. 33 and 35, all three antisense sequences reduced the PKC-θ message level in a dose dependent manner (bars, 1, 2, 9, 10, 17 and 18). A combination of all three antisense DNA molecules did not show synergistic effect at 100 nM total DNA concentration (bars 25 and 26). Ds-SiRNA triggers targeted to all three sites were also effective in reducing the message level (bars, 3, 4, 11, 12, 19 and 20). Very similar to antisense-mediated gene knockdown, the efficiency of gene knockdown by SiRNA triggers is also target site dependent, suggesting that the target accessibility presumably plays a role in the RNA interference process. A combination of all three ds-SiRNA triggers did not exert synergy in reducing the mRNA level (bars 27 and 28). Ss-SiRNA-HP triggers also reduced the level of PKC-θ message (bars, 5, 13-15, and 21-23), indicating that ss-SiRNA-HP triggers with the propensity to fold into stem-loop structures serve as triggers for RNA interference in mammalian cells. Both types of ss-SiRNA-HP triggers, ones derived from chemical synthesis that contain dT residues in the loop, and the others, derived from transcription that contain uridines in these positions, were effective in reducing specific mRNA levels. This indicates that the nature of nucleotides occupying the loop in this work did not affect the RNA interference process. Interestingly, the ss-SiRNA-HP trigger containing a single nucleotide deletion was inefficient in knocking down the level of PKC-0 mRNA (bars, 6 and 7). This result shows here that a specific RNAi trigger initiated an efficient interference process. It is likely that a single point deletion in a stem-loop sequence could lead to the adoption of several alternate conformers with partial base pairing, effectively decreasing the structure illustrated in FIG. 32 for ss-SiRNA-8-HP-T.

A nonspecific effect of nucleic acid triggers on 293 cells was probed by measuring the message level of a housekeeping gene, cyclophilin (FIG. 34). A nonspecific gene knockdown by single-stranded antisense nucleic acid triggers (FIG. 34; bars, 1, 8, 9, 10, 16, 17, 24, and 25) was observed. None of the ds-RNAi molecules targeted at three different sites within the PKC-θ message showed significant effect on the cyclophilin message level, indicating the specificity of RNA interference. On the other hand, antisense DNA directed to all three sites in the PKC-θ message significantly decreased the level of the cyclophilin mRNA. Similar to the single-stranded antisense DNA molecules, single-stranded antisense RNA molecules also showed nonspecific reduction of the housekeeping message level. However, in contrast to the single-stranded antisense RNA, none of the ss-SiRNA-HP triggers showed any effect on the housekeeping message level. Although this current observation is limited to a single gene provided as a nonspecific control, these results suggest a nonspecific effect. Namely, single-stranded antisense molecules, whether they were DNA or RNA, have some degree of nonspecific gene inactivation compared to highly selective gene expression interference mediated by dsRNA. The reason for the specific gene inhibition mediated by RNAi may lie within the heart of its mechanism (Hammond. 2001). On the other hand, the antisense effect is expected to occur by simple complementary strand hybridization without facilitation from a protein, and hence can be prone to nonspecific effects. Alternatively, nonspecific mRNA reduction could be due to the toxicity induced by single stranded nucleic acid triggers upon transfection (see below), sending cells into a state of shock.

No nonspecific gene knock down by ss-SiRNA-HP triggers that could fold back to generate short hairpin structures was observed. This result along with the reduction of the specific message by ss-SiRNA suggests that ss-SiRNA-HP molecules, like ds-SiRNA molecules, function through the RNA interference mechanism to specifically reduce target mRNA levels.

Cellular Toxicity

This work included two commonly used cytotoxicity assays to evaluate cellular toxicity upon delivery of various nucleic acid triggers into cells. Twenty-four hours after the transfection, an AlamarBlue assay was carried out and completed within an hour (FIG. 36). The MTT assay that requires a longer incubation time (overnight) was also initiated after 24 hours of transfection (FIG. 37),

The AlamarBlue assay revealed the toxicity associated with cells transfected with single-stranded antisense molecules, both DNA and RNA (FIG. 36; bars, 1,2,8-10, 16, 17, 24, 25 and 32). The observed toxicity of ss-DNA antisense molecules was dose dependent; more pronounced at 100 nM than 10 nM. An unrelated sequence used as a control also exerted cellular toxicity in the AlamarBlue assay (bar, 32), indicating that the observed toxicity is not a function of antisense sequences directed to PKC-θ mRNA, but rather a general phenomenon. The level of toxicity was also dependent on the number of cells used for the transfection. In general, when greater than 40,000 cells per well were used, the level of toxicity observed with single stranded nucleic acids was significantly reduced (data not shown). This result suggests that the undesirable effect of cellular toxicity presumably caused by the lipid-AS nucleic acid complex gets diluted with increased cell number.

Randomly Picked ds-SiRNA

The above ds-SiRNA triggers were based on target sites picked by a computer program designed to identify the best sites for antisense targeting. They all worked as expected in reducing the target mRNA level by triggering the RNAi mechanism. This work also tested ds-SiRNA targeted to sites picked by random choice, without applying any pre-selected criteria. Several different ds-SiRNA triggers. Three of these had overlapping target sites within the PKC-θ mRNA (FIG. 38A; Random picks # 1-3). Two ds-SiRNA molecules out of these three sharing overlapping target sites were effective in triggering RNAi (FIG. 38b). This result demonstrates that effective ds-SiRNA triggers can be picked randomly. However, not all sites were equally effective in eliciting RNA interference. In this experiment, moving the target site by only five nucleotides made the ds-SiRNA trigger ineffective, suggesting the existence of sites that facilitate RNA interference as well as sites that are inert to the mechanism. Sites that are immune to RNAi could be difficult to access by the RNA induced silencing complex (RISC) due to either the folding of RNA, or the masking of RNA with RNA binding proteins, or some combination of these possibilities.

The two dT residues at the 3′-end of the ds-SiRNAs studied so far do not recognize the target mRNA. These residues were changed to two dC residues and this trigger, containing dC residues in place of dT residues, worked effectively (FIG. 38B). This suggests that the nature of nucleotides in the 3′ extension does not interfere with the process. The ds-SiRNAs are typically synthesized to contain 3′-extensions, mainly to mimic the digested products of exonuclease III Dicer enzyme. However, it is possible that 3′ extensions within ds-SiRNAs are not required to become the substrate for RISC formation and to initiate the RNAi process. This work showed that the 3′-extensions were not necessary to mount an effective RNAi, since triggers with blunt ends worked (FIG. 38; Ds-SiRNA random pick #4). In the case of N19-AA-Rm, one end is blunt and the other is not. These results suggest that the end structure was not important to initiate an effective RNAi process.

By targeting endogenous genes present in several other cell lines (FIGS. 39-40), this work showed that RNAi can work in several mammalian cells. In the case of silencing the DJ-1 gene in MDA MS453 cells (FIG. 39), the ds-SiRNAs based on the optimal antisense sequences were not as effective as those identified by random choice, suggesting that the optimal target site for antisense-mediated gene knockdown does not necessarily become the optimal site for SiRNA.

RNAi in a Transiently Expressed Reporter Gene

The above results were obtained with ds-SiRNA triggers directed against endogenous genes in cultured mammalian cells. This study was extended to a gene expressed transiently upon transfection into mammalian cells. A plasmid was designed to express a reporter gene. SEcreted Alkaline Phosphatase (SEAP), under the CMV promoter (prAAV6-seap) was transfected into 293 cells in the presence and absence of a ds-SiRNA trigger designed to target the SEAP mRNA. In this experiment, three different ds-SiRNA triggers picked randomly to target non-overlapping sites within the mRNA of SEAP were used. Out of the three ds-SiRNA tested, one trigger effectively silenced the transient expression of SEAP (FIG. 41). The RNAi effect induced by ds-SiRNA trigger 1 was very potent and effective at 10 nM. The previous experiments demonstrated the reduction of the mRNA levels of the specific gene that was targeted by SiRNA. The reduction of the target gene, SEAP, correlated with the presence of a ds-SiRNA trigger that targeted the SEAP gene.

Duration of RNA Interference

Next, the duration of RNA interference triggered by a ds-SiRNA in 293 cells was examined. FIG. 43 indicates the period in which RNA interference was effective in silencing a transiently expressed SEAP reporter gene as a function of amounts of prAAV6-seap plasmid and dsSiRNA used. When the amount of plasmid used was low, the persistence of RNAi even up to 170 hours (7 days) after transfection with 100 nM ds-SiRNA was observed (FIG. 43A). With certain increased plasmid concentration, however, the level of expression crept up after 100 hours, and remained at 50% at 170 hours. A similar trend was observed with ds-SiRNA concentrations as low as 5 nM (FIG. 43B &C). Thus, when the target mRNA concentration in cells was relatively low, gene silencing mediated by RNAi persisted longer than with certain higher target mRNA concentrations.

Next, the duration of RNAi in silencing an endogenous gene, PKC-θ (FIG. 44) was investigated. Ds-SiRNA (Ds-SiRNA-8113) was used in the study along with an antisense molecule (AS-8) to measure the duration of RNAi and compare it to that of the antisense effect. After the delivery of nucleic acid triggers, mRNA levels of PKC-θ were measured at 24 hours, 48 hours, and 72 hours, and normalized to the message level of the housekeeping gene at identical time points (FIG. 44A, B and C). Out of the three time points, the strongest effect was observed at 48 hours after transfection. Furthermore, the effect persisted even at 72 hours. The antisense effect, on the other hand, gradually decreased with time. An RNA interference effect peaking at 48 hours may not be a general phenomenon, but could be gene and cell-type specific. Previous studies in mouse zygotes suggested that RNA interference triggered by relatively long ds-RNA (714 bp) lasted for 6.5 days (Wianny, 2000). The efficacy of RNA interference triggered by Ds-SiRNA-813 alone may be generally higher than that induced by Ds-SiRNA-BB mixed with a nonspecific control (data not shown). This result is consistent with previous studies (Parrish, 2000; Tuschl et al., 1999). This suggests that the presence of nonspecific ds-RNA could lead to the formation of unproductive RiSC complexes upon binding to protein components of RiSC. In essence, the addition of nonspecific dsRNA could compete for the limiting amounts of protein components in the cell, thereby decreasing the concentration of the productive RiSC complexes formed with the specific trigger.

Certain SiRNA Triggers

Certain ds-SiRNA triggers may have 21 nucleotides in each strand, out of which 19 nucleotides form base pairs in the duplex leaving two nucleotides in the 3′ extension (Caplen, 2001; Elbashir, 2001 a). In certain designs, all 21 nucleotides in the antisense strand are homologous to the target mRNA. Tests were conducted to test the effectiveness of different lengths of certain ds-SiRNAs, as well as to test the effectiveness of ds-SiRNAs having different homologies. The homology tests involved testing the number of mutations in a ds-SiRNA molecule that may be tolerated certain RNAi processes. These issues were addressed by using a series of ds-SiRNA triggers (FIG. 45).

Homology of the SiRNA Trigger

Certain mutations were introduced in both strands of the ds-SiRNA-8A trigger to obtain two triggers (FIG. 45); Mut-1 has a single base pair change from G-C to A-U, whereas in Mut-2 two G-C base pairs were changed to two A-U base pairs. These mutations were localized in the middle of the trigger. As shown in FIG. 46C, the RNA interference process worked with a single-base pair mutation, although the efficiency may be somewhat reduced. However, mutation of two base pairs was detrimental to the interference process in this work. This result is in agreement with the previous observations made by Elbashir et al., 2001, who observed a lack of RNA interference with ds-SiRNA triggers containing three base pair changes within a 21 nucleotide homologous region. A significant effect caused by a single point deletion in the sense strand of a ss-SiRNA hairpin trigger was observed (see above), indicating that certain ss-SiRNA hairpin triggers could be very sensitive to mutations.

Next, the tolerance of the RNA interference mechanism to the mutations localized only in the sense strand of a SiRNA trigger was investigated. Six ds-SiRNA triggers in which the sense strand carried 1-6 point mutations were constructed (FIGS. 45 and 46, C). Out of these six triggers, only two triggers containing either 1 and 2 point mutations in the sense strand were effective in triggering the RNA interference (FIG. 46, C). This result suggested that the sense strand in this work played an active role in the RNAi mechanism and is not only there to protect the antisense strand by base pairing. It may be that mutations in the sense strand affect the initiation step of the RNAi process, rather than the propagation step. Ds-SiRNA triggers with imperfect base pairing may not be an effective substrate for assembling into the RiSC complex. At least in C. elegans, there is an asymmetry in the two strands with respect to certain chemical modifications (Parrish, 2000); for example the RNAi process accepts substitution of a 2′-NH₂ uracil for regular uracil residues in the sense strand but not in the antisense strand. Thus, in certain instances, these results suggest that both strands of an SiRNA trigger actively participate in the mechanism of the interference process

Short ds-SiRNA Triggers

In certain work, ds-SiRNA triggers of 21-25 nucleotides have been used to elicit RNA interference in mammalian cells (Caplen, 2001; Elbashir, 2001 a). A rationale for choosing these lengths was based on the observation that long double-stranded RNAs are cleaved into 21-22 nucleotide fragments that serve as intermediates for the interference process (Elbashir, 2001 b). The effect of RNAi triggered by ds-SiRNA triggers consisting of 14, 17, and 19 nucleotides in each strand was investigated (FIG. 45 and 46A and B). Again, these triggers were designed to have 3′ extensions of two dT residues that do not base pair with the target mRNA (in the case of the antisense strand). Hence, target homologies of these triggers were 12, 15, and 17 nucleotides, respectively. As shown in FIG. 46 (A, bars, 1-3), a ds-SiRNA with 19 nucleotides in each strand was effective in triggering the RNAi response, indicating that ds-SiRNA triggers with a 17 nucleotide homology to the target strand may work effectively. However, as shown in FIG. 46 (A and B), certain ds-SiRNA with 17 nucleotides in each strand were not effective in triggering RNAi. There may be several reasons for the inability of ds-SiRNA with 17 nucleotides in each strand to trigger RNAi in this work. One potential reason might be that the length of the duplex is too short to assemble into RiSC upon binding to a number of proteins. Another potential reason could be based on insufficient homology for the RNA interference process to work here.

To identify which factor might be important for RNA interference, three more ds-SiRNA triggers were designed to differentiate between the homology and the length of the trigger (FIG. 46, B). The length of the triggers was left at 21 nucleotides, but the length of the homology was 17, 15, and 12 nucleotides, respectively (FIG. 45, sequences 4, 5 and 6). The ds-SiRNA triggers of 21 nucleotide length, that also carried either a 15 or 17 nucleotide stretch homologous to the targeted mRNA were effective in RNA interference. The trigger with only a 12 nucleotide homology was not effective (FIG. 46, B, Bar 6). These results suggest that both the length of the ds-SiRNA trigger and the number of nucleotides in the homologous region may play a role in certain RNAi processes.

Nuclease Resistant SiRNA Triggers

Both ss- and ds-SiRNA triggers (unimolecular and bimolecular) that are stable to nucleases, and hence may be effective in initiating and maintaining an RNAi response in cells, were studied. Nuclease stable SiRNA triggers may survive longer in certain biological fluids both during and after their delivery into the cells. Once inside the cell, mere resistance to degradation may make nuclease stable triggers last longer, thereby establishing long lasting RNA interference. Moreover, certain modifications may facilitate cell penetration. The following modifications in either antisense or sense strand were made to explore the possibilities for creating chemically modified SiRNA triggers (FIG. 47).

1. Inverted abasic residue at the 3′ end after two dT residues.

2. Inverted dT residue at the 3′ end after two dT residues.

3. 2′-F pyrimidines.

4. 2′-OMe in all nucleotides.

5. DNA.

The inverted abasic residue and the inverted dT residue may function like 3′ caps that further protect an oligonucleotide from 3′-5′ exonucleases, in addition to the protection provided by two dT residues. Replacement of the 2′-OH group in the sugar by various groups such as NH₂, F and OMe may make RNA more nuclease resistant (Lin et al., 1994; Pieken et al, 1991). As shown in FIG. 47, both modifications at the 3′ end do not appear to interfere with the RNAi mechanism, suggesting that SiRNA triggers that are protected from 3′-5′ exonuclease may be designed. Substitution of all pyrimidines by 2′-F pyrimidines in both strands may also be tolerated by the RNAi mechanism. It has been shown previously that the 2′-F pyrimidine substitution alone may be sufficient to make RNA more nuclease resistant and the half-life of such modified RNA molecules may be increased significantly (Green et al., 1995; Lin et al., 1994; Pagratis et al., 1997). Based on these previous studies, SiRNA triggers carrying all pyrimidines with the 2′-F modifications in combination with 3′- and 5′-cap may be significantly nuclease resistant and may be effective in triggering RNAi. Additionally, the 5′-caps could have any modification that will substantially prevent or reduce exonuclease attack. Certain examples of modifications that may be used are dT, or modifications with amino, cholesterol, thiol groups, or modifications with a dye molecule such as fluoroscein, or modifications with polyethylene glycol, a lipid, or a carrier peptide.

Ds-SiRNA triggers with one strand with a 2′-OMe substitution inhibited the RNAi process. However, it may be possible to have SiRNA triggers modified with 2′-OMe either in pyrimidines, or in purines, or at specific sites. SiRNA triggers with a single DNA strand may be analogous to SiRNA triggers with a 2′-OMe modification. RNA-DNA hybrids were not efficient in eliciting the RNAi process in this work. The lower level of RNAi in mammalian cells by SiRNA with DNA strands, as demonstrated here, is in agreement with a previous observation made in C. elegans with long RNA-DNA hybrid molecules. Previously, using relatively long dsRNA molecules Parrish, et al. observed the tolerance of 2′-Furacil in RNA interference in C. elegans (Parrish, 2000).

Next, a unimolecular trigger based on the fold-back hairpin structure was generated with 2′-F pyrimidines throughout the molecule and with a single inverted dT at the 3′ end (FIG. 48). As shown in FIG. 49, the chemically modified unimolecular ss-SiRNA trigger may be effective in silencing the transient expression of SEAP. This observation supports the notion that unimolecular ss-SiRNA triggers may be optimal for certain RNAi. As presented in FIG. 49, the use of a unimolecular trigger may be more effective than a bimolecular (ds-SiRNA) trigger to elicit RNAi by targeting an identical site within the target mRNA (FIG. 49, compare Sphp-2 and Seap 2).

The successful use of SiRNA triggers including both pyrimidine nucleotides carrying a 2′-F modification either on both strands (in the case of bimolecular ds-SiRNAs) or throughout the molecule (in the case of unimolecular ss-SiRNAs) was observed. Taken together, these results suggest that the design of SiRNA triggers carrying specific chemical modifications may be a viable approach to initiate RNA interference in mammalian cells. Certain additional modifications to SiRNA triggers include, but are not limited to, the addition of polyethylene glycol, the addition of lipids, and the addition of dyes, such as flourescein. It may be that these triggers will pave the way for creating effective SiRNA triggers for clinical applications.

### CONCLUSIONS

RNA Interference Compared to Antisense

1. Relatively low or almost no toxicity to the cells transfected with SiRNA triggers was observed, whether they were ds-SiRNA or ss-SiRNA that form short hairpins, compared to the single stranded antisense molecules. Transfection with SiRNA triggers in this work did not lead to the reduction of nonspecific mRNAs. In certain instances, this may make RNAi technology more versatile than antisense techniques in general, allowing researchers to transfect cells at low cell density. This also suggests that RNA interference may be a useful tool to study cellular pathways that are sensitive to external insults.

2. RNAi in general may be more potent than antisense; about 10-fold lower concentration of SiRNA compared to the concentration of antisense oligonucleotides gave approximately the same level of mRNA reduction in this work. Effective RNA interference with SiRNA triggers transfected at 5 nM was observed.

3. Similar to antisense oligonucleotides, in certain instances, the efficiency of SiRNA triggers may be target site dependent, suggesting that targeting certain sites in a target mRNA may not be effective in eliciting gene silencing through RNAI.

4. SiRNA triggers may be picked randomly without any help from a computer algorithm. Since targeting to certain sites on mRNA may not work, a handful of triggers (between 3 and 5) may be tested. This is in contrast to the antisense approach that often involves screening 30-40 oligonucleotides. Since the conversion of a ds-SiRNA trigger that was not effective into a unimolecular trigger that produced effective interference was observed, it may be that the number of unimolecular triggers that are screened may be even lower.

Certain Novel SiRNA Triggers

1. Effective unimolecular SiRNA triggers that are single-stranded with the propensity to form short hairpins were observed. In those ss-SiRNA HP triggers, the nature of the nucleotides in the loop may not affect the efficiency of RNA interference.

2. Certain ss-SiRNA HP triggers appear to be sensitive to nucleotide deletions.

3. In certain embodiments, unimolecular siRNA triggers may contain synthetic loops. In certain embodiments, unimolecular siRNA triggers may contain polyethylene glycol loops.

Nuclease Stable SIRNA Triggers

1. The incorporation of a nuclease resistant cap, such as inverted dT and inverted abasic residues at the 3′ end of both strands of a ds-SiRNA trigger, may be effective in RNA interference. This also may work in ss-SiRNA triggers. Furthermore, the same strategy may work at the 5′ end as well.

2. In certain instances, pyrimidines in both strands of ds-SiRNA trigger may be replaced by 2′-F modified pyrimidines, making such triggers resistant to nucleases.

3. In the case of certain unimolecular ss-SiRNA triggers, the 2′-F modification may be introduced throughout the sequence in all pyrimidines without substantially compromising the efficacy of RNA interference.

Features of Certain SiRNA Triggers

1. Ds-SiRNA triggers may be designed to carry 3′ extensions made up of two dT residues that do not recognize the target. In other words, target sites on an mRNA may be any sequence, not restricted to the nature of N₁₉-AA. This opens up a broad range of target sites within a target gene.

2. In certain instances, the length of each strand within a ds-SiRNA may be as short as 19 nucleotides out of which 17 nucleotides form a contiguous stretch of target homology with two nonhomologous dT residues at the 3′ extensions.

3. In certain instances, the region of homology may be as short as 15 nucleotides when the length of the trigger is 21 nucleotides. A 19 nucleotide long ds-SiRNA harboring 15 nucleotides of target homology may be effective in RNA interference.

4. In certain instances, ds-SiRNA triggers may tolerate a single base pair mutation.

5. In certain instances, mutations in the sense strand of the ds-SiRNA trigger also affect the efficiency of RNA interference. In certain instances, ds-SiRNA triggers with more than two mutations in the sense strand may decrease the RNAi effect. In certain instances, the effectiveness of RNA interference may vary depending on the mutation site in the sense strand, such that an effect may be observed when the mutation is located in the middle, as opposed to near the end of the RNA.

II. Gene Identification and Functional Analysis Using Short Interfering Random Sequence (SiRS)RNA Hairpin Libraries

Identification and characterization of novel genes combined with the functional analysis of identified genes may enable the discovery of novel drug targets. With the completion of the human genome database, companies are racing to identify novel genes. Among these may be genes that could be linked to various diseases.

Some technologies exist to potentially identify a function of a gene based on sequence information. Antisense oligonucleotides upon pairing-up with a known mRNA sequence mediate the destruction of a message in a cell, leading to the inactivation of gene expression. This technique may be aided by prior knowledge of the mRNA sequence of a gene and may be used to validate gene function. On the other hand, there are many genes in the human genome database whose sequences are not known to date. Furthermore, it may be that alternate splicing will lead to the generation of many splice variants to generate different protein products. This may further complicate the gene identification process.

A reverse genetic strategy to identify genes exclusively based on their function may be possible, allowing discovery of new genes from the human genome as well as the analysis of the functions of known genes. Furthermore, the proposed strategy may also help identify novel pathways of complex biological processes. This strategy uses epigenetic interference of gene expression using double-stranded RNA (dsRNA) molecules. In this work, certain double stranded RNA (dsRNA) corresponding to a sense and antisense sequence of an mRNA was introduced into a cell, the corresponding mRNA was degraded and the gene was silenced. This post-transcriptional gene silencing phenomenon, mediated by double stranded RNA sequences, is commonly referred to as RNA interference or RNAi (Fire, 1998). RNAi has been observed in a wide range of organisms, including nematodes, insects, trypanosomes, planaria, hydra, zebrafish, and the mouse (Reviewed in Bosher, 2000; Hammond, 2001). RNAi may be functionally related to posttranscriptional gene silencing observed in plants and quelling observed in Neurospora crassa. Since double stranded RNA mediates all three processes, they can be collectively called “RNA silencing” (Voinnet, 2001; Waterhouse, 2001).

Biochemical studies carried out with a Drosophila in vitro system led to the discovery of short RNA duplexes (21-22-nucleotides) that could effectively trigger gene silencing through RNAi (Elbashir, 2001 b; Yang, 2000; Zamore, 2000). Recently, this epigenetic interference of gene silencing by short interfering RNAs (SiRNAs) has been demonstrated in cultured cells of mammals (Caplen, 2001; Elbashir, 2001a), opening the door for using RNAi technology in characterizing the human genome. Although complete details of the cellular mechanism that initiates and propagates gene silencing through RNA interference is unknown, it may be an extremely specific and very potent mechanism to inhibit gene expression by degrading specific mRNA. Only a few molecules of dsRNAi triggers per cell may be needed to inhibit mRNA present at high concentrations, suggesting an inherent amplification step present in the RNAi process. In certain instances, its specificity, efficacy and generality offer an attractive approach for studying gene function.

Short Interfering Random Sequence (SIRS)RNA Hairpin Libraries

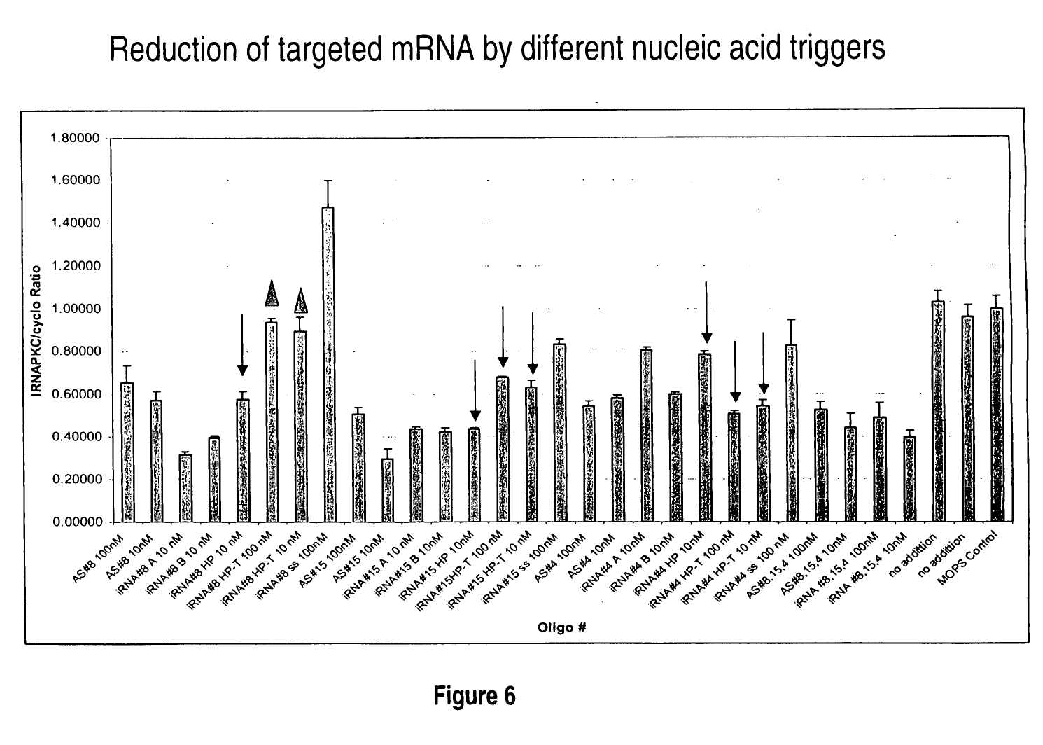

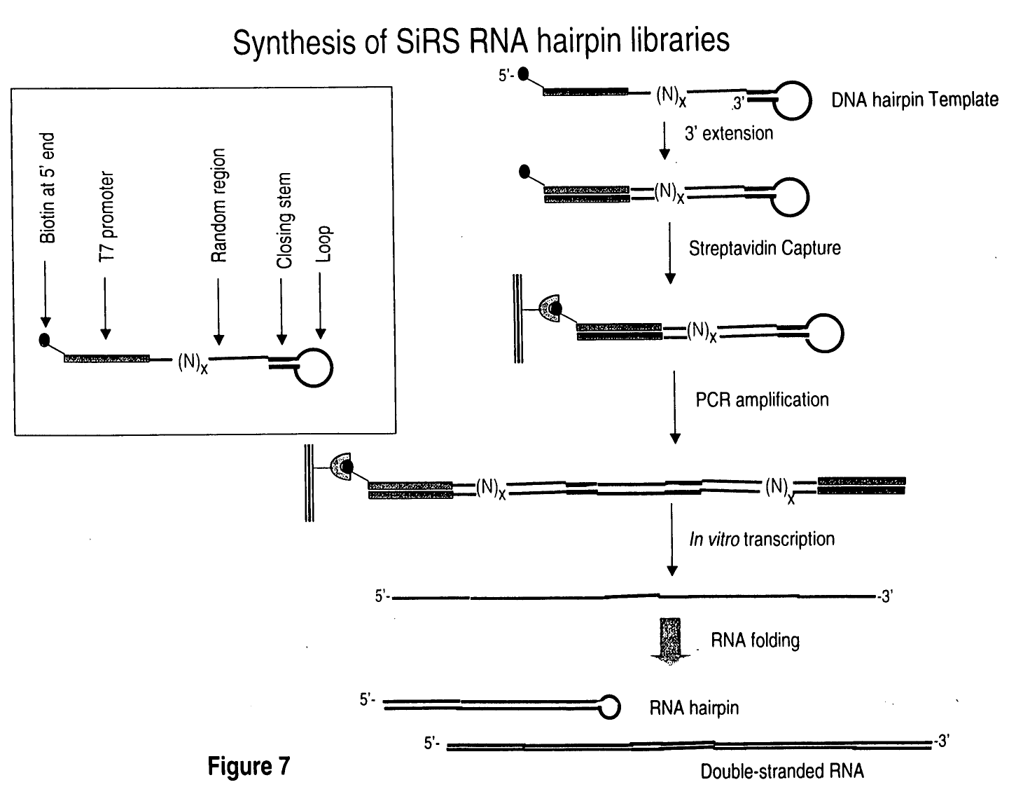

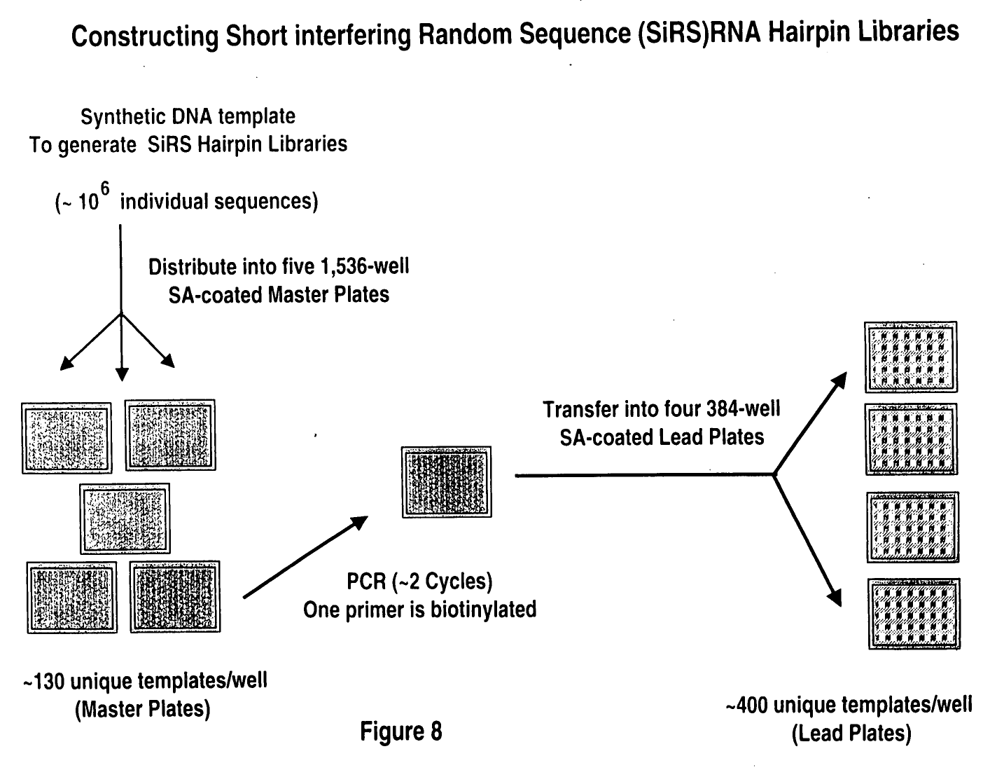

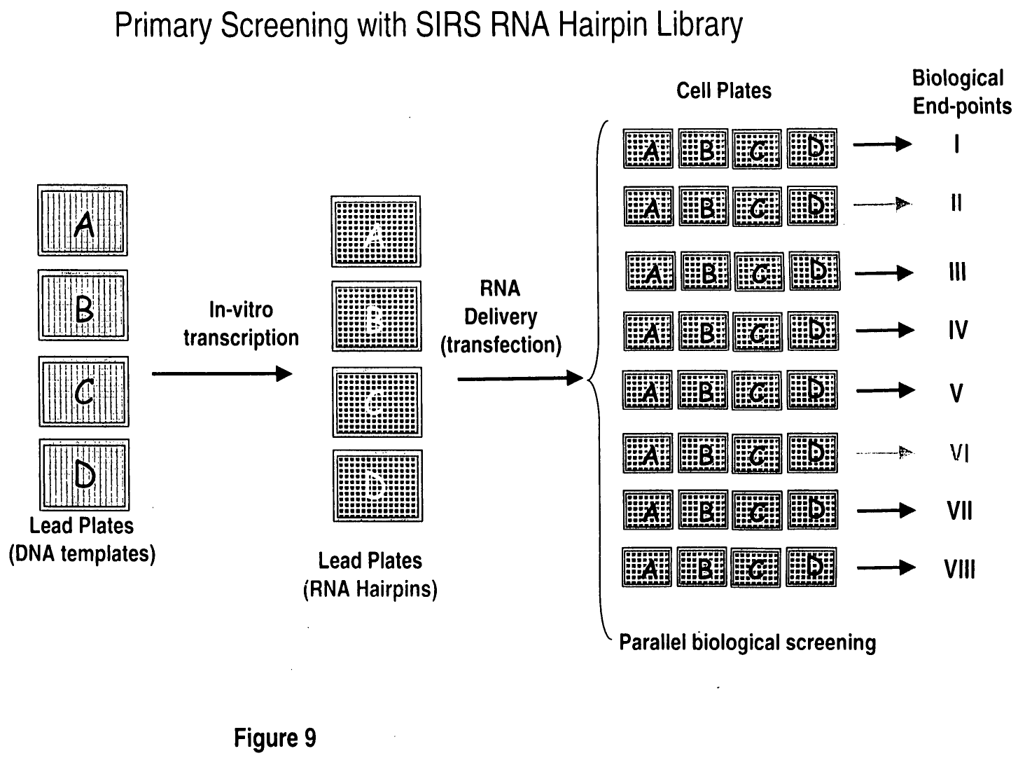

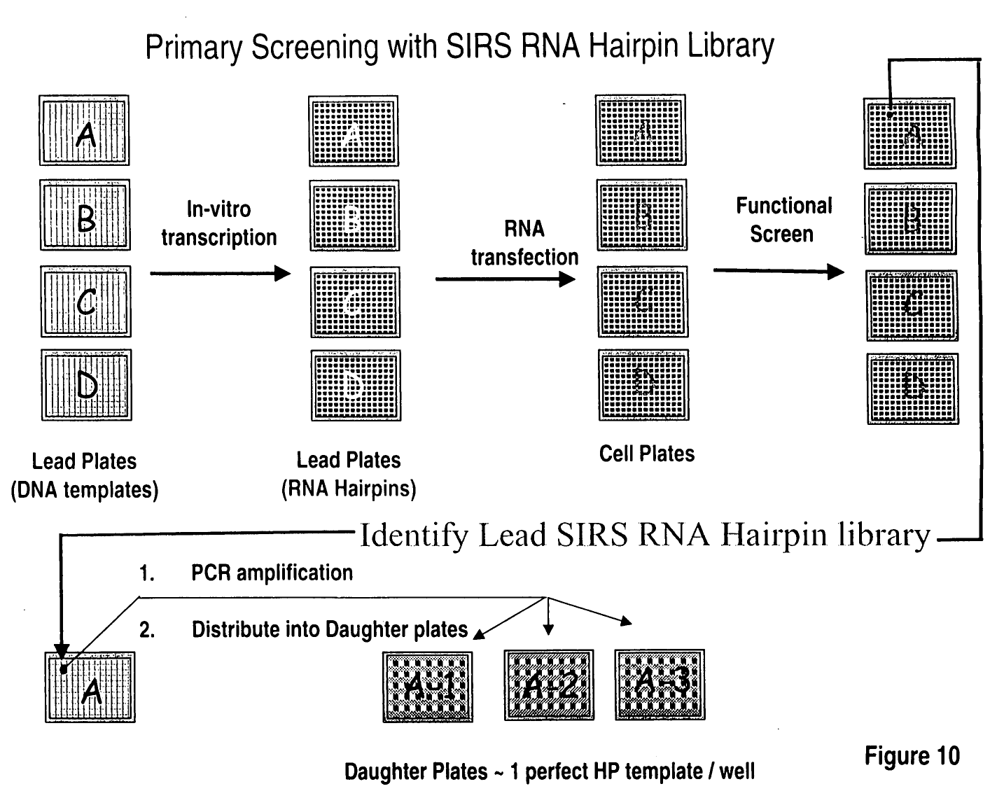

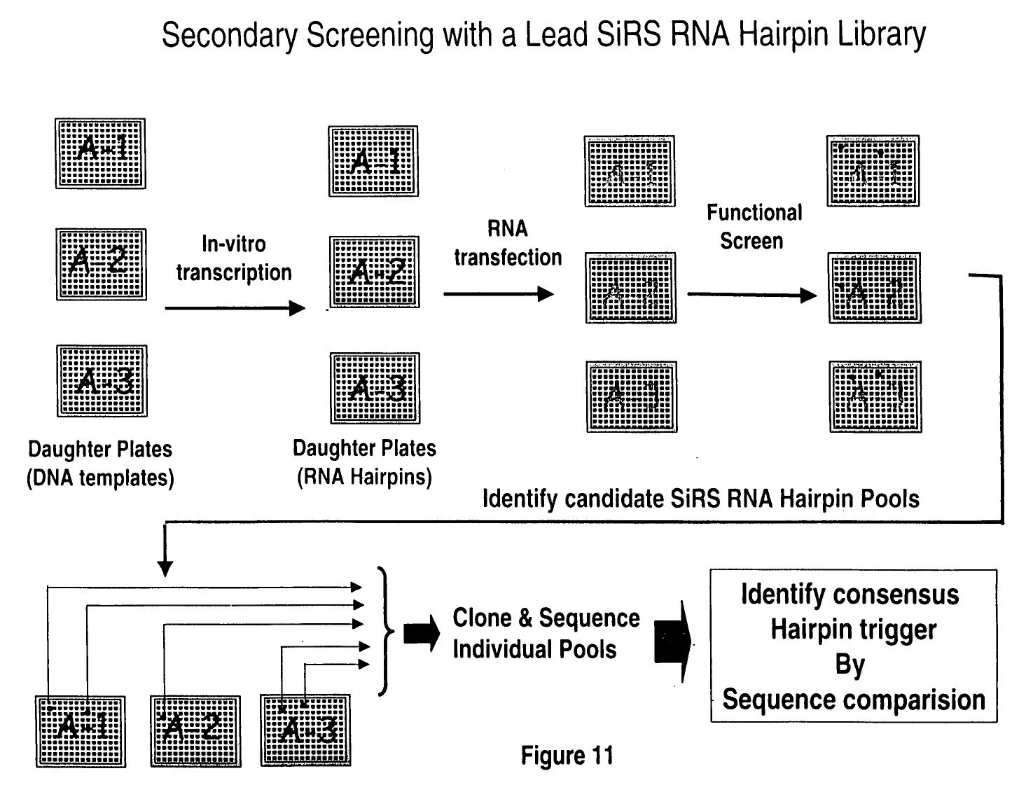

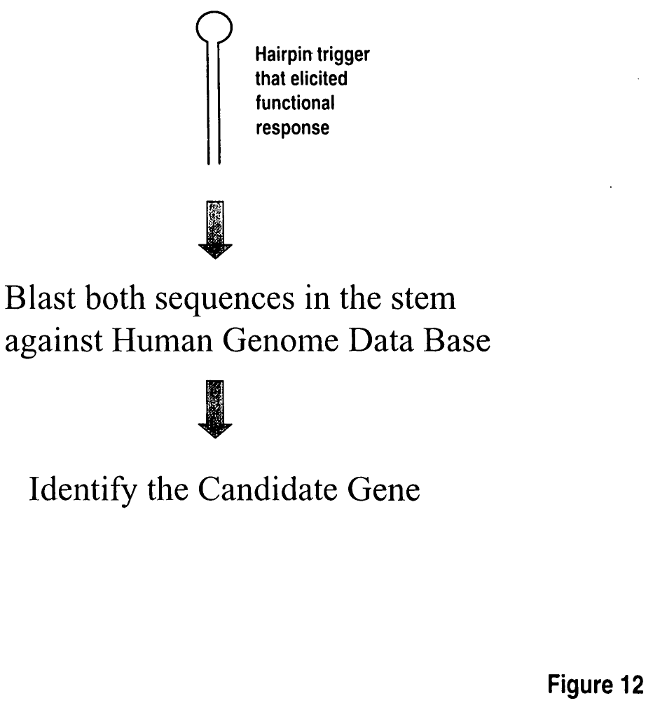

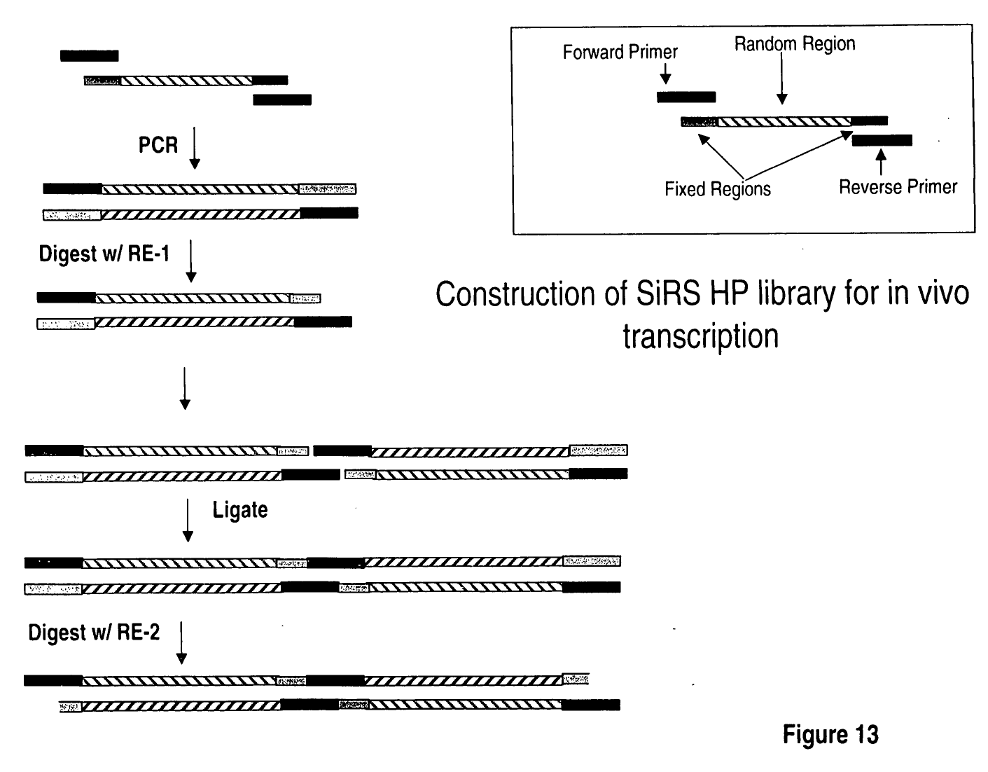

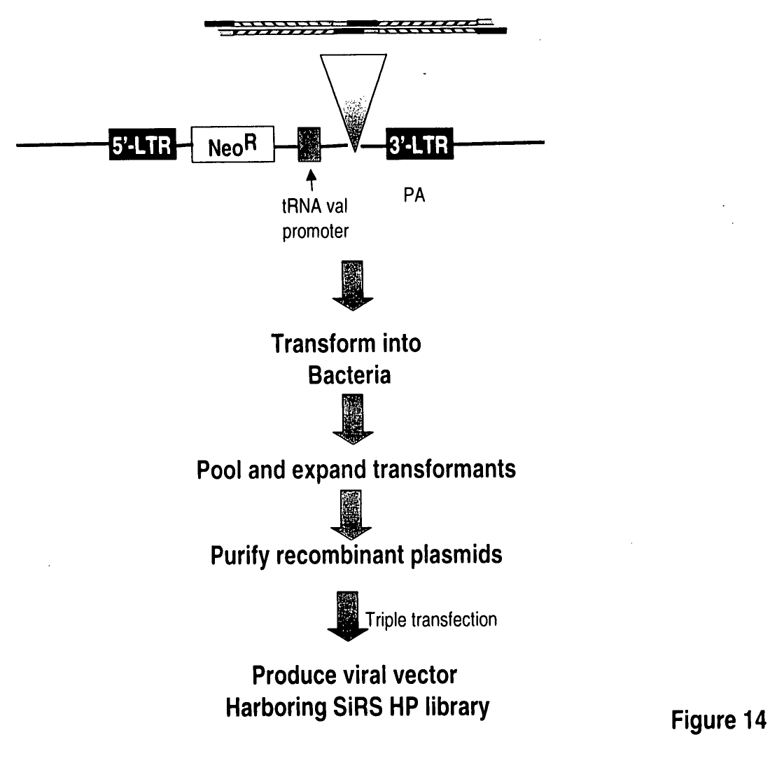

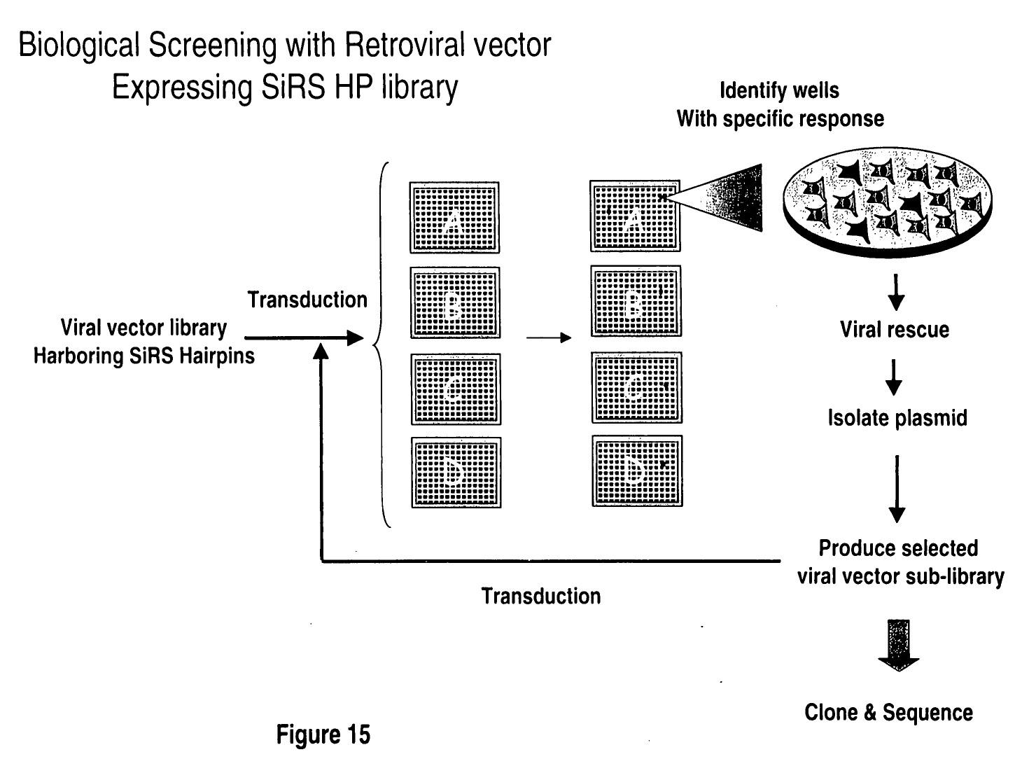

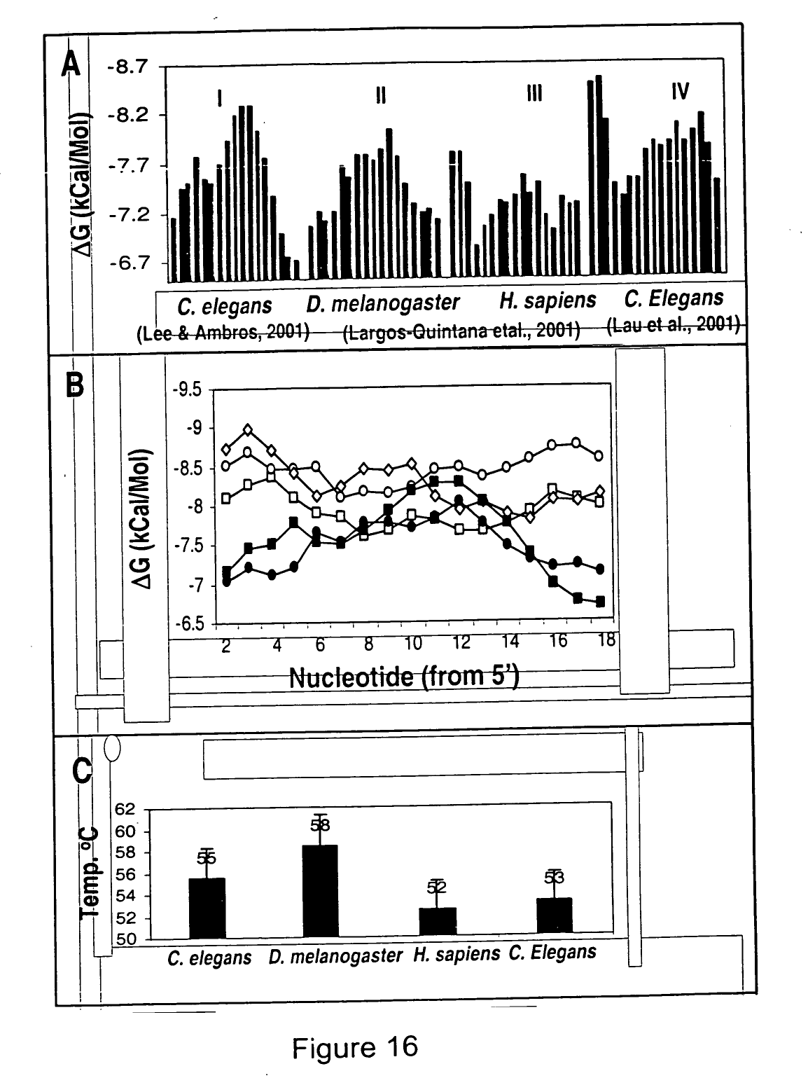

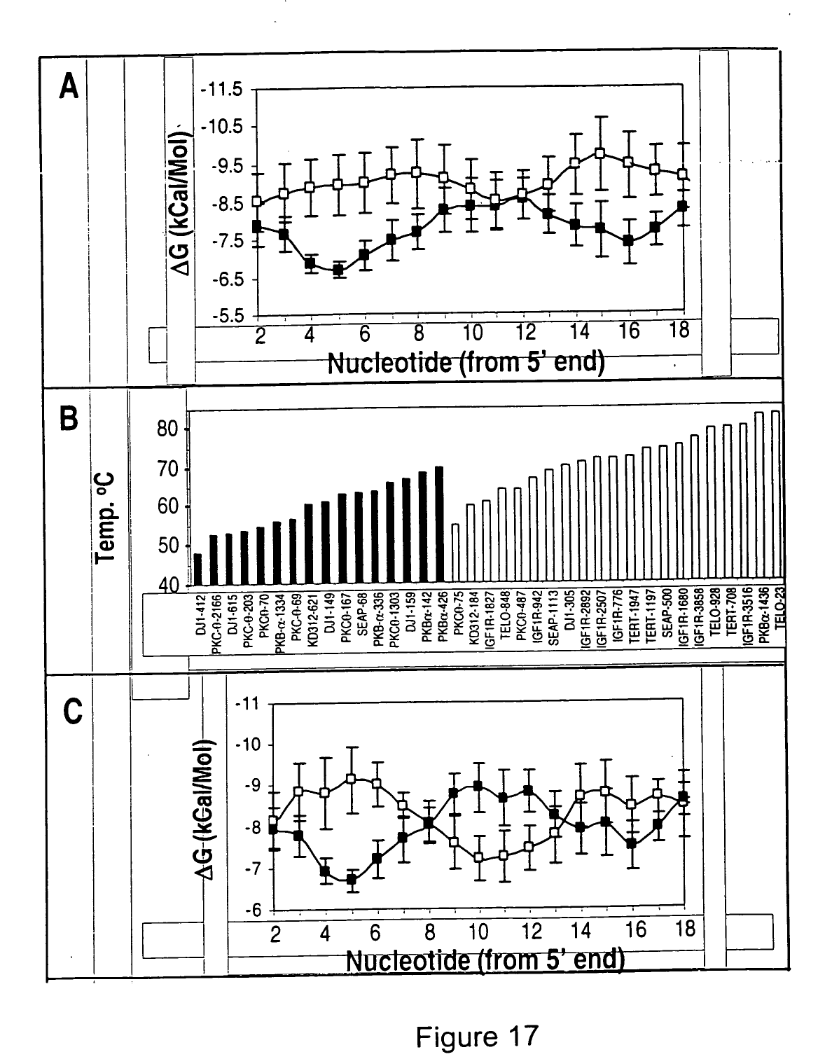

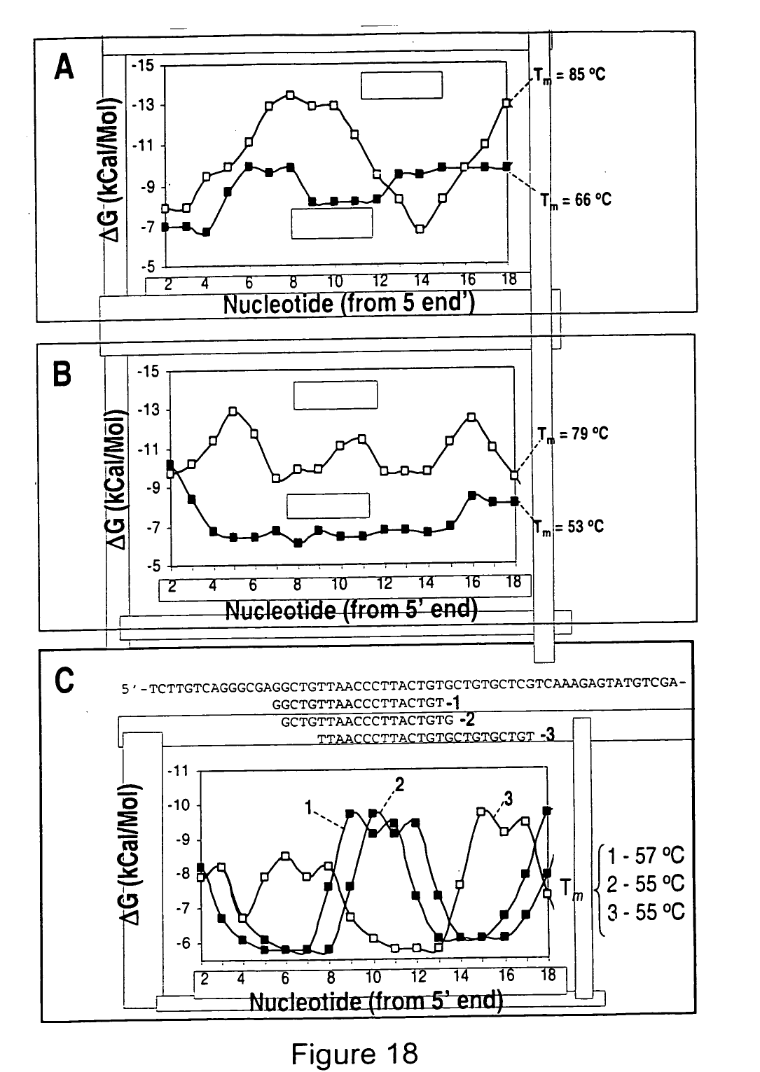

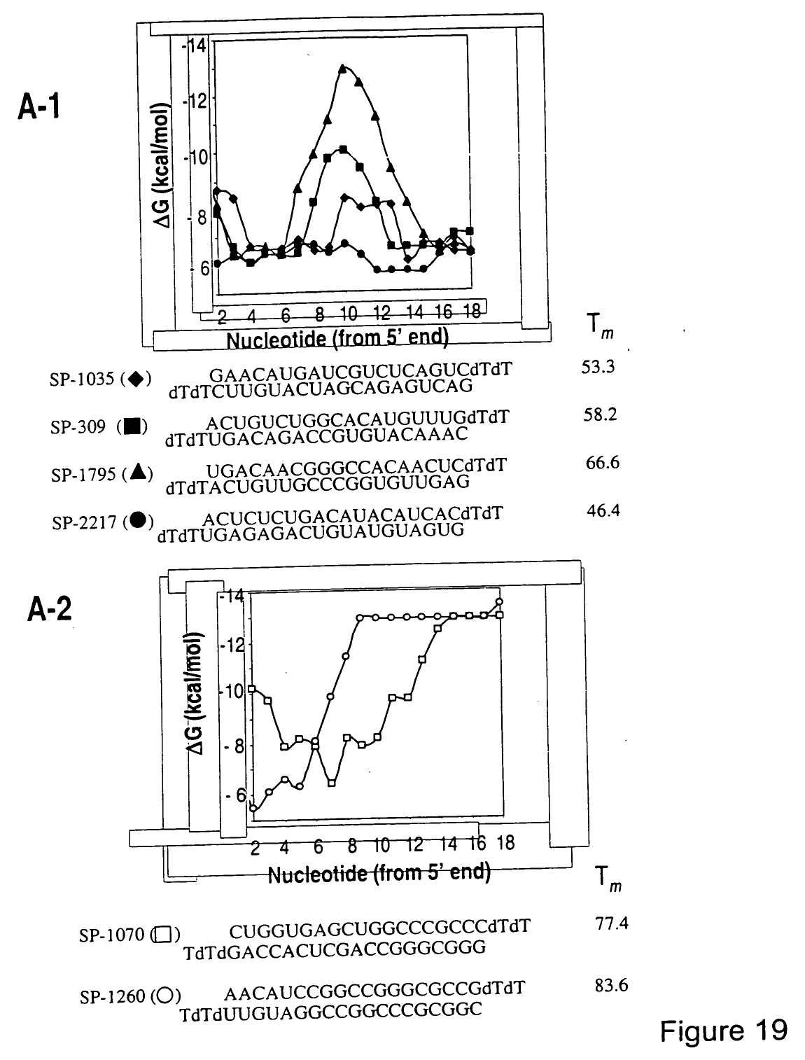

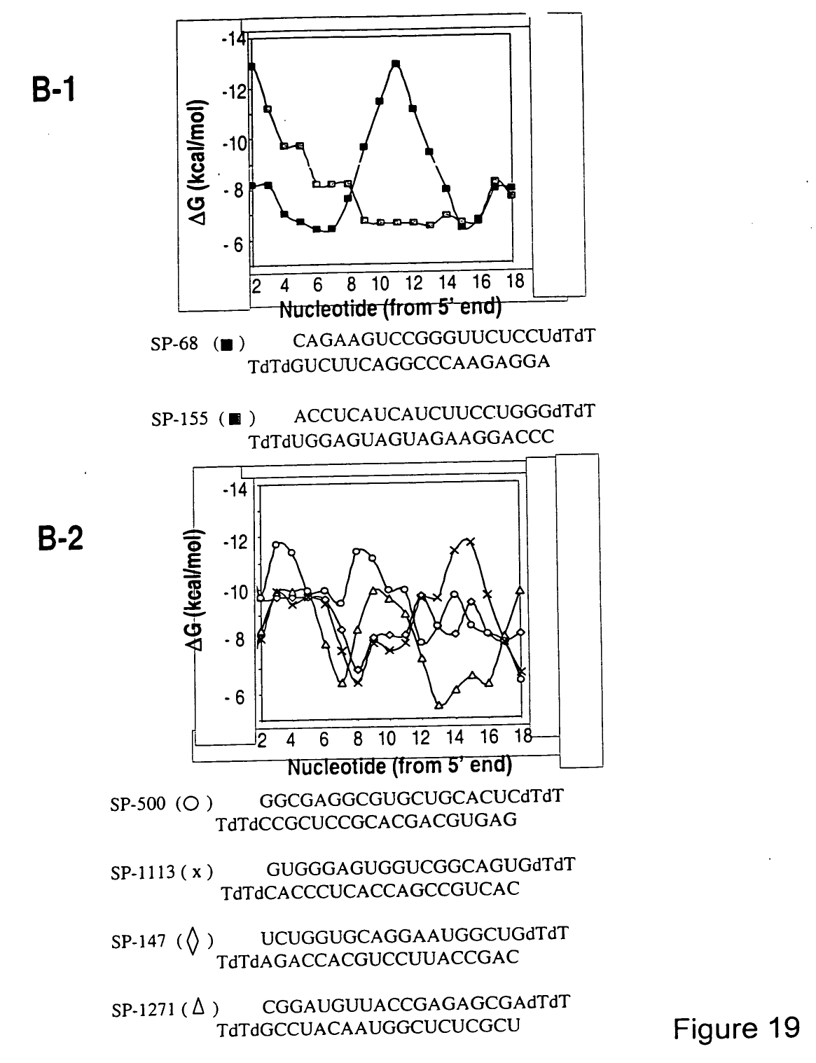

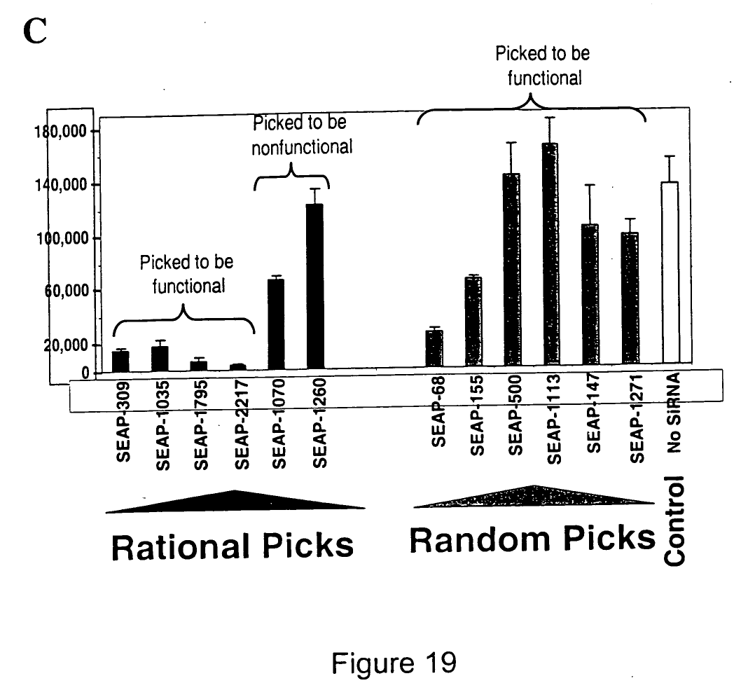

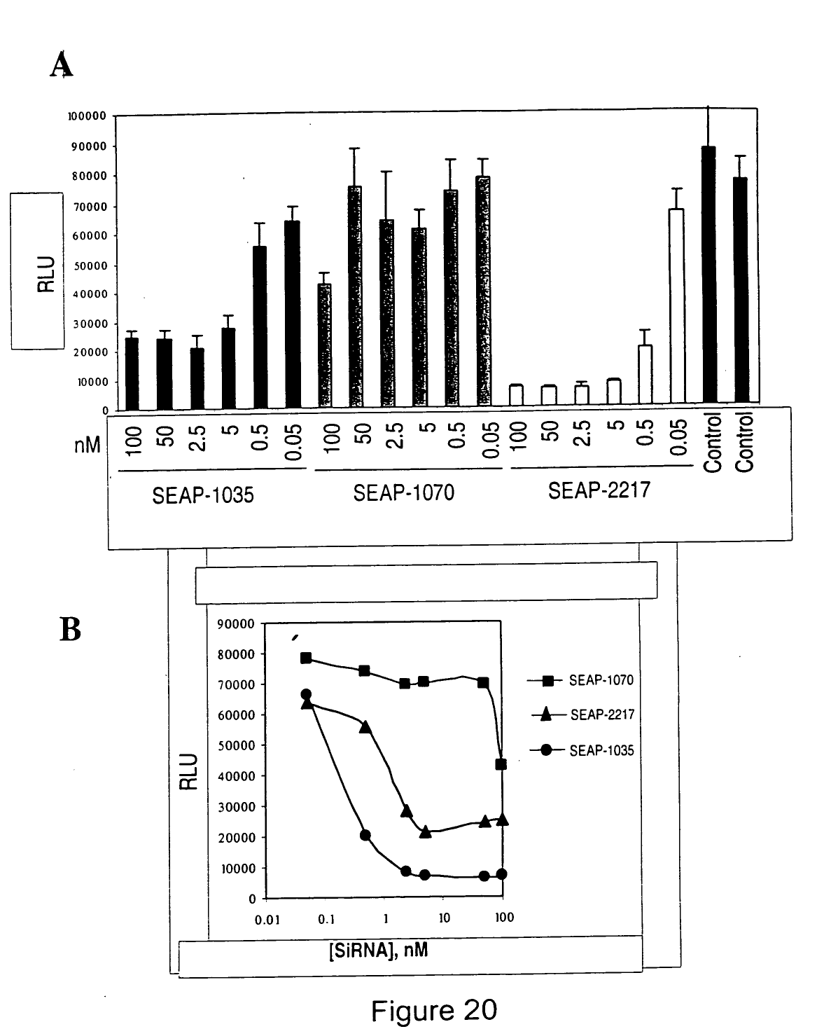

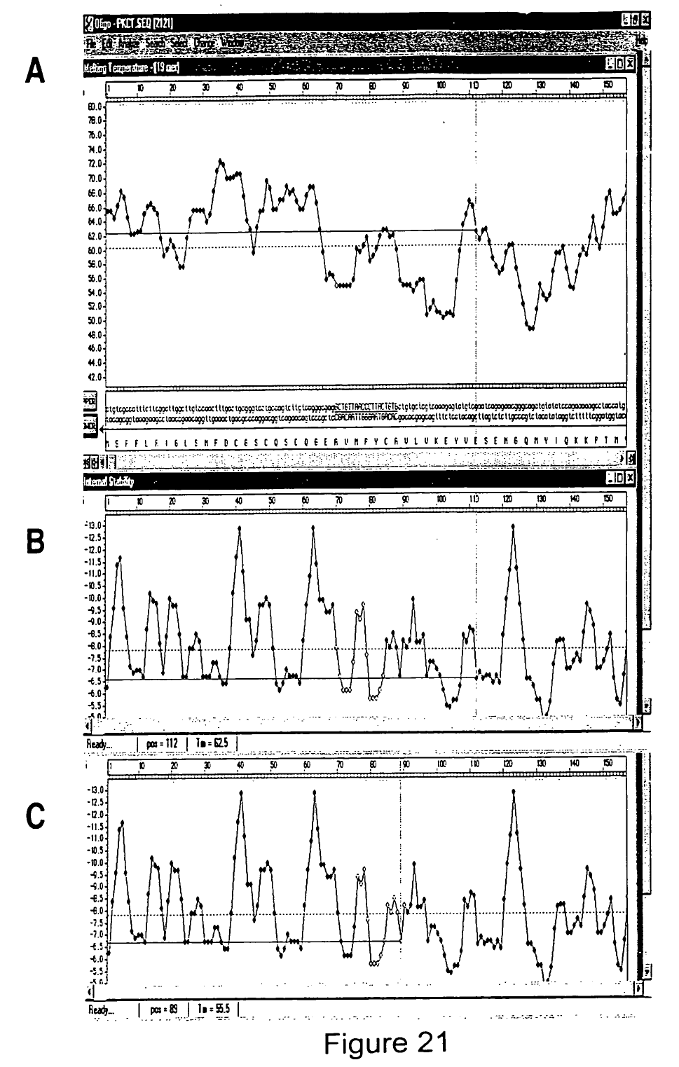

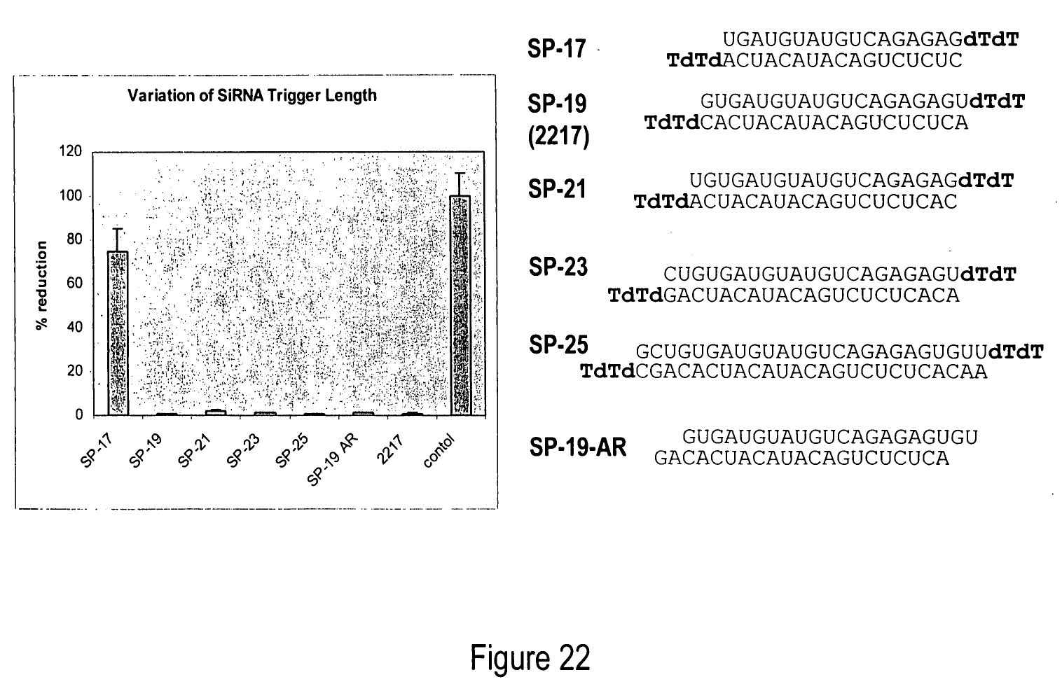

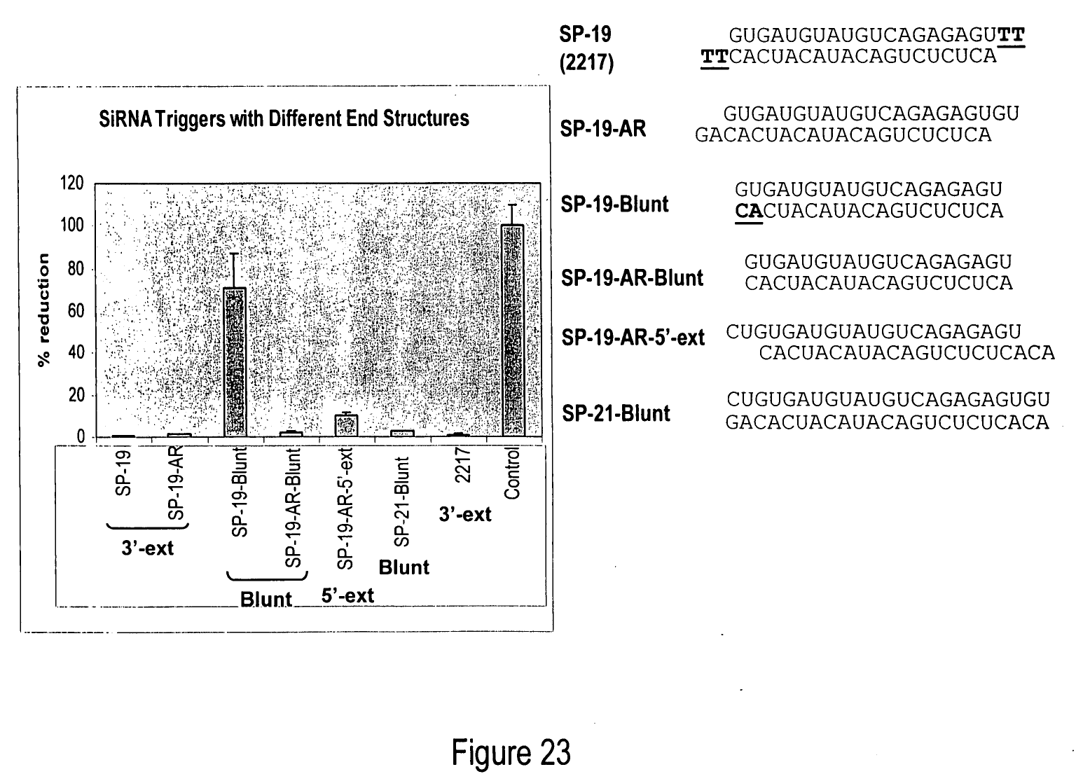

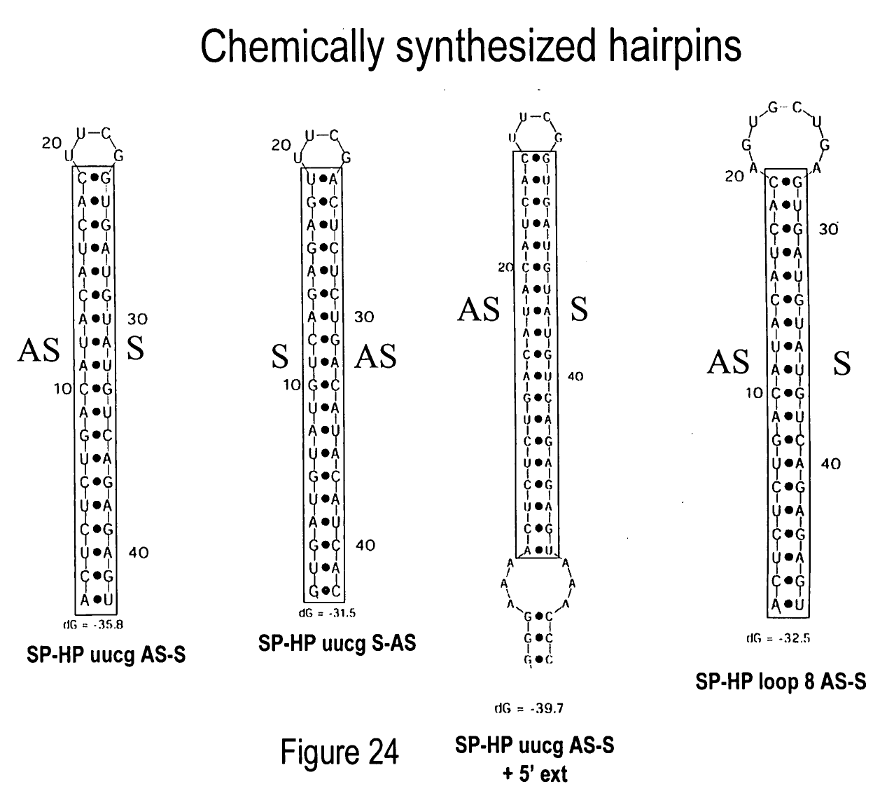

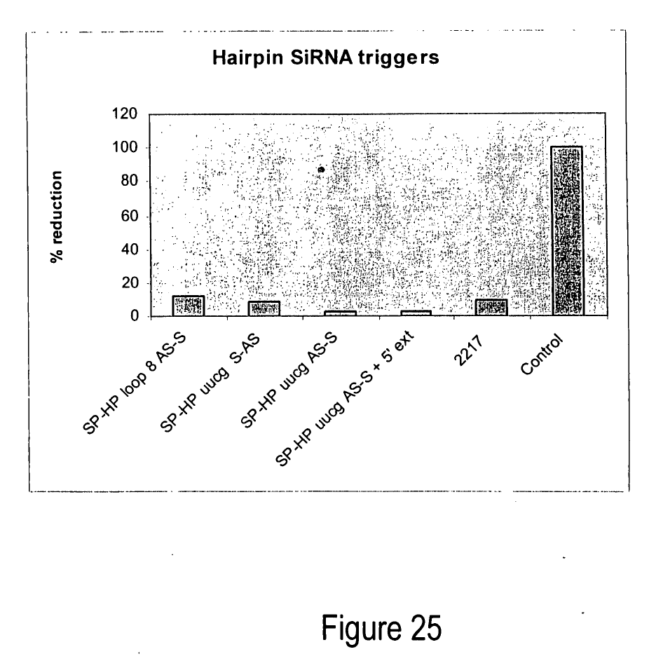

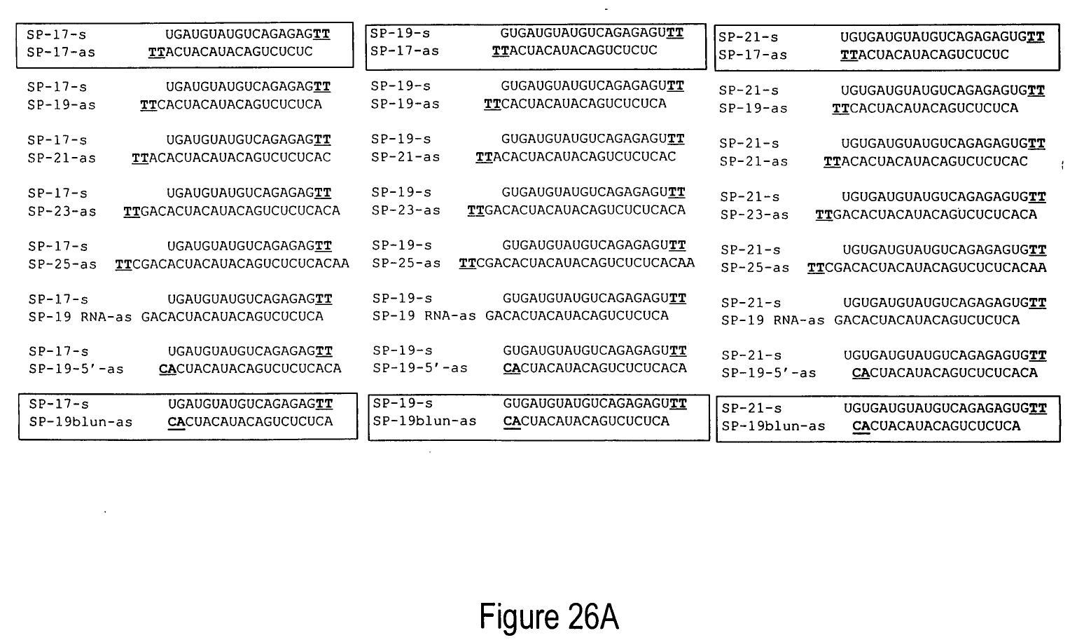

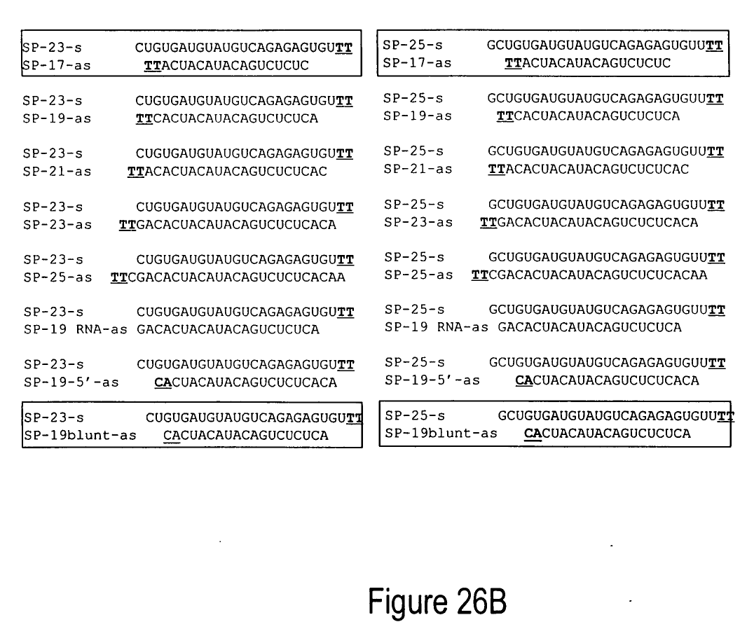

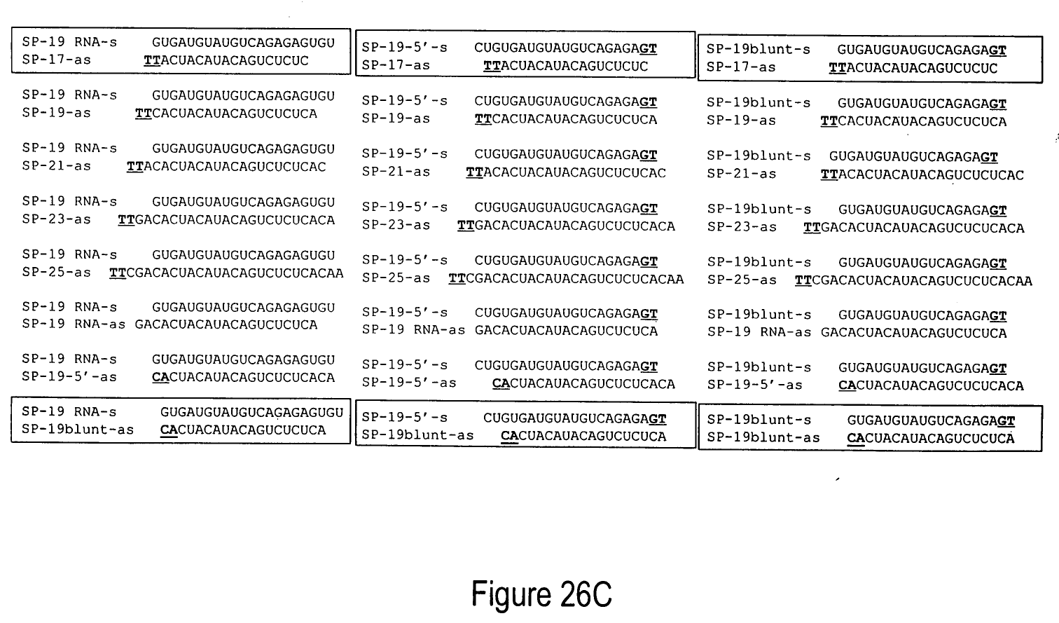

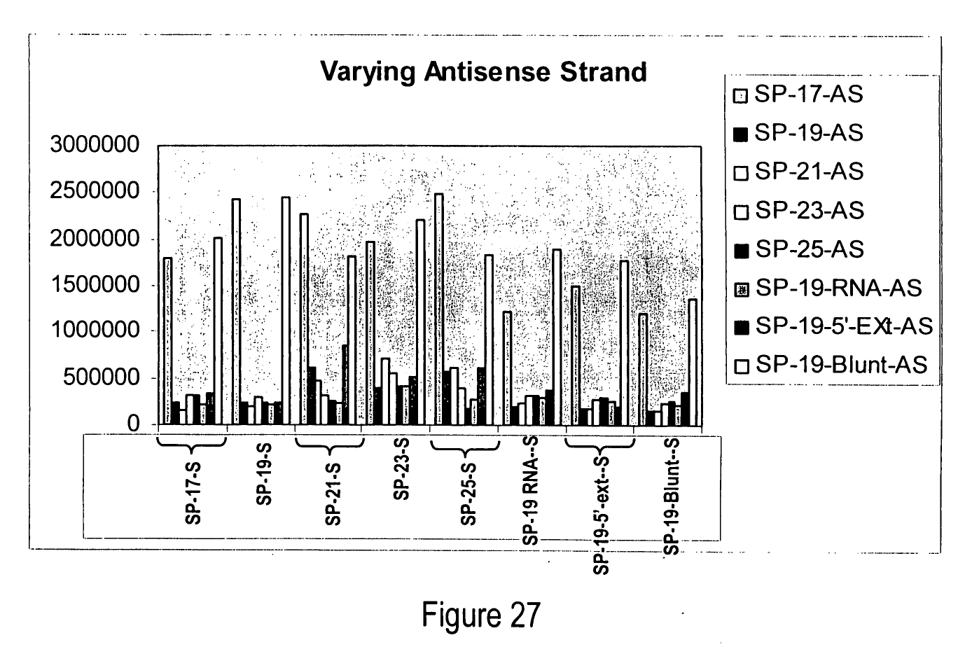

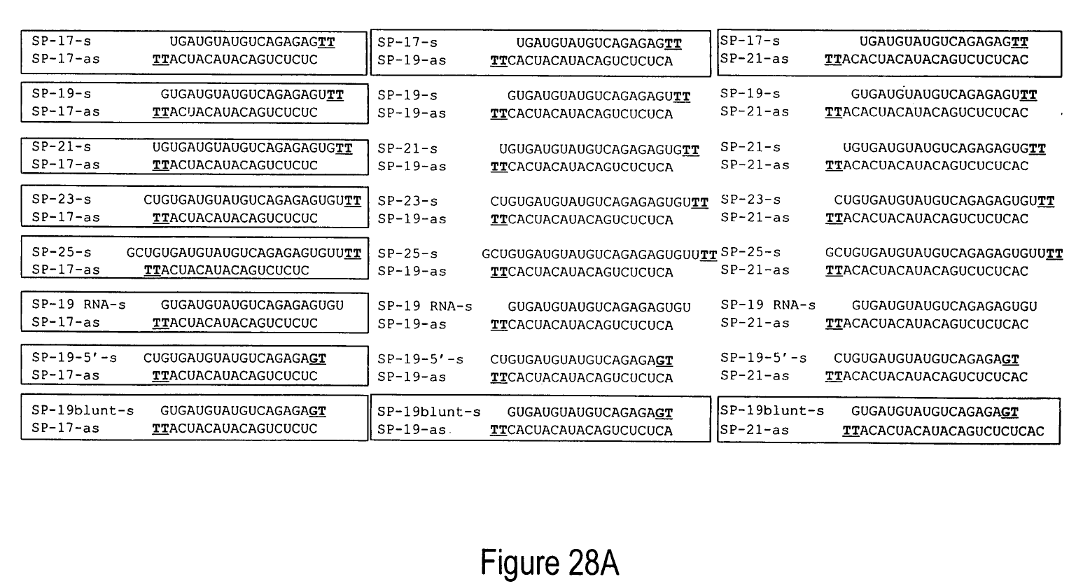

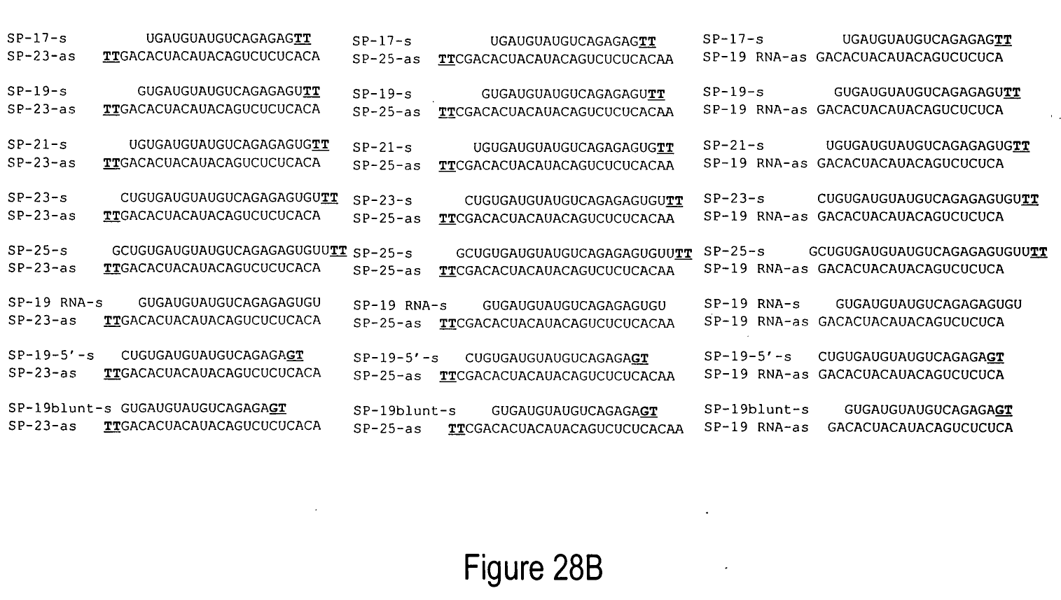

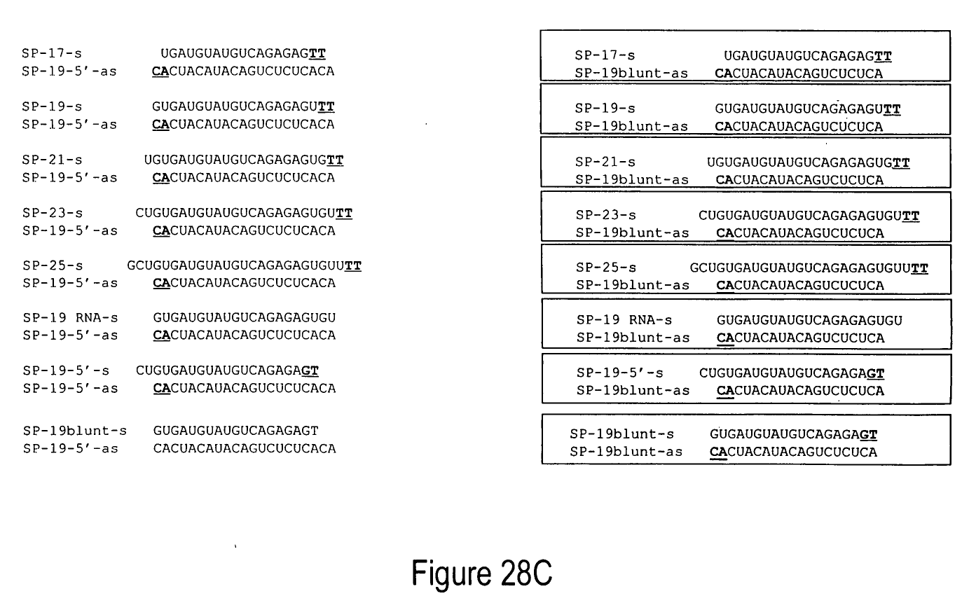

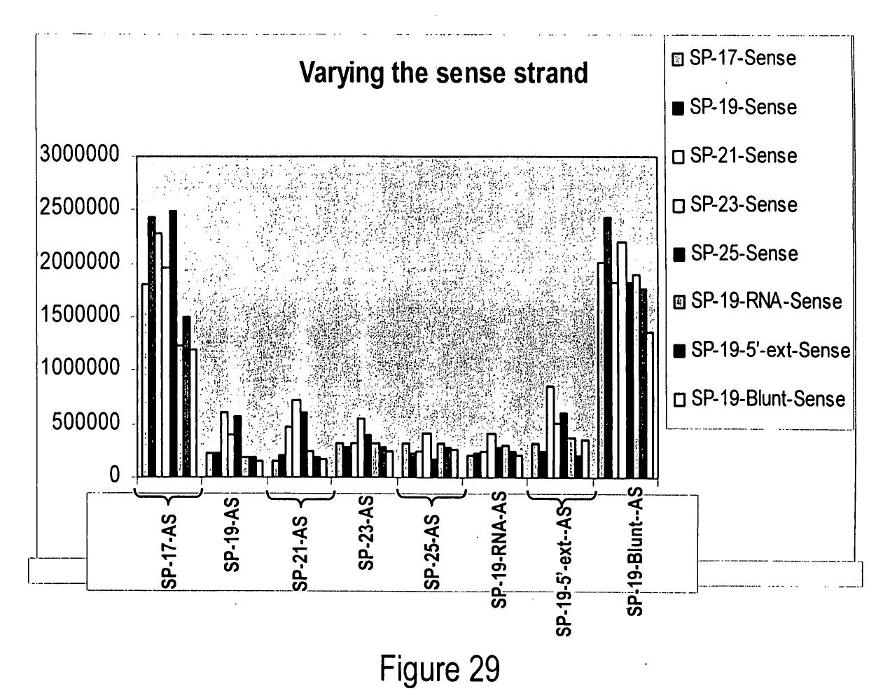

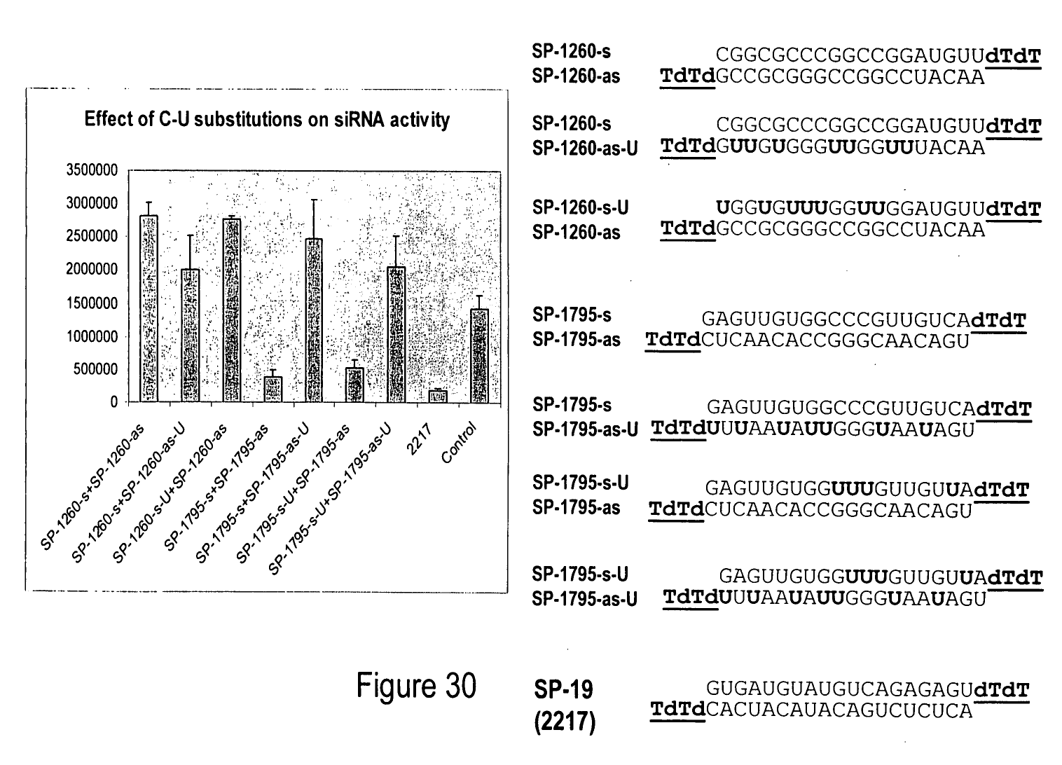

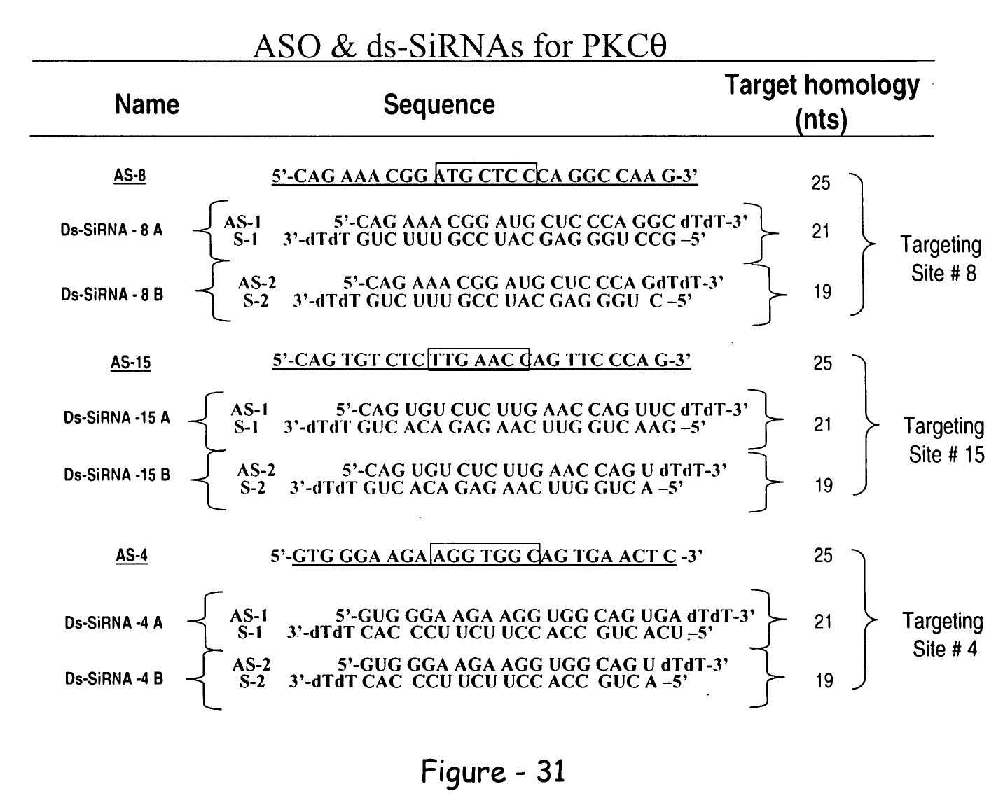

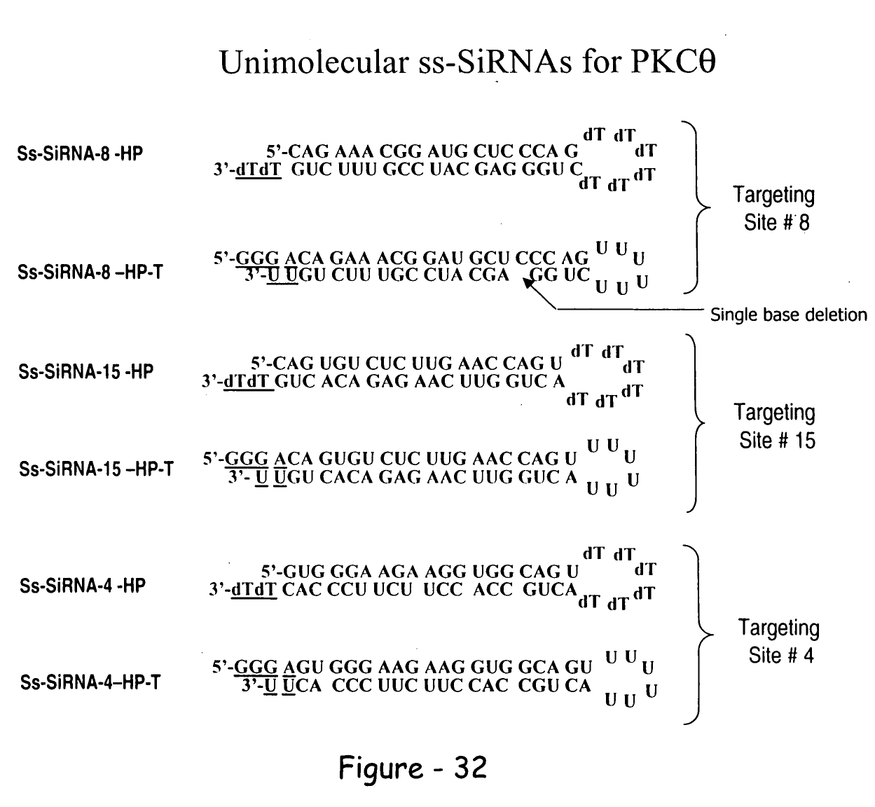

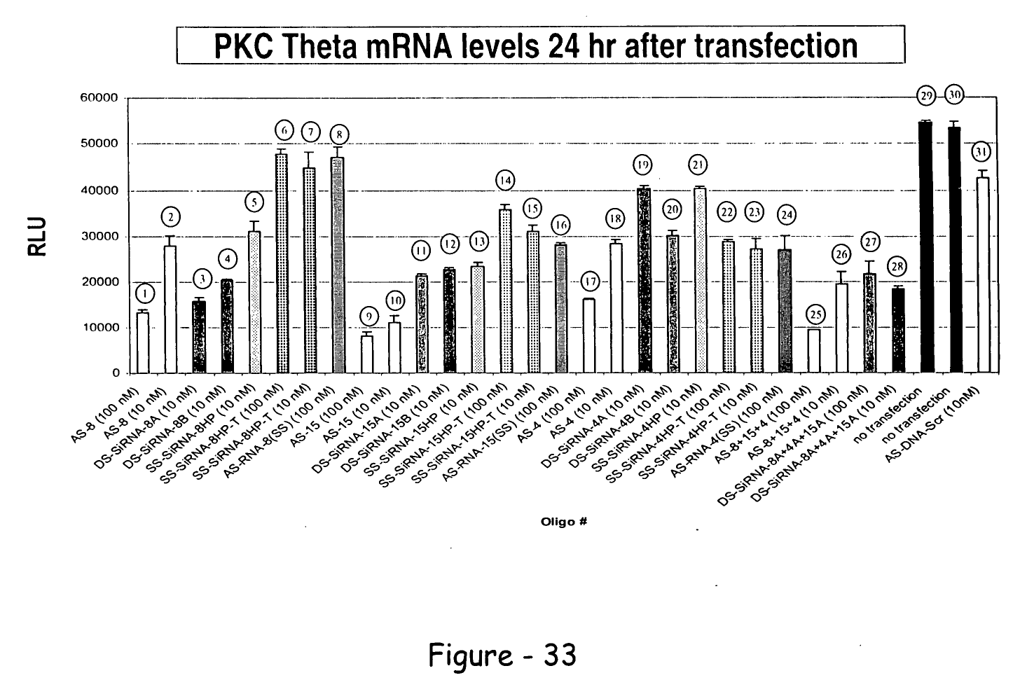

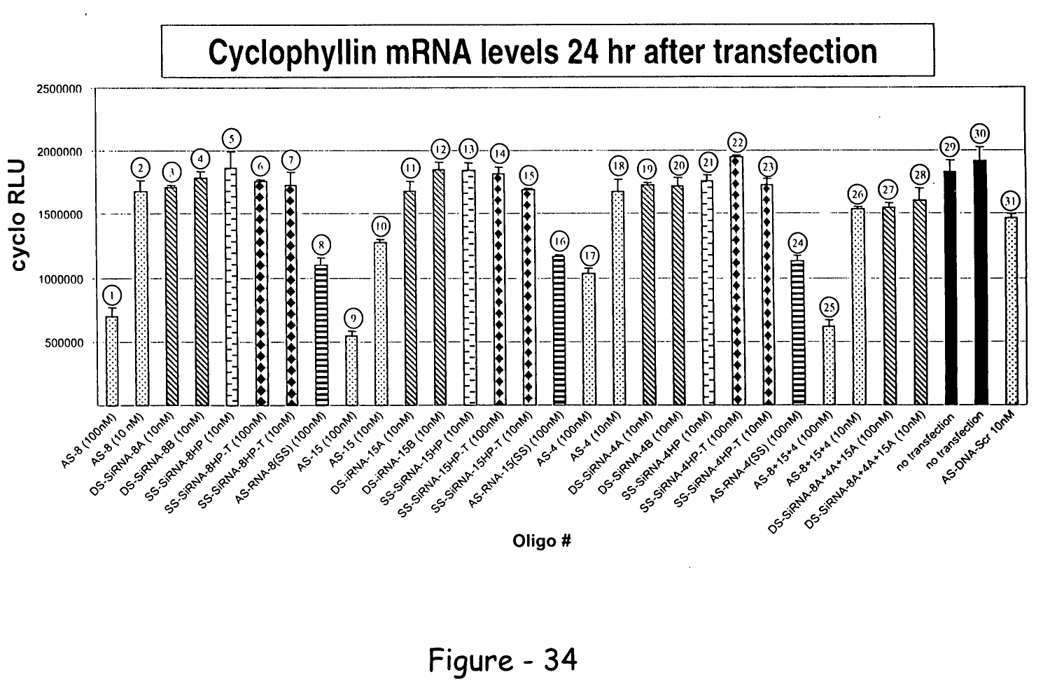

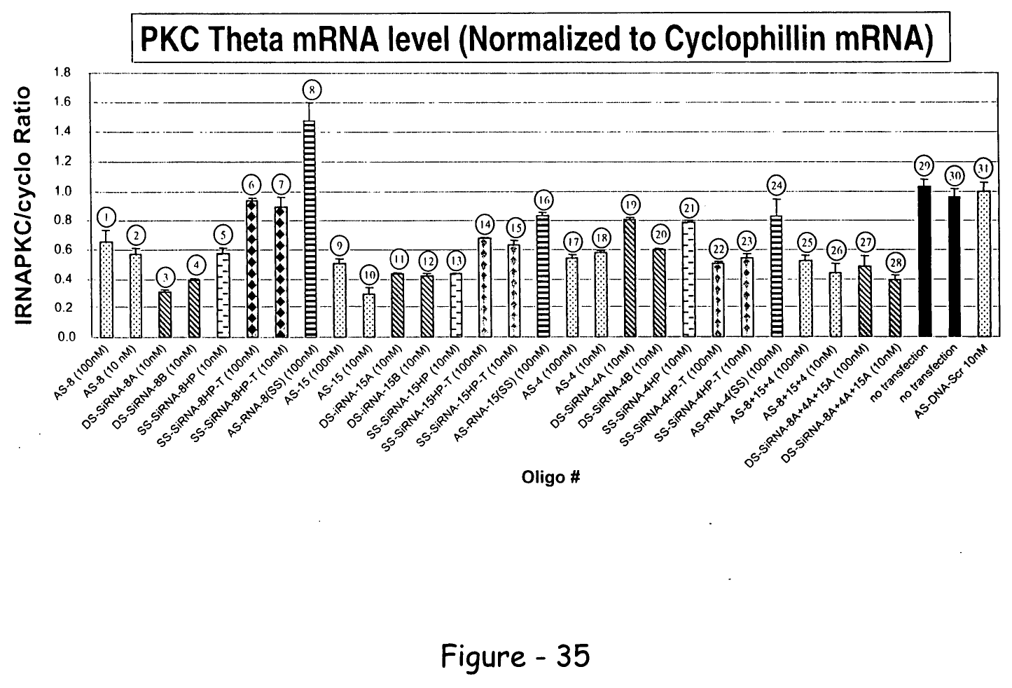

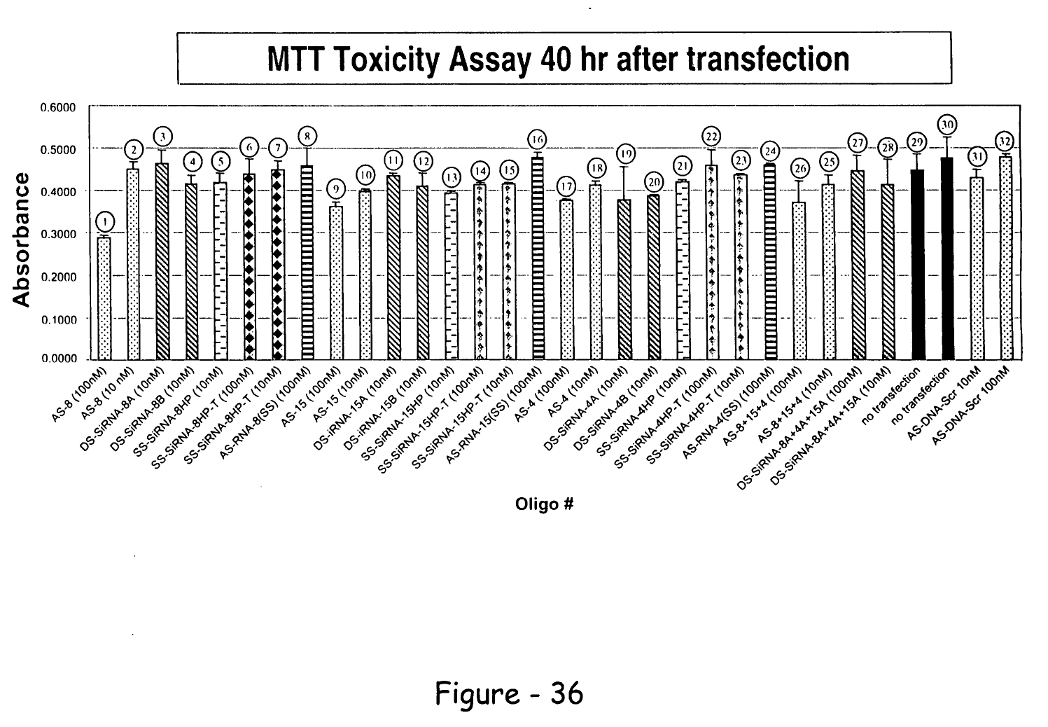

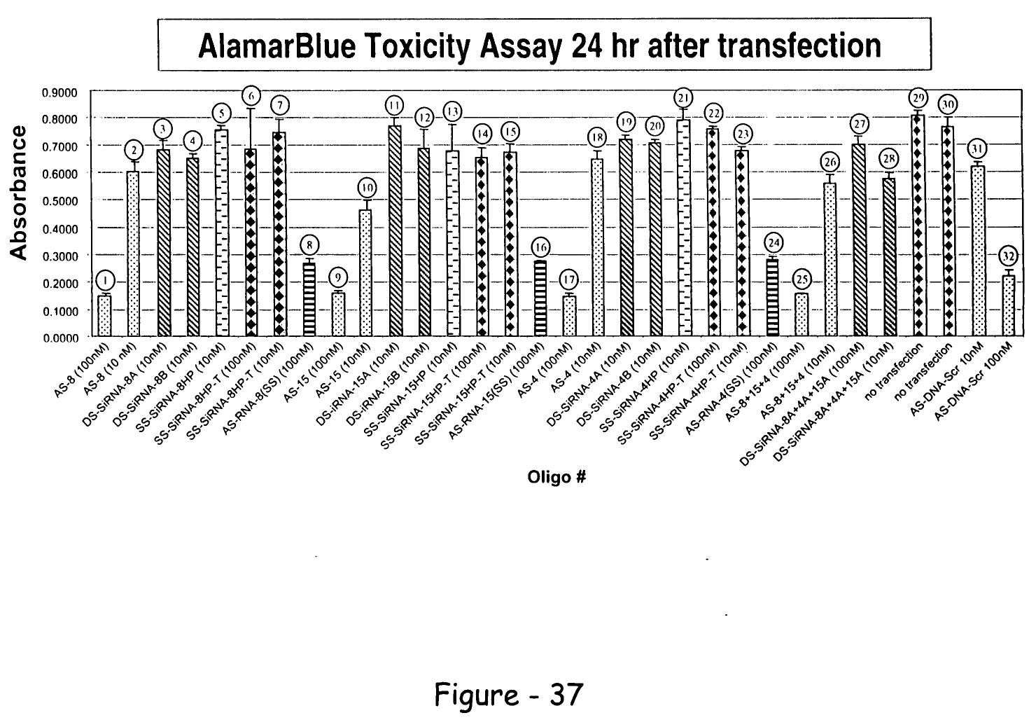

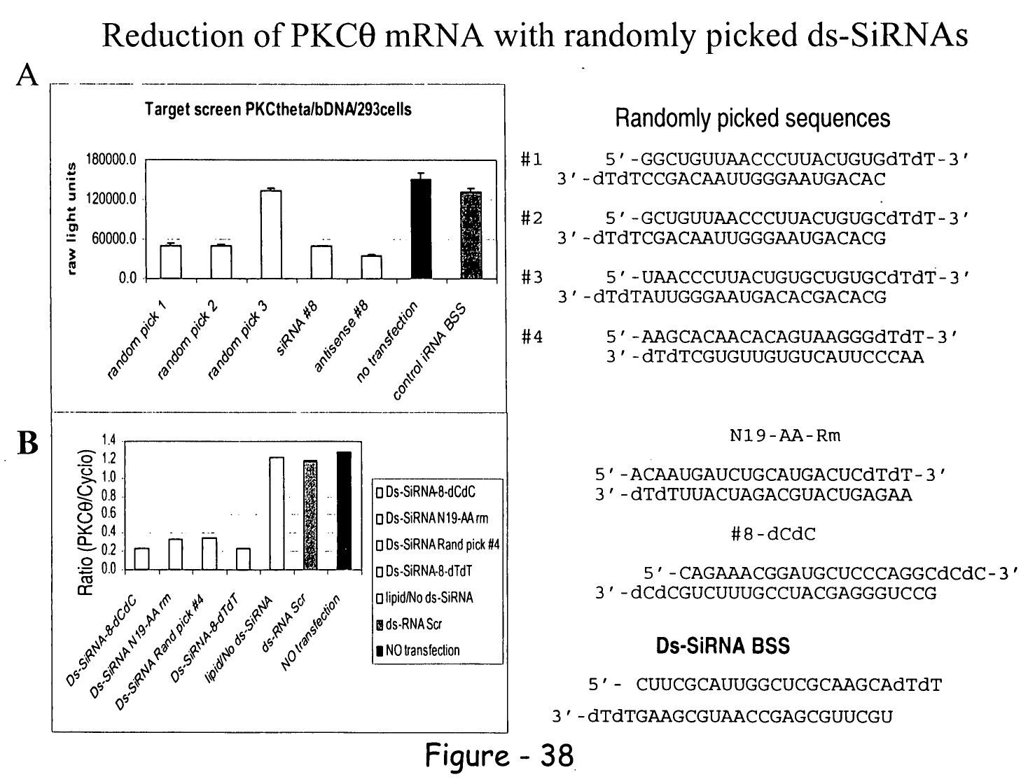

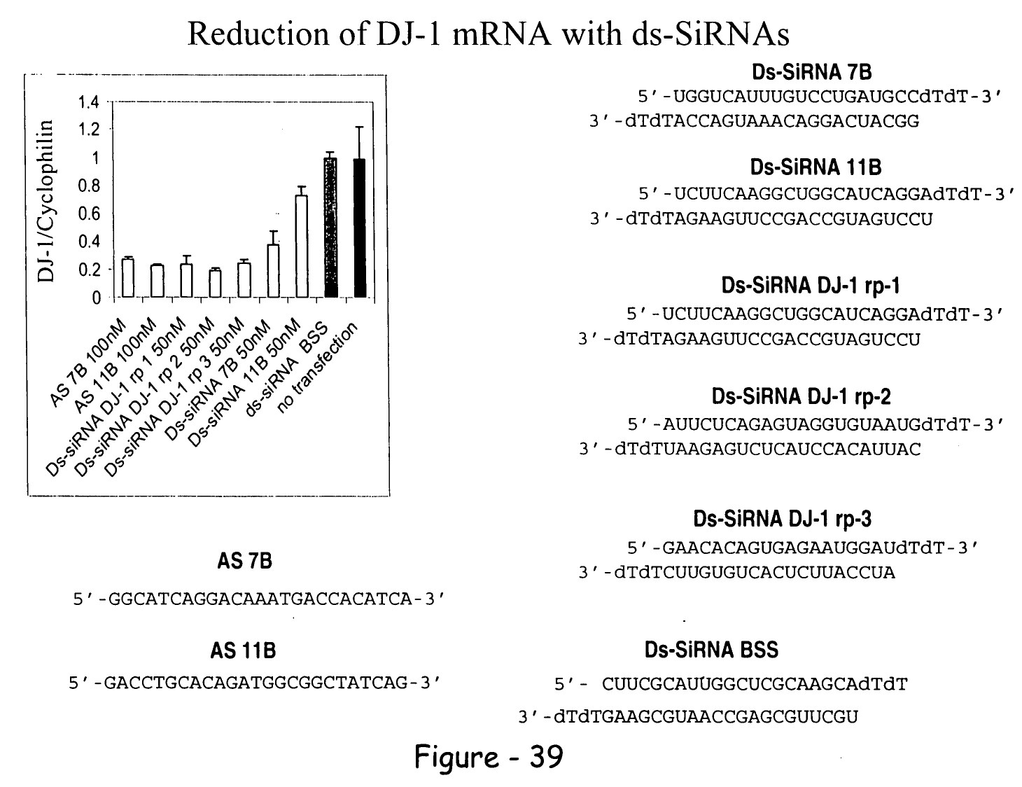

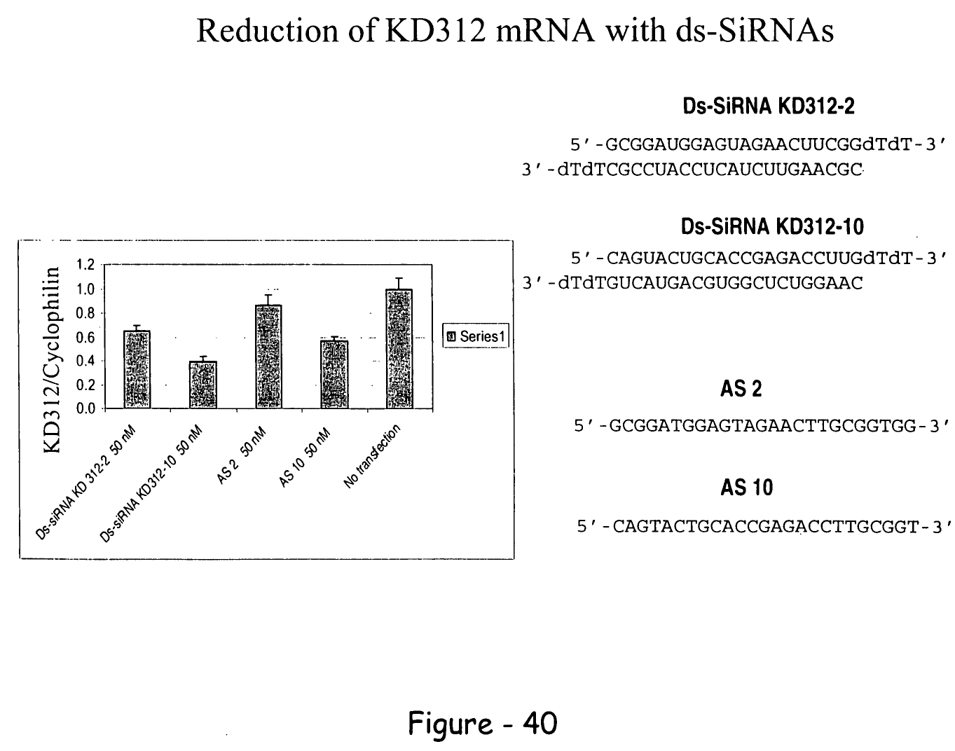

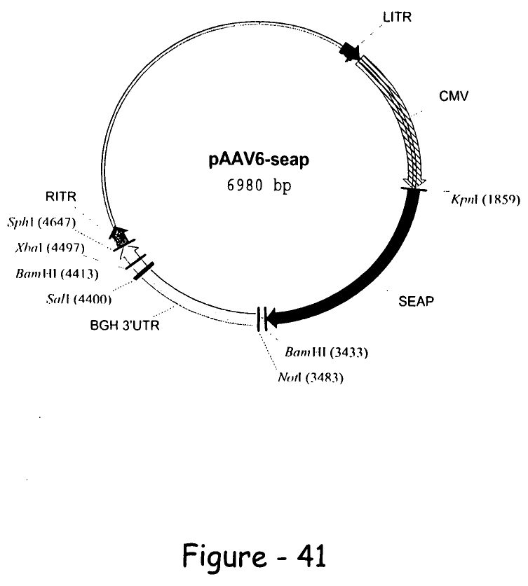

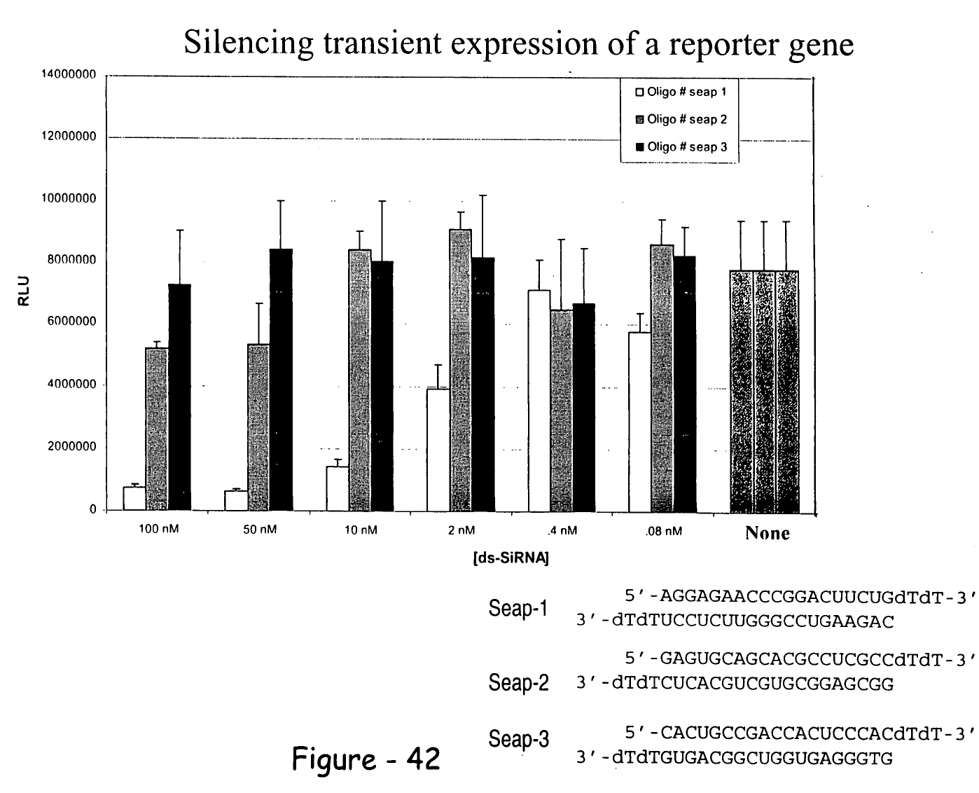

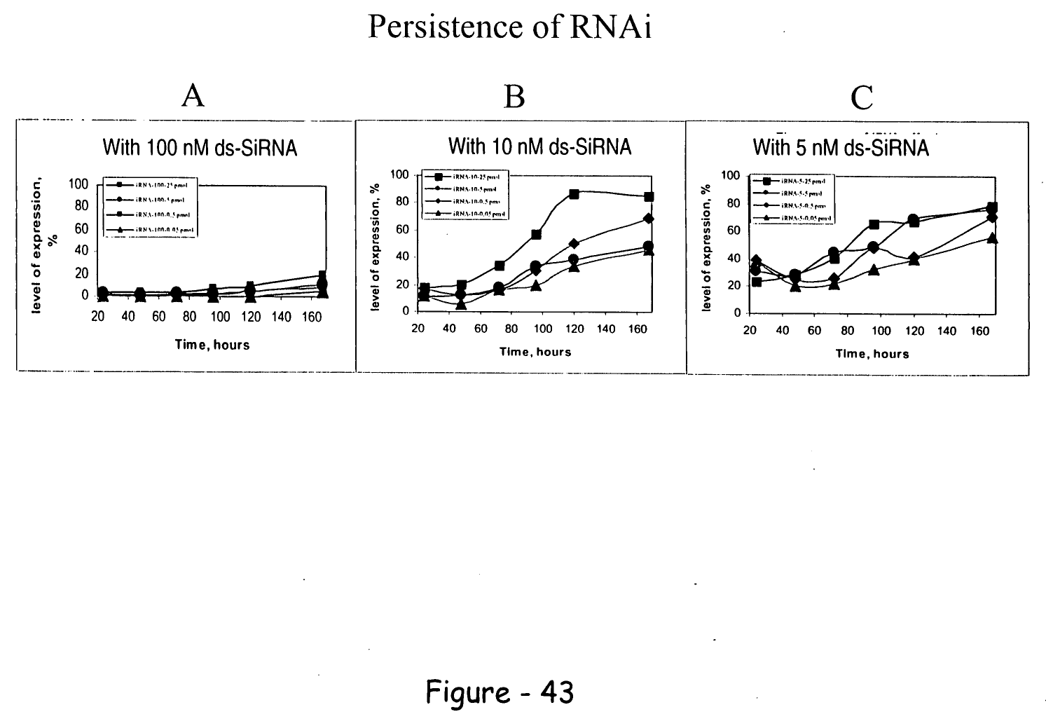

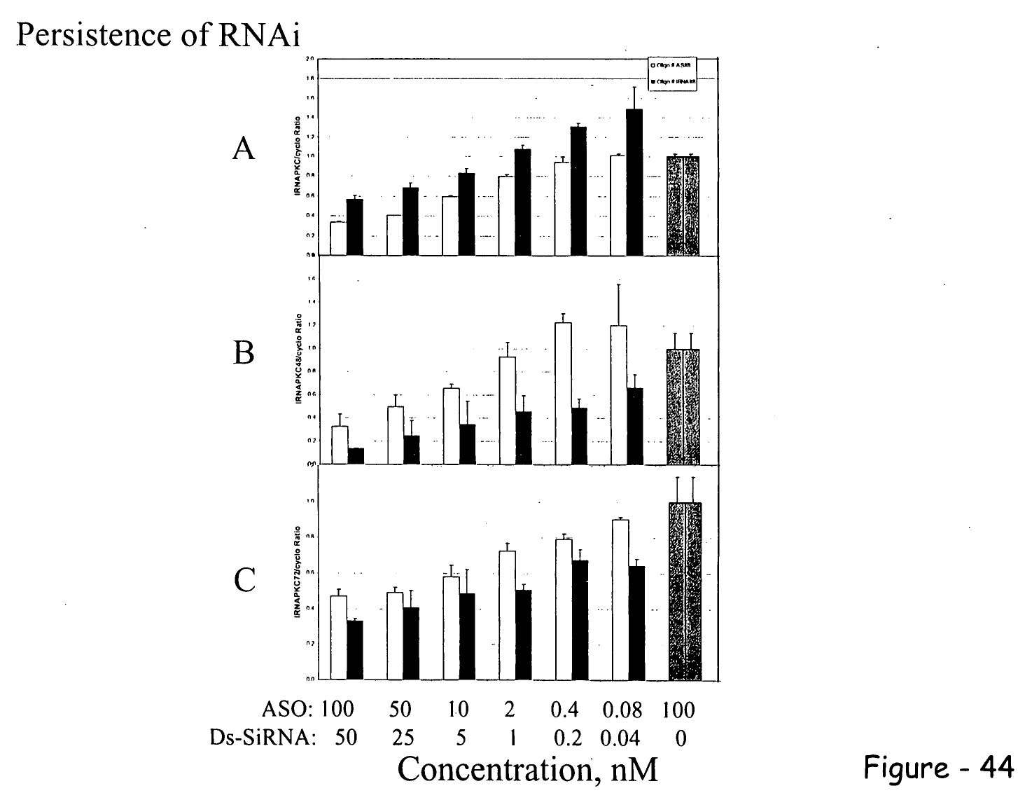

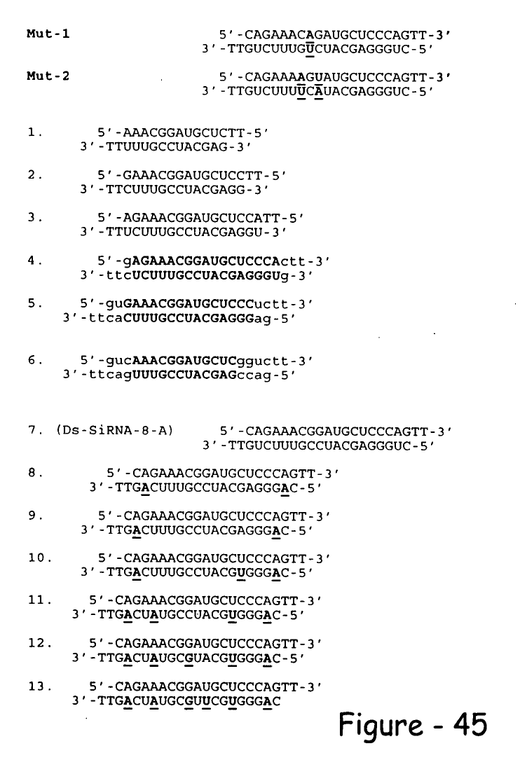

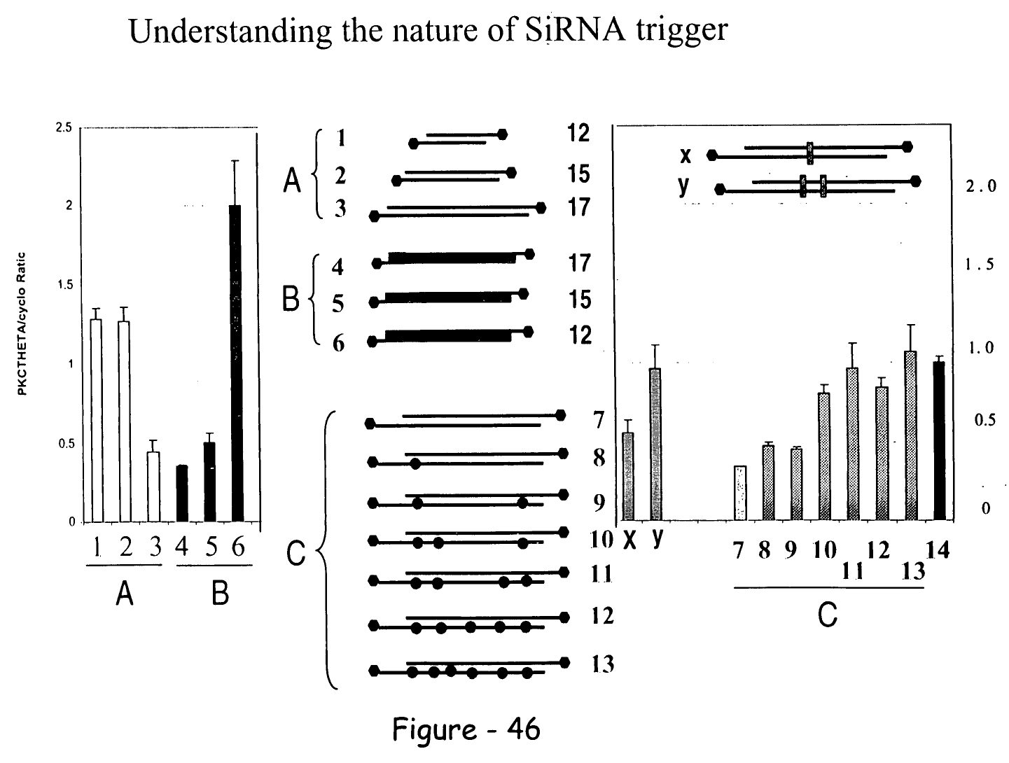

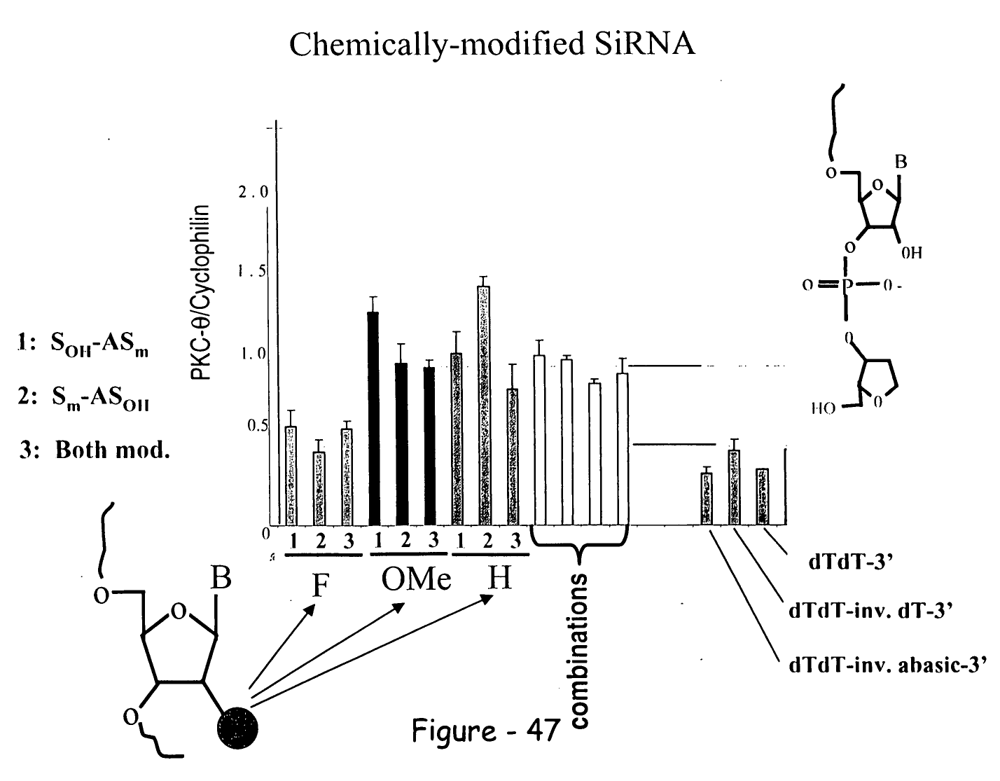

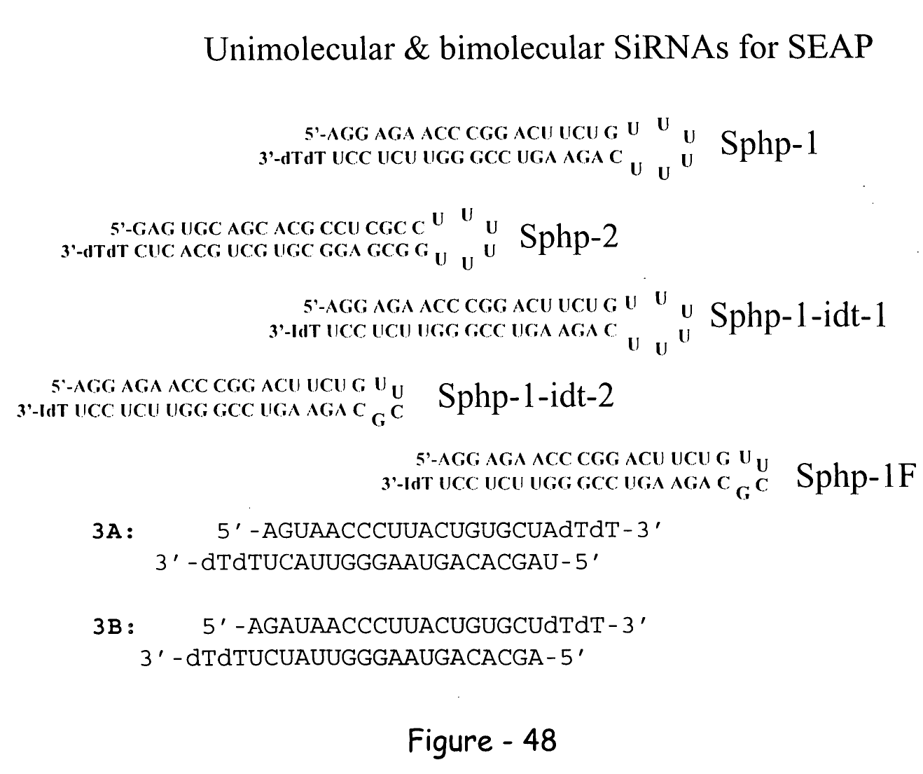

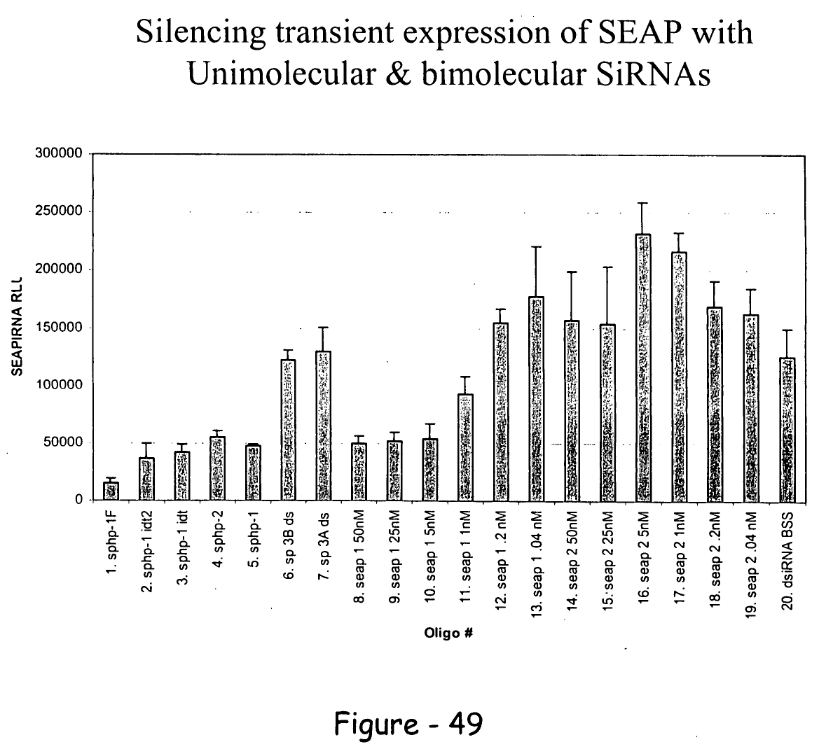

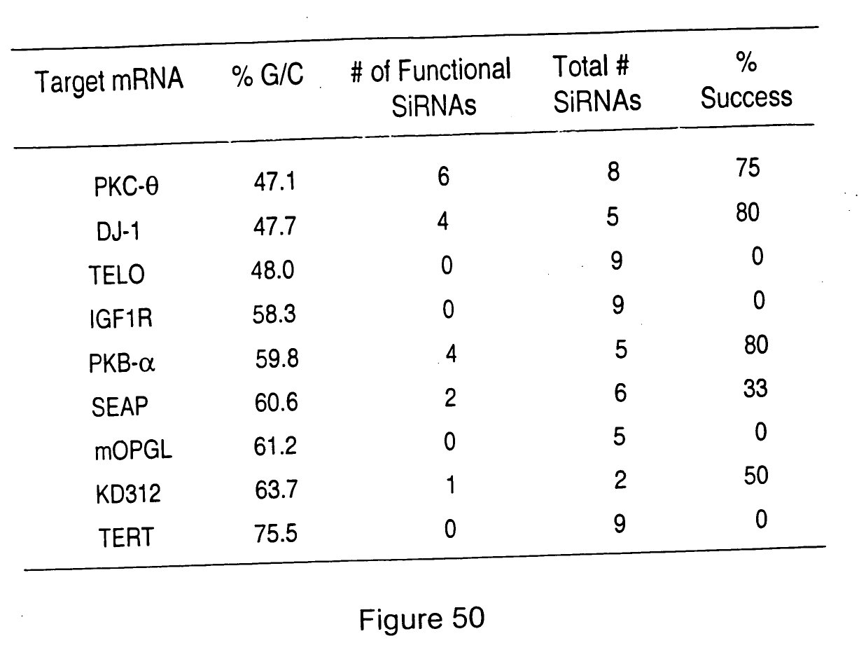

In certain embodiments, proposed strategies may be based on the use of random sequence RNA libraries to elicit an RNAi response in mammalian cells. In certain embodiments, a library of random sequence short RNA hairpins may be used to elicit RNAi-mediated gene silencing in mammalian cells. Long RNA hairpins have been used to elicit an RNAi response in several species including C. elegans (Parrish, 2000; Tavernarakis, 2000) Trypanosome (Ngo, 1998), Drosophila (Piccin, 2001) and in plants (Chuang, 2000). The use of short hairpin molecules to trigger RNAi in cultured mammalian cells was investigated. This proposition was based on preliminary data that supports successful and specific gene inactivation by short hairpin molecules with defined sequences (FIG. 6). The existing data suggests that the sequences in the loop of a short hairpin in this work do not have any effect on the efficacy and the selectivity of the RNAi process in mammalian cells. The random region may be placed within the stem of the hairpin in SIRS libraries. Different phenotypic responses (or functions) established in cells that have been exposed to short interfering random sequence (SIRS)RNA hairpin libraries may be looked for using SIRS RNA hairpin libraries. The complexity of the library may be narrowed down by serial dilution to identify small collections of hairpins that trigger the chosen cellular phenotype. Molecules within this sub-library may be split into another array to further reduce the collection of sequences of short RNA hairpins that trigger the specific phenotype. Nucleotide sequences within these hairpins may be identified by cloning and sequencing. The consensus sequence that emerges within this collection of hairpins may be subjected to a blast search to identify the potential candidate gene for the biological function.

Certain methods may be proposed to screen SIRS hairpin libraries to identify ss-SiRNA hairpins that elicit specific phenotypic responses in cells. Certain methods use a split screening approach; as described above, to cull the original library, whereas certain other methods utilize a biological screening approach using a retrovirus that expresses random sequence short hairpins.

Method 1:

Method 1 may be based on the use of in vitro synthesized SiRS RNA hairpin libraries from which RNAi triggers may be identified through repetitive screening of sub-libraries. These sub-libraries may be subjected to splitting and amplification to identify the sequence of interest.

A. Library Construction

A. 1. General Design of Synthetic DNA Templates

Short interfering random sequence (SiRS)RNA hairpin libraries may be generated from synthetic DNA templates. The design of the synthetic DNA template is illustrated in FIG. 7 (boxed). These synthetic DNA templates may be single stranded and may contain the top strand of the T7 promoter sequence at the 5′ end followed by a contiguous stretch of a random region. The promoter for RNA polymerase may be for any RNA polymerase known in the art. Certain examples of polymerases are the T7, T3, and SP6 RNA polymerases. Next to the random region may be a defined nucleotide sequence followed by a loop sequence and a sequence stretch complimentary to the defined nucleotides. In this design, the 3′ end may fold back to form an incomplete stem-loop. The. 5′ end of the DNA template may be biotinylated to facilitate its immobilization on a microtiter plate well surface to facilitate subsequent steps in the screening process. Biotinylation of the DNA template and the use of streptavidin-coated microtiter plates are not required for the screening process.

A. 2. Sequence Complexity

The number of nucleotides in the random region dictates the sequence complexity of a library. A library with 15 randomized nucleotides has 1.0×10⁹ theoretically possible individual molecules, whereas a library with 20 such nucleotides will have 1.0×10¹² individual molecules. One would expect a single 15-nucleotide-long sequence within a library of approximately 10⁹ individual molecules to occur only one time in the human genome. Based on this calculation, such a library may be expected to provide. RNAi triggers for all possible genes in the human genome. However, a SiRS RNA hairpin library having one million unique RNAi triggers may be used. This library may be large enough to capture most genes.

A. 3. Synthesis of RNA Libraries

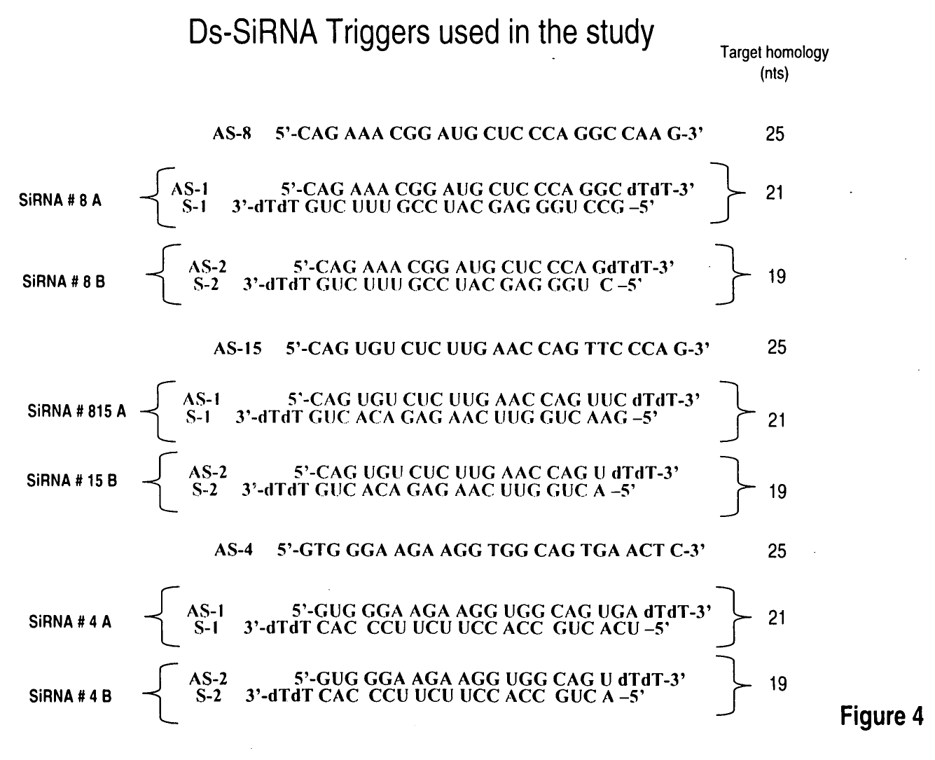

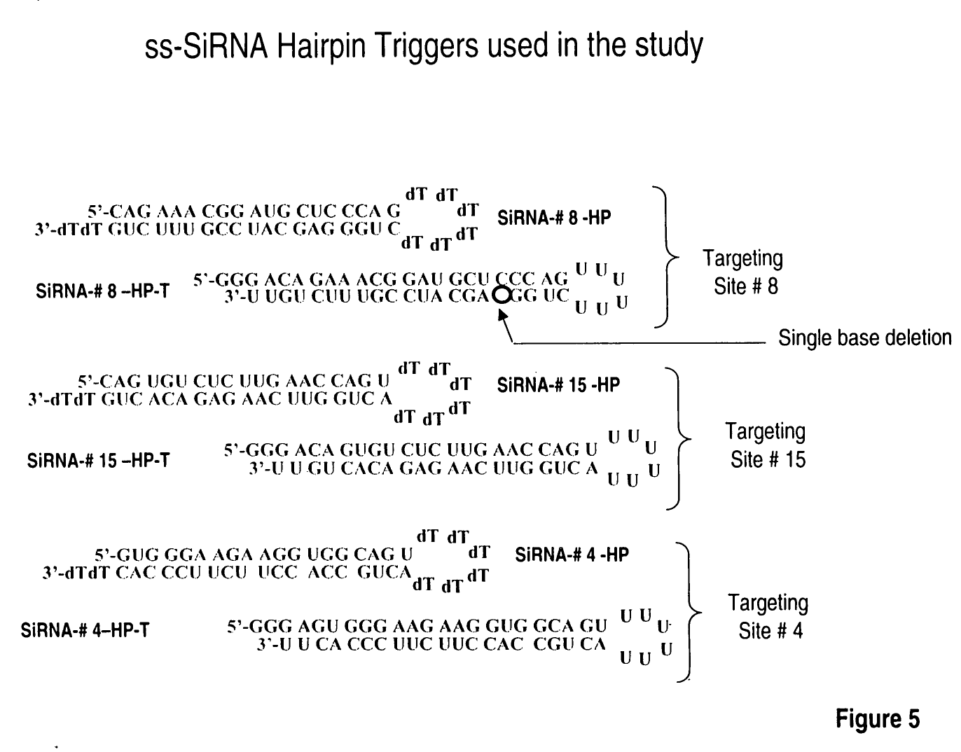

A general scheme for the production of an SiRS RNA hairpin library from the corresponding synthetic DNA template library using in vitro transcription is outlined in FIG. 4. Synthetic DNA templates may be heated to 95° C. and allowed to slowly cool to room temperature to facilitate the formation of partial stem-loop structures. The 3′ ends of partial DNA stem-loop templates may be extended by using a high-fidelity DNA polymerase such as the Klenow fragment of an E. coli DNA polymerase 1. This reaction allows for the formation of all theoretically possible combinations of perfect stems. Once the double stranded DNA template is made, it may be distributed into streptavidin-coated microliter plates (1536-well Master Plates) (FIG. 8).

Since the concentration of each template may not be sufficient to generate enough RNA hairpins of one type, copies of immobilized templates may be amplified by polymerase chain reaction (PCR). PCR-amplified DNA templates may be used for in vitro transcription using T7 RNA polymerase (when T7 RNA polymerase is included in the DNA template). The resulting single stranded RNA molecules may fold into intra-molecular hairpin structures. They also may undergo intermolecular hybridization and generate double-stranded RNA molecules. Both RNA hairpins and dsRNAs may generate an RNAi response. Thus, the formation of intra- or inter-molecular structures should not pose a problem in this strategy. Once RNA molecules are made, they may be ready for use in cell-based screening assays.

B. Functional Screening

Certain screening exercises may start with a dsDNA template library having approximately 1×10⁶ molecules that has been distributed into five 1536-well microtiter “Master Plates” resulting in approximately 130 unique templates in a single well. Once biotin-conjugated DNA templates are immobilized in the streptavidin (SA) coated “master plates”, excess SA in the well may be blocked with free biotin, and the wells may be thoroughly washed. Subsequently, each template molecule immobilized on the “master plate” may be amplified by PCR using specific primers one of which may be biotinylated at the 5′-end. Upon PCR amplification, amplified, biotinylated, template molecules may be transferred to four 384-well “lead plates” from a single “master plate” (FIG. 9). Since the “lead plates” may also be SA-coated, amplified template molecules may be captured onto the well surface. Next, SiRS RNA hairpin sub-libraries may be generated within the wells of “lead plates” using the standard in vitro transcription protocol. These SiRS RNA hairpin sub-libraries may be delivered by a standard cell transfection protocol into cells grown in 384-well plates in a geometrically addressable manner. Several different functional assays in parallel may be performed using SiRS RNA hairpin sub-libraries from “lead plates”. Hence, it may be possible to use a relatively low number of cells per well (approximately 5000) for transfecting SiRS RNA hairpin libraries. Functional screening may be based on several biological end-points. Some nonlimiting examples are listed below.

1. Cell proliferation.

2. Cell death (with and without apoptosis).

3. Cell migration.

4. Cell senescence.

5. Specific reporter assays.

6. Triggering the production (or inhibition) of secretary molecules.

Certain high-throughput cell-based assays may be used to screen an SiRS hairpin library. Each functional screening assay may give rise to the identification of one or more wells with biological endpoints of interest. Corresponding wells in the lead plate contain “lead SiRS libraries”. Since functional screening for various biological end-points using different assay formats and cell types may be carried out in parallel, several “lead SiRS libraries” may be identified simultaneously from a single lead plate. These lead libraries may be split into sub-libraries and the resulting sub-libraries may be used for further screening as described below.

Once a lead library for a particular functional assay is identified, the specific RNAi trigger hairpin may be delineated by further fractionation of the members within the library. This may be done by subjecting the corresponding DNA library in a “lead plate” to low-level PCR amplification, followed by distributing the amplified products into several 96-well “daughter plates” (FIG. 10). If one uses three 96-well plates, it may be that a very low number of unique template molecules per well (approximately 1-5 unique template/well) may be possible. A low ratio of unique templates per well may also enhance the probability of identifying the unique RNAi trigger for the specific biological outcome. After distribution, template molecules in each well of 96-well plates may be PCR amplified to enrich the number of copies of unique template molecules. RNA may be made within each well of “daughter plates” and used for secondary screening using the same functional assay as used to obtain the lead. Wells from which RNAi triggers were derived to elicit the biological end-point may be identified. If necessary, additional rounds of screening may be carried out to further reduce the complexity of sub-libraries derived from lead libraries. DNA templates from the corresponding wells in “daughter plates” may be cloned and sequenced. Sequences derived from each well may be compared to identify the consensus sequence of the RNAi trigger responsible for the observed biological end-point (FIG. 11).

The biological end-point in the particular functional screen may be confirmed by synthesizing the specific RNA hairpin molecule derived from the consensus sequence. Once confirmed, nucleotide sequences in both arms of the specific hairpin may be used to perform a BLAST search against the human genome database to identify the candidate gene (FIG. 12).

The success of the screening process may depend on the efficiency of library transfection and the amount of RNAi trigger molecules that silence a gene within a single cell. Approaches that make SiRS RNA hairpin libraries more nuclease resistant may help improve the survival of individual molecules during transfection, which may be important during the primary screening step in which the libraries are more diverse. Previous studies in C. elegans using long dsRNA triggers suggested that the substitution of 2′-F uracils in place of 2′-OH uracil in either sense or antisense strarid did not interfere with the RNAi process (Parrish, 2000). These authors also reported successful RNA interference with long dsRNA triggers synthesized with a single type of α-thio NTP. Substitution of α-thio UTP may produce a somewhat reduced level of RNA interference. A successful reduction of specific mRNA in mammalian cells was observed using SiRNA triggers of 21 nucleotides in which all pyrimidines in both strands are modified with 2′-F sugars. The incorporation of 2′-F-modified pyrimidines and α-thio purines into hairpins may make them nuclease stable. SiRS RNA hairpin libraries with chemically-modified pyrimidines on the sugar and phosphorothioate backbone modifications at all purines may be obtained by in vitro transcription employing the appropriately modified NTPs.

Another approach to enrich the population of each hairpin molecule within target cells employs the transfection of ds-DNA templates (preferably linear or circular in the form of plasmids) carrying the T7 promoter fused to the template for hairpin synthesis. In this case, the target cells may carry the T7 RNA polymerase gene integrated into their genome. Upon transfection, T7 RNA polymerase will transcribe many copies from each template within the cell.

Method 2:

Method 2 uses a biological screening method using a retroviral vector carrying SiRS RNA hairpin libraries. Retroviruses that mediate a specific biological function in infected cells may be identified, amplified and used for the next cycle of selection.

A retroviral screening system employing random sequence combinatorial libraries containing the target recognition site for a hairpin ribozyme has been used for target validation. (Kruger, 2000; Li, 2000)

A. Library Construction

DNA constructs that express SiRS hairpin RNA libraries in vivo may be designed as illustrated in FIG. 13. A synthetic single-stranded DNA molecule in which two defined regions flank a randomized region may be obtained by chemical synthesis. The ss-DNA template may be converted to ds-DNA by PCR amplification using a primer pair that anneals to the two defined regions. The two PCR primers may carry two unique restriction sites near their 5′ ends. As shown in FIG. 13, the dsDNA may be digested with one restriction enzyme and the products may be gel-purified. The resulting cohesive ends may be ligated to obtain dsDNA carrying the inverted repeat. These inverted repeats may be cloned into a retroviral vector after digestion with the second restriction enzyme using standard cloning techniques known in the art. For efficient synthesis of SiRS RNA hairpins, the dsDNA library may be cloned downstream of the human pol III tRNA (e.g., tRNA val) promoter which is known to provide a high level of transcription in vivo (Good, 1997). Antibiotic markers such as neomycin and puromycin may be inserted into the retroviral vector to facilitate the selection of vector containing cells after transduction. Several picomoles of the dsDNA library, representing approximately 10¹¹ individual molecules, may be ligated into the retroviral vector. The entire ligated mixture may be transformed into a bacterial strain and plated on approximately 10 plates. Transformants may be identified, pooled, and stored at −80° C.

Plasmid libraries may be obtained by growing the transformant pool followed by carrying out minipreps. The plasmid DNA library may be used to generate the retroviral vector library by triple transfection methods known in the art. This may include the cotransfection of the plasmid DNA library along with two vectors, one expressing Gag-Pol (Landau, 1992) and the other expressing VSG-G (Burns, 1993) into a packaging cell line. The supernatant containing the retroviral library may be harvested, filtered, and used for biological screening.

Biological screening, as in Method 1, may be performedin parallel. In each case, cells may be transduced with SiRS RNA hairpins carrying retroviral vector at a very low MOI (multiplicity of infection), in certain instances, as low as 1. Cells may be maintained in the presence of the antibiotic to which the retrovirus is resistant and screened for a desired biological response. Individual wells exhibiting a desired biological outcome may be identified and viruses may be rescued from them.

Viral rescue from selected wells may be achieved by co-transfecting DNAs of two helper vectors expressing Gag-Pol and VSV-G. After co-transfection, the vector supernatant may be selected, pooled, filtered, and used for the next round of selection with a fresh plate of cells. Alternatively, PCR rescue may be performed to rescue the sub-library of inserts that provided the desired biological outcome. A single PCR primer carrying the second restriction site in the original DNA construct may be used for PCR rescue. For this, high molecular weight DNA may be extracted from identified wells and PCR amplified with the primer. Resulting PCR products may be digested with the second restriction enzyme, gel purified, and cloned into the retroviral vector as described above. The resulting retroviral sub-library may be used for the next selection round.

After 2-3 cycles of selection-enrichment of retroviral libraries, inserts may be cloned and sequenced to identify a consensus sequence motif. Based on the consensus sequence motif that emerges, a hairpin RNAi trigger may be synthesized and used to confirm the biological effect of the trigger. Once confirmed, nucleotide sequences in both stems of the RNAi trigger may be used to perform a blast search against the human genome database to identify the candidate gene.

III. Certain Methods for Designing Functional SiRNA Triggers for Effective Gene Silencing by RNA Interference

RNA interference (RNAi) is an effective technique for gene silencing in many organisms, including mammals. Short RNA duplexes of 21-22 nucleotides with 2-3 nucleotide 3′ extensions, generally referred to as SiRNA molecules, have been effective in specific gene silencing in mammalian cells by triggering the RNAi process. However, not all SiRNA molecules bearing homology to a region within an mRNA sequence work effectively in silencing the cognate gene. A simple, effective, and universal approach is described here for designing productive SiRNA triggers using at least one of the following two criteria: (1) a relatively low calculated melting temperature in the range of 55°-70° C.; and (2) a calculated low energy (−6 to −9 kcal/mol) internal stability profile with either a flat profile or a bell-shaped distribution-suggesting a high internal stability concentrated in the middle of the duplex. Analysis of more than 35 SiRNA triggers targeted to several human genes revealed that triggers that satisfy these two criteria were generally effective in silencing the targeted gene. The proposed approach has been experimentally validated by designing functional SiRNA triggers according to the criteria outlined in the method. Hence, choosing SiRNA triggers guided by the proposed approach may be helpful in avoiding the synthesis of a large number of triggers for a given target. Therefore, this technique may represent a more economical and efficient way to analyze gene function.

A. Introduction

Specific gene silencing has a wide range of applications in biology. Some of these applications include the understanding of the function of a gene, elucidating the individual role of a gene in a complex biological pathway, and the identification of novel therapeutic targets. Validation of therapeutically relevant gene targets may be valuable for the pharmaceutical industry in general, and may also have a broader societal impact in improving the quality of human life. In this regard, techniques that allow for specific gene silencing may play a role in achieving these goals. Some have used the antisense approach at the tissue culture level for target validation. An approach called RNA interference (RNAi) has emerged as an effective way to achieve specific gene silencing. In certain instances, RNAi is a post-transcriptional phenomenon mediated by double-stranded RNA (ds-RNA) with one strand bearing homology to the mRNA of the gene to be silenced. RNAi has been described in the nematode Caenorhabditis elegans (C. elegans) using long dsRNA molecules (Fire, 1998; Guo, 1995). RNAi also has been demonstrated in a wide range of species (reviewed in Bosher, 2000; Hammond, 2001; Sharp, 2001; Zamore, 2001). Although the exact mechanism of RNAi is not completely understood, an in vitro system derived from Drosophila embryonic cells that recapitulates the silencing event has provided some mechanistic insights into the process (Hammond, 2000; Tuschl et al., 1999). These studies led to the demonstration that the long dsRNA molecules that activate the RNAi process are cleaved into fragments of approximately two helical turns (approximately 21′-22 nucleotides) (Zamore, 2000) by a multidomain ribonuclease III protein called Dicer (Bernstein, 2001). These short dsRNA may then become a platform on to which an ensemble of proteins assembles to form an RNA induced silencing complex (RISC). While the antisense strand of RISC may guide substrate recognition, an endonuclease may perform the cleavage of the target RNA (Elbashir, 2001). The short dsRNA molecules, derived from a long ds-RNA sequence, that become a part of the RISC are called short interfering RNA molecules (SiRNAs) (Elbashir, 2001).

While certain long dsRNA molecules homologous to a target mRNA may work effectively in lower organisms, in certain instances, they pose a challenge in mammalian cells. In response to the exposure to long dsRNA, mammalian cells are known to activate cellular pathways that lead to the global shut down of gene expression. This nonspecific inhibition of gene expression may be a result of an antiviral response mediated by interferon gamma and RNA-dependent protein kinase pathways (Geiss, 2001; Stark, 1998). Consequently, in certain instances, the use of long dsRNA in mammalian cells for RNAi-mediated gene silencing has been unproductive. However, recent experiments in the field suggest the use of SiRNA molecules as an effective trigger for silencing genes in mammalian cells. In contrast to long dsRNA molecules, the short length of SiRNA may bypass the antiviral response. This discovery opened up the use of an RNAi approach for analyzing mammalian genes, including those of humans. SiRNAs have been designed to carry 3′ extensioris with two nucleotides that mimic the products of Dicer cleavage. To confer protection from potential 3′-5′ exonucleases, the two nucleotides in the 3′ extensions may be substituted with dT, instead of natural RNA bases.

A procedure to design SiRNA molecules with dT 3′ extensions has been suggested by Tuschl and colleagues ((http://www.mpibpc.gwdg.de/abteilungen/100/105 SiRNA.html) and (http://www.dharmacon.com/tech/tech003.html). This suggested approach for choosing SiRNAs involves the following criteria: (1) locate the first AA dimer 75 nucleotides downstream of the start codon within the mRNA of the gene; (2) record the next 19 nucleotides following the AA dimmer; (3) calculate the GC (guanosine and cytidine) content of the 21 nucleotide sequence to see if it is within 30-70%; (4) perform a BLAST search against the EST database with the 21 nucleotide sequence to make sure only the gene of interest is targeted.

In order to silence a gene of interest using RNAi, it is customary to synthesize a handful of SiRNA triggers that target 19-22 nucleotide regions in the target mRNA. Out of this collection of molecules, one SiRNA molecule may be effective. This has been the case for some genes, but not for every gene. Additionally, for some targets, there are instances where none of these molecules in the first round of screening will work in triggering the RNAi process. If this occurs, the researcher must screen another set of SiRNAs with the hope of identifying one that will work. This can become an expensive exercise, especially given that the cost of RNA synthesis can be ten times as high as that for DNA. This scenario still occurs whether one arbitrarily screens SiRNA molecules or designs them using the approach described by Tuschl and colleagues (http://www.mpibpc.gwdg.de/abteilungen/100/105/SiRNA.html). Hence, criteria to intelligently guide researchers through the process of designing effective SiRNA molecules may minimize the overall cost of target validation by effectively reducing the number of SiRNA molecules required for screening per target, as well as reducing the associated burden of running additional functional assays.

While SiRNA molecules may regulate specific gene expression through targeted mRNA degradation, small temporal RNAs (StRNA) may regulate developmental timing by causing sequence-specific repression of mRNA translation. Similar to SiRNA, StRNA appear to be excised from long RNA molecules by the Dicer ribonuclease, and hence may be of similar size, 21-23 nts. Certain StRNAs that have been identified by genetic analysis were lin-4 and let-7 in C. elegans (Hutvagner et al., 2001.; Rasquinelli, 2000). During the search for SiRNAs in Drosophila embryonic cell extract, Tuschl and colleagues identified several other small RNAs of the same size (Largos-Quintana et al., 2001). This novel class of RNA, now referred to as microRNA (mRNA), is evolutionary conserved among invertebrates and vertebrates. Initial experiments suggest these mRNAs may serve as gene regulators during development mRNAs have been identified in C. elegans, Drosophila melanogaster embryonic extracts and cultured human cells (Largos-Quintana et al., 2001; Lau et al., 2001; Lee and Ambros, 2001).

These mRNAs served as a rich source of sequence information to look for cues to design effective SiRNA molecules. Certain criteria present in mRNA molecules were identified. A collection of arbitrarily designed and functionally validated SiRNA molecules were also analyzed. This analysis revealed that, in general, these criteria were met in certain SiRNA molecules that function in gene silencing, but were not found within certain nonfunctional SiRNA molecules. This result suggests that these characteristics may be important for functional SiRNAs. Certain functional SiRNA triggers were designed according to the guidelines that were identified. Analysis of these designed SiRNA triggers suggested that they are functional in RNA interference in mammalian cells. Therefore, rationally designed SiRNA triggers may not only be effective in eliciting RNAi response, but may also work at very low concentrations. These features may be helpful in therapeutic applications of SiRNA triggers by lowering the effective dose and improving the efficacy of the procedure.

B. Results and Discussion

The observed failure to silence a gene by certain SiRNA molecules bearing homology to an arbitrary chosen site within the cognate mRNA invokes certain non-exclusive possibilities. First, the possible inability of the SiRNA within the RISC to access the target site within the substrate mRNA may be responsible for this observation. Either the folding of mRNA to conceal the target site or the occupancy of the site by RNA binding proteins may dictate the target site accessibility. Unlike the RNAi process that is facilitated by a number of proteins, the antisense process relies on the passive hybridization of antisense oligonucleotide strands to the target mRNA. For several mRNAs, certain SiRNA molecules were designed to target the same sites that antisense molecules have successfully targeted, and no correlation between antisense effect and RNAi was observed (unpublished observation). These observations suggested that target site accessibility alone is not necessarily enough for SiRNA molecules to initiate RNAi. Furthermore, addressing the issue of target site accessibility may be complicated and rather complex, primarily due to the fact that the RNA folding pattern will vary from target to target. As a result, it may be optimal for interfering RNAs to be tailor made to target individual mRNAs.

Another possibility for the inefficiency of certain SiRNA molecules to trigger RNAi could lie in the innate characteristics of the targeting SiRNA. Consequently, identification and understanding of the characteristics of SiRNA molecules that favor the RNAi process may be useful.

Analysis of a naturally occurring population of RNA molecules that may be processed by a mechanism similar to RNAi may yield some insight into the RNAi process. To that end, mRNA sequences were chosen that were identified recently in three organisms, C. elegans (Lau et al., 2001; Lee and Ambros, 2001), D. melanogaster and H. sapiens (Largos-Quintana et al., 2001). These mRNAs, embedded within long precursor RNA molecules as stem-loop structures, are processed by Dicer. The stems within which mRNAs exist before processing contain bulges and G/U wobble pairs. Hence, mRNAs depart structurally from certain SiRNAs that typically contain perfect helices. Furthermore, there may be differences between mRNAs and SiRNAs on the basis of Dicer processing. In the case of mRNAs, RNA from only one arm of the stem-loop precursor accumulates in the cell (Hutvagner et al., 2001), whereas both antisense and sense strands exist in SiRNA molecules. The nature of the two nucleotides in the 3′-extension (usually dTdT) may not affect the efficacy of RNAi (unpublished observation). So, SiRNA molecules may be treated as duplexes of 19 base pairs. To be consistent with this, each mRNA may also be calculated as 19 nucleotides in length. One may also use sense strands with perfect base pairing for mRNA duplexes, which may be different from their natural form.

The average internal stability of the mRNA duplexes of the three organisms was investigated (FIG. 16, A). Since an internal stability of a base pair within a duplex may be calculated using the contribution of its nearest neighbor, the two terminal base pairs were omitted to avoid the end effects. In all cases, the average internal stability of duplex mRNAs may be in the range of approximately −6.5 to −8.0 Kcal/mol. The distribution of the average internal stability of the internal 17 nucleotides forms a curve somewhat resembling a bell shape with high internal stability concentrated in the middle nucleotides compared to the two ends. This implies that the middle part of the mRNA duplex may be relatively stable while the two ends may fray easily. This profile was very distinct in C. elegans sequences in category I (Lee and Ambros, 2001) and D. melanogaster sequences in category II (Largos-Quintana et al., 2001) compared to those in the other two categories. MiRNA sequences in categories III and IV have relatively stable nucleotide pairs near the 5′ end of sequences. To investigate whether a collection of any 19 nucleotide RNA duplexes, chosen randomly, would also have a bell-shaped average internal stability profile, mRNA sequences of three different genes were chosen, and the average internal stabilities of duplexes resulting from 19 nucleotide antisense strands annealed from one end to the other (FIG. 16, B) was calculated. The calculated average internal stabilities of all three random collections of 19 nucleotide RNA duplexes, derived from three genes, were higher than those of the four mRNA duplex populations. Furthermore, their average internal stability profiles do not have the bell-shaped appearance. This may suggest that the calculated average internal stability profile, characteristic to mRNA duplexes, may be a special feature to RNA duplexes processed by Dicer and utilized by cellular mechanisms analogous to RNA interference.

The average internal stability of a nucleic acid duplex also reflects its propensity for melting. The calculated average melting temperatures (Tₘ values) of mRNA duplexes are relatively low, and hence agree with their low average internal stabilities. The average Tₘ values within the four groups of mRNA duplexes vary from 52°-58° C. (FIG. 16, C). In contrast, the average Tₘ values of the collections of 19 nucleotide duplexes, scanning the entire mRNAs of the three genes, are higher (60°-66° C.) than those of mRNA duplexes. In fact, the calculated Tₘ values and internal energy of mRNA duplexes may be higher than in natural forms containing unpaired nucleotides and G-U wobble base pairs. In reality, these values may be even lower than the values presented here. It is noted, however, that the average Tₘ of 19 nucleotide RNA duplexes may vary depending on the G/C content of the genes that are chosen. Since Tₘ values of nucleic acid duplexes of the same length may vary as a function of their G/C content, the G/C content of mRNAs was also analyzed. The G/C content of antisense strands of mRNAs was below 50% (40-44%), whereas those of the random collections of 19 nucleotide RNA duplexes were between 48-60%.

MiRNA duplexes may be distinguished from the random RNA duplexes based on certain parameters; a bell-shaped calculated average internal stability profile and a low calculated Tₘ value. A collection of SiRNA molecules were analyzed. This collection included a series of SiRNA molecules that were experimentally validated against six different human genes, one mouse gene, and a single reporter gene SEAP (secreted alkaline phosphatase). In this collection, a total of 37 SiRNA molecules were tested, out of which 16 were effective in reducing the level of a targeted mRNA by greater than 70%. These molecules were mostly designed arbitrarily, although some were picked by using the suggestions made by Tuschl and colleagues (http://www.mpibpc.gwdg.de/abteilungen/f100/105/SiRNA.html). The average internal stability profiles of the two classes within the collection of experimentally validated SiRNA molecules were analyzed (FIG. 17A). The average internal stability profile of the functional SiRNAs (closed squares, FIG. 17A) exhibits a bell-shaped curve with an overall low internal energy (−6 to −8 kcal/mol), a profile reminiscent of the profiles of the miRNA duplexes. On the other hand, the collection of nonfunctional SiRNAs exhibits a markedly different average internal stability profile that may be characterized by overall high internal stability (−8 to −15 kcal/mol) and a lack of (or the mirror image of) a bell-shaped curve. Thus the analysis of the internal stability profiles for the two classes of SiRNA molecules suggests that a bell-shaped internal stability profile may be associated with productive SiRNA triggers that effectively promote RNA interference.

The calculated melting temperatures of the two classes of SiRNAs (FIG. 17B) provides another parameter that may be important for defining the effectiveness of an SiRNA molecule. SiRNAs in both classes have a broad range of calculated Tₘ values. Certain functional SiRNAs have Tₘ values from 48°-70° C., with an average Tₘ of 58.2° C., which is close to the average Tₘ of the miRNA duplexes (54.5° C.). In contrast, the Tₘ values of certain nonfunctional SiRNAs ranged from 55°-83° C. with an average Tₘ of 70.9° C. In general, the latter class has a higher Tₘ value than the former, reflecting their overall high average internal stability. This analysis of Tₘ data suggests that, in general, SiRNAs with relatively low Tₘ values (close to 55° C.) may be attractive as triggers that promote the RNAi process. The average G/C content of certain functional SiRNAs is 52%, whereas that of certain nonfunctional SiRNAs is 72%, again suggesting that more stable SiRNA duplexes, in certain instances, may be generally nonfunctional.

Interestingly, there may be SiRNAs in both classes, functional and nonfunctional, with calculated Tₘ values between 55° and 70° C. The subpopulation of SiRNAs with calculated Tₘ values between 55° and 70° C. from both classes were analyzed and their average internal stability profiles were calculated (FIG. 17C). The two classes of SiRNAs have internal stability profiles that were almost mirror images. Certain functional SiRNAs exhibit an average internal stability profile with a bell-shaped distribution, the characteristic signature for productive SiRNA triggers in certain instances. Whereas, certain nonfunctional SiRNA molecules, with their melting temperatures within the same range, have an average internal stability profile reflecting the mirror image of a bell-shaped curve. This analysis suggests that, in certain instances, when one picks two SiRNAs with roughly the same Tₘ values, e.g., in the 50°-70° C. range, the one with a bell-shaped internal stability profile may be more likely to function as a productive trigger.

It may be that one will find SiRNA molecules that are either functional, but may not follow the criteria, or have the criteria fulfilled but fail to function. In fact, such outliers were searched for in the rather small collection of experimentally validated SiRNAs, and molecules in either category that failed both criteria were not found. However, two sets of examples in which SiRNAs fulfilled one criterion but not the other were found, (FIG. 18A, B and C). In the first case (Panel A), the two SiRNAs targeted to two different genes, PKC-θ and PKB-α, have internal stability profiles close to the profile preferred for a productive RNAi trigger. However, the one with a calculated Tₘ of 66° C., indicated by closed squares, was functional in silencing the cognate gene. The other SiRNA, which was nonfunctional, had a calculated Tₘ of 85° C. However, the nonfunctional SiRNA also had a bell-shaped internal stability profile. In the second case (Panel B), neither of the two SiRNAs targeted to two different genes had the preferred internal stability profile. However, the SiRNA molecule with a relatively flat internal stability profile (ΔG approximately −6 kcal/mol) and low calculated Tₘ (53° C.; closed squares) was effective in gene silencing. The one that had a somewhat bell-shaped internal stability profile was nonfunctional, perhaps due to its high calculated Tₘ value (79° C.).

An example in which three SiRNA molecules are targeted to a single gene is depicted in Panel C of FIG. 18. All three SiRNAs had overlapping sites within the targeted mRNA of the PKC-θ gene. Two SiRNAs, 1 and 2, (closed squares) had almost identical target sites, displaced by only a single nucleotide. Both these SiRNAs were functional and had the preferred internal stability profile and calculated low Tₘ values (55° and 57° C.). The third SiRNA still targeted approximately the same site but was shifted by five nucleotides. This displacement by five nucleotides resulted in a different internal stability profile, which was the mirror image of the preferred form. Interestingly, this SiRNA (open squares) may be nonfunctional, although it also has the calculated Tₘ of 55° C. This example suggests the importance in certain instances of the internal stability profile for an SiRNA molecule to be productive as an RNAi trigger when its calculated Tₘ is in the preferred range. This example suggests that picking SiRNA triggers based on the calculation of Tₘ alone, in certain instances, may not be helpful if the internal stability profile is not preferred. However, SiRNA triggers characterized by Tₘ and a low internal stability profile that is flat across the duplex may also be functional (FIG. 18B, closed squares and see below).

So far an analysis of the experimentally validated SiRNA collection with respect to certain criteria suggests a good correlation between the efficacy of SiRNA molecules and meeting the criteria. To put the proposed method of choosing functional SiRNA molecules using certain criteria to the test, six SiRNA molecules were designed to target mRNA of SEAP (FIG. 19). Out of six SiRNAs, four were chosen to be functional (SEAP-309, -1035, -1795 and -2217); three SiRNA molecules met both criteria (SEAP-309, -1035 and -1795), whereas one molecule (SEAP-2217) possessed a calculated low Tₘ value (46.4° C.) and had a relatively low and flat internal energy profile (FIG. 19A-1). This trigger was specially chosen to test whether in this instance a low internal stability energy profile with a more or less even distribution across the duplex is acceptable as a functional SiRNA molecule. Two SiRNA molecules were designed (SEAP-1070 and -1260) that did not meet the two criteria and were predicted to be nonfunctional (FIG. 19A-2). In addition to these rational picks, four other SiRNA molecules were picked arbitrarily (SEAP-147, -500, -1113,1271). Along with SEAP-68 and -155, six randomly picked SiRNAs were used (FIG. 19B-1&2) to compare with the results of the rationally picked triggers. As shown in FIG. 19C, all four SiRNA triggers that were rationally picked to be functional using the criteria were functional in reducing the expression of the target gene SEAP. Out of those functional SiRNA triggers, the most effective trigger, SEAP 2217, has a flat internal energy profile. This result, in combination with that presented in FIG. 18B, suggests that a flat internal energy profile may also be acceptable for a functional SiRNA trigger. One trigger, out of the two triggers that were rationally designed not to be functional, was nonfunctional, whereas the other reduced the SEAP expression by approximately 50%. The efficacy of arbitrarily picked SiRNA triggers in RNAi was evaluated and only one trigger (SEAP 68) out of six molecules was functional. SEAP-155, which came from the same selection, may be moderately functional; reducing the SEAP level by approximately 50%. These results suggest that the rational design of SiRNA triggers using these criteria may be reliable in indentifying functional SiRNA triggers.

Next, the minimal effective concentration of three SiRNA triggers designed for SEAP was investigated. At a fixed 100 nM concentration, the degree of effectiveness of these three triggers in silencing SEAP expression was different (FIG. 19). As shown in FIG. 20, the minimal concentration required by three triggers to silence the gene varied 0.5, 2.5 and greater than 100 nM for SEAP-2217, -1035, and -1070, respectively. With SEAP-2217 and -1035, the magnitude of gene silencing remained constant above the minimum effective concentration. These results suggest that certain SiRNA triggers that are effective at high concentration (100 nM) may not be functional at low concentrations. Hence, the identification of optimally effective SiRNA triggers, facilitated by the guidelines outlined here, may be valuable in developing SiRNA based therapeutics. Such effective SiRNA triggers may cut down the effective dose requirement.

It appears that in certain instances SiRNA triggers with low calculated Tₘ values may be functional triggers, provided the internal energy criteria are met. Target sites with low melting temperatures may be common within mRNAs that are low in G/C content. Consequently, the probability of success in identifying functional SiRNA molecules by random picking may be high. However, as indicated in FIG. 50, the success of identifying arbitrarily designed SiRNA molecules in this work does not correlate with the G/C content of mRNA.

Certain criteria of SiRNA molecules, Tₘ in the range of 55° C., and a bell-shaped or flat internal stability profile, that promote RNAi emerged from the initial analysis of a set of naturally occurring miRNA molecules. Subsequently, the existence of these criteria in functional SiRNA molecules that were designed arbitrarily and validated experimentally was confirmed. The success of designing functional SiRNA molecules using the two criteria was demonstrated.

A possible, but nonlimiting, rationale for the benefits of the criteria follows. It is reasonable to assume that within the RISC the antisense strand of the SiRNA may come off from the duplex and subsequently anneal to the substrate mRNA. These events may be facilitated by either individual proteins or multi-domain proteins within the RISC. Under such a scenario, the two strands within an SiRNA would be expected to melt easily. In light of this view on the mechanism, the interplay of molecular forces between the two strands of an SiRNA to keep them in a dynamic environment may be important to an effective SiRNA trigger. Hence, SiRNA molecules with a relatively low Tₘ may be advantageous. SiRNA duplexes with a relatively high internal stability in the middle may keep the two strands together, while the two ends with low internal stability provide easy entry to a protein like helicase to facilitate strand separation when needed. In contrast, SiRNA duplexes that are highly stable, as characterized by a high Tₘ and high internal stability may resist the strand separation step in the RNAi mechanism and hence fail to trigger RNA interference. Recently, the participation of RNA-dependent RNA polymerase (RdRP) has been demonstrated in the RNA interference process in C. elegans (Sijen et al., 2001) and Drosophila embryo extracts (Lipardi et al., 2001). An antisense strand of SiRNA once annealed to the target mRNA may serve as a primer for an RdRP to convert mRNA into dsRNA that is degraded to generate more SiRNA molecules in situ. Even for this activity to take place, strand separation of an SiRNA molecule may be an important prerequisite.

C. Conclusion

As demonstrated experimentally, rational design of functional SiRNA triggers based on the criteria set forth herein may help facilitate target validation and other applications of RNA interference. However, there may be instances that SiRNA molecules may not necessarily adhere to these guidelines. This may be because the predictions are based on the pattern analysis of sequences alone and do not factor in the cellular environment in which the target mRNAs exist. In reality, RNA is not a linear target as treated in this analytical approach, but is folded and interacts with a host of RNA-binding proteins. Hence, these criteria are considered guidelines for a high probability for success, but may not always provide effective SIRNA molecules.

The following is a stepwise procedure for designing functional SiRNA molecules for a mRNA target sequence according to certain embodiments. Windows depicted in FIG. 21 will be helpful in understanding the process of picking effective SiRNA triggers.

1. Download the target mRNA sequence from the NCBI database (http://www.ncbi.nlm.nih.gov).

2. Open the sequence file in Oligo 5.0™ Primer Analysis Software.

3. Select the length of the primer as 19 using the dropdown menu under “Change”.

4. Search in the (lower) internal stability window for a bell-shaped region with the internal energy preferably below −10 (kcal/mol) highlighted for the 19-mer.

5. If the calculated Tₘ for that site is below 65° C. in the (upper) melting temperature window, pick the antisense strand.

6. Alternatively, pick a site with a flat internal stability profile with energy of approximately −6 to −9 kcal/mol and Tₘ of <50° C.

7. Perform a BLAST search against the EST database with the 19 nucleotide antisense to ensure only the gene of interest is targeted.

8. If these criteria are met, synthesize the SiRNA molecule with a 19 nucleotide base pair duplex consisting of two dT residues at the 3′ ends.

D. Materials and Methods

SiRNA

SiRNAs were prepared using several different methods. Certain chemically synthesized SiRNAs were synthesized using RNA phosphoramidites containing a 2′-O-TriisopropylsilylOxyMethyl (TOM) protection group from Glen Research (Sterling, Va.). Other SiRNAs were obtained from Dharmacon (Longmont, Colo.) employing 5′-Silyl-2′-bis(2-acetoethoxy)methyl (ACE) Orthoester chemistry. Synthesized SiRNAs using TOM phosphoramidites were HPLC purified, whereas those obtained from Dharmacon were used without further purification due to their high purity resulting from extremely high coupling efficiency (Scaringe, 2001). SiRNAs were annealed in an annealing buffer including 100 mM KCl, 30 mM HEPES (pH 7.5), and 2 mM MgCl₂ by heating to 75° C. for 2 minutes followed by slow cooling to ambient temperature.

Experimental Evaluation of SiRNAs in Tissue Culture

The efficiency of certain SiRNA molecules designed for each human gene target was evaluated upon transfection into cultured human cells expressing the target. The specific mRNA level of the target gene was measured following transfection. In parallel, the mRNA level of a housekeeping gene, cyclophilin, as a nonspecific target was also quantified. Changes in the cyclophilin mRNA levels with SiRNA triggers were not observed. The efficiency of SiRNA to specifically reduce the targeted mRNA was calculated as a ratio of the target mRNA to cyclophilin mRNA. SiRNA molecules that gave greater than 70% reduction of the target mRNA level were taken as functional SiRNAs.

Delivery of Nucleic Acid Triggers

Cells seeded in 96-well plates at approximately 25,000/well the previous day were transfected with different nucleic acid triggers using Lipofectamin 2000 and Opti-MEM I (from Invitrogen). Briefly, SiRNA was diluted in Opti-MEM-I in a 50 μL volume. This was mixed with an equal volume of Lipofectamin 2000 diluted 12.5-fold in OPTI-MEM I. After incubating the mixture at ambient temperature for 20 minutes, 270 μL of regular cell medium was added, and 95 μL of the solution was immediately transferred onto the cells in the plate with no media. Plates were transferred to a 37° C. incubator with 5% CO₂ for either 24 or 48 hours. To monitor the fate of SEAP transiently expressed in 293 cells, prAAV6-seap plasmid was included in the transfection mixture with and without SiRNAs.

Quantification of mRNA levels

Specific mRNA levels of cells transfected with different nucleic acid triggers were quantified using QuantiGene High Volume Kit (from Bayer) that employs a branched-DNA (b-DNA) method for nucleic acid detection according to the manufacturer's instructions. Specific detection of a given mRNA is based on its selective capture on to the microtiter plate, which is dictated by the capture probes. Probe sets that are unique to each target mRNA were designed using the ProbeDSesigner software (Bayer) according to the manufacturer's instructions. For each case, probe sets were validated using the cells expressing each message before being used for experiments.

Secreted Alkaline Phosphatase (SEAP) Assay

The SEAP gene (from pAP-1 SEAP vector from Clontech) was cloned into an adeno-associated vector (AAV # 6) upstream of the EF1-α 3′ UTR. Twenty-four hours after the transfection, 15 μL of medium from each well was transferred to a white opaque 96-well flat bottom microtiter plate, and the amount of SEAP was detected using a chemiluminescent SEAP assay (Great EscAPe SEAP assay kit form Clontech) according to the manufacturer's instructions. RLU values obtained in the presence of an SiRNA trigger were normalized to that obtained in the absence of a trigger. SiRNA molecules that gave greater than 70% reduction of the RLU level compared to the control (no SiRNA added) were taken as functional SiRNAs.

Calculation of Internal Stability Profiles and Melting Temperatures (Tₘ Values)

Internal stability profiles were calculated using the software program Oligo 5.0™ Primer Analysis Software (National Biosciences, Inc., Plymouth, Minn.), a program that is generally used for designing oligonucleotides for PCR and various nucleic acid hybridization applications. The ΔG value for each position reflects the average of all overlapping pentamer sequences of the 19 base-pair duplex. The program calculates the average ΔG value by adding ΔG values of the 4 nucleotide pairs within the pentamer. The terminal base pairs were excluded to avoid end effects and only the internal 17 nucleotide sequence was considered. Furthermore, the internal stability profiles of antisense strands were calculated using nearest neighbor calculations of DNA, not of RNA. Although the absolute values for RNA may vary, the general trends may still be valid.

Tₘ values were also calculated using the same software program according to the nearest neighbor thermodynamic values. Again, this calculation is also based on DNA and not on RNA, but the general trend for Tₘ also holds true.

IV. Certain Functional SIRNA Triggers in Mammalian Cells

It has been reported that SiRNA triggers with a 19-base pair helical region with two nucleotide 3′ extensions may be optimal triggers for gene silencing through RNA interference. These conclusions were born out based on two observations: (1) empirical design of RNA triggers to mimic cleavage products of Dicer, the enzyme involved in processing long double-stranded RNA into short interfering RNA or SiRNA; (2) Results obtained by using an in vitro system derived from Drosophila embryo extract. Certain 21 nucleotide triggers with a 19 nucleotide helicial region in fact work in silencing gene expression in mammalian cells.

The work described herein suggests that certain SiRNA triggers of variable functional anatomies work effectively in mammalian cells, a result that is different from certain results obtained using an in vitro system. Salient features of the current discovery are summarized below.

Certain SiRNA triggers with a 17-base pair RNA helical region with an antisense strand of 17 RNA nucleotides are nonfunctional in triggering RNA interference in mammalian cells. On the other hand, certain SiRNA triggers with a 17-base pair RNA helical region with an antisense strand of at least 19 RNA nucleotides are effective triggers.

Recently, it has been shown that instead of ssDNA antisense oligonucleotides, injection of dsRNA into the nematode C. elegans resulted in the loss of function of a gene to which the injected dsRNA had homology (Fire, 1998; Guo, 1995). RNAi is one manifestation of dsRNA-induced gene silencing. Other forms include post-transcriptional gene silencing (PTGS) and co-suppression observed in plants (Ketting, 2000), as well as quelling in the fungus Neurospora crassa (Reviewed in (Fire, 1999; Matzke, 2001; Waterhouse, 1999)). Nature may use the RNA silencing phenomenon to protect the cell from viral infections and from mobilization of transposons. A growing body of evidence suggests that it may also be used to regulate the expression of endogenous genes.

RNAi has been demonstrated in a host of species, including invertebrates such as hydra, planaria, trypanosomes, nematodes and insects as well as vertebrates (mouse and zebra fish) (reviewed in Bosher, 2000; Hammond, 2001; Sharp, 2001; Zamore, 2001). These studies revealed certain important aspects of RNA interference. In certain instances, it has been shown that only the gene to which the dsRNA shares homology becomes silenced (Fire, 1998; Kennerdell and Carthew, 1998). In certain instances, the dsRNA should be homologous to the exons of a gene to observe effective gene silencing, indicating that the silencing mechanism may not interfere with mRNA processing, but may occur post transcriptionally after splicing (Fire, 1998). In certain instances, the dsRNA molecule may be substantially complimentary to the coding or noncoding regions of the target mRNA. In certain instances, the dsRNA molecule may be substantially complimentary to the untranslated regions of the target mRNA, including but not limited to the 5′ and 3′ untranslated regions. In certain instances, only a few copies of the dsRNA trigger may be required to degrade mRNA present in large excess (Fire, 1998; Kennerdell and Carthew, 1998), suggesting that there may be a possible amplification step included within the molecular mechanism of RNA interference. In both worms and plants, RNA interference may spread across cell boundaries (Fire, 1998; Hamilton, 1999).

Mutants that are defective in RNA interference have been isolated in C. elegans (Grishok, 2000), Neurospora (Cogoni, 1999), Arabidopsis (Dalmay, 2000) and Chlamydomonas (Wu-Scharf, 2000). Biochemical analysis of the RNAi process was facilitated by the in vitro system derived from Drosophila embryonic cells (Hammond, 2000; Tuschl et al., 1999). Studies on the in vitro system that recapitulates the RNAi process provided some insight into the fate of the dsRNA. It was suggested that the dsRNA may be cleaved into discrete 21-23 nucleotide fragments by an ATP-dependent process which does not require the presence of target mRNA (Zamore, 2000). This suggests that the small 21-23 nucleotide dsRNA fragments may not be by products of the process, but may be intermediates in the RNAi process. These results obtained in vitro were in agreement with the previous observation of the existence of small dsRNA of 25 nucleotides in plants undergoing PTGS either by viruses or trans genes (Hamilton, 1999). These small dsRNAs found in plants undergoing PTGS included both sense and antisense strands corresponding to the silenced gene. The conversion of dsRNA into small dsRNAs was suggested in vivo in C. elegans and Drosophila as well (Parrish, 2000; Yang, 2000). The multidomain RNAse III protein Dicer may be the enzyme that processes dsRNA into 21-23 nucleotide short dsRNA (Bernstein, 2001) that are called SiRNAs (Short Interfering RNAs)(Elbashir, 2001 b). According to one of the proposed models for RNAi (Hammond, 2001; Zamore, 2001), the SiRNA molecules generated upon the cleavage of the long dsRNA become a part of a ribonucleo-protein complex called RiSC(RNA-induced silencing complex). The RISC may then find and bind to the target mRNA through a homology searching mechanism facilitated by a protein(s) within the complex and the antisense strand of the SiRNA. Once the site of homology is identified, the target mRNA may be cleaved by an endonuclease, which may be a member of RISC as well. SiRNA guided cleavage of the target RNA has been suggested in vitro (Elbashir, 2001 b). It has been suggested that the cleavage within the target RNA takes place near the center of the homology to the SiRNA.

Earlier attempts in applying relatively long (approximately 800 nts) dsRNA to initiate RNAi in mammalian cells failed (Calpan et al., 2000). However, RNAi has been observed in mouse oocytes and early embryos (Svoboda, 2000; Wianny, 2000), suggesting the possible existence of RNAi machinery in mammalian cells. The failed attempts in demonstrating RNAi in mammalian cells have been attributed to the nonspecific effects induced by long dsRNAs in mammalian cells. Certain dsRNAs are known to induce nonspecific effects in mammalian cells by activating several pathways through the rapid induction of IFNγ (Geiss, 2001; Stark, 1998). DsRNA is known to activate dsRNA-dependent protein kinase, PKR, which in turn phosphorylates and inactivates the translation factor eIF2α. The overall result is the global shut down of protein synthesis in the cell and subsequent cell death. In addition, dsRNAs may also induce the production of 2′-5′-polyadenylic acid which in turn activates the nonspecific nuclease RNase L that nonspecifically degrades RNA. The induction of nonspecific effects by dsRNA in mammalian cells may be related to the length of the dsRNA; for example, the activation of PKR may require dsRNA that is longer than 30 base pairs. The efficiency of the activation of PKR increases with the length of RNA and 85 base pairs may provide optimal activation in certain instances (Manche, 1992).

The use of short dsRNA molecules, such as SiRNA, has the potential to keep the dsRNA-induced nonspecific pathways from activating. In other words, being intermediates of the RNA interference process, SiRNA may trigger RNAi in mammalian cells. Recent results with the delivery of SiRNA into mammalian cells suggest this theory may be accurate (Caplen, 2001; Elbashir, 2001 a). These authors used SiRNA of 21-25 nucleotides to silence genes expressed either transiently or endogenously in cultured mammalian cells. The SiRNAs used in these studies were designed to have 2 nucleotide 3′-overhangs mimicking a digested RNA fragment resulting from cleavage of the ribonuclease III enzyme, Dicer. A phosphate group is present at the 5′ end of the Dicer cleavage products, yet it may not be required for a SiRNA molecule to trigger efficient RNA interference (Caplen, 2001; Elbashir, 2001 a). Furthermore, the two RNA nucleotides in the 3′-overhang contain two dT residues to presumably protect the functional SiRNA triggers from possible 3′-5′ exonuclease activities (Elbashir, 2001 a). Certain characteristics of certain SiRNA triggers have been delineated empirically. An attempt was made to understand the functional anatomy of SiRNA triggers using an in vitro system derived from Drosophila melanogaster embryo lysate (Elbashir et al., 2001). In this system, SiRNA triggers with 21 nucleotides in each strand and a 19-base pair helical region with 2 nucleotide 3′ extensions were the most efficient triggers for mediating RNA interference.

The results herein suggest that the functional anatomy of SiRNA molecules may be quite flexible in silencing genes in mammalian cells, since triggers with three possible end structures (3′-extension, 5′-extension and blunt) mediate effective gene silencing. Also, the single-stranded RNA molecules that fold into hairpin structures may be equally in triggering RNA interference in mammalian cells. These results may suggest either the mechanism by which RNA interference is mediated in mammalian cells has differences compared to the one found in lower organisms or there are some limiting factors in the in vitro system based on Drosophila embryo extract.

Materials and Methods

SiRNA

All SiRNA triggers and hairpin molecules used in this work were chemically synthesized in house using RNA phosphoramidites based on 5′-Silyl-2′-bis(2-acetoethoxy)methyl (ACE) Orthoester chemistry purchased from Dharmacon (Longmont, Colo.). After deprotection, short RNA molecules (all SiRNA triggers and short RNA hairpins) were used without further purification, due to their high purity resulting from extremely high coupling efficiency (Scaringe, 2001). The long RNA hairpin, SP-HP uucg AS-S, was purified by reverse phase high pressure liquid chromatography. Two strands of SiRNA molecules were annealed in an annealing buffer including 100 mM KCl, 30 mM HEPES (pH 7.5), and 2 mM MgCl₂ by heating to 75° C. for 2 minutes followed by slow cooling to ambient temperature. Hairpin molecules were also heated and slowly cooled down to ambient temperature in the same annealing buffer.

Delivery of Nucleic Acid Triggers

Cells seeded in 96-well plates at approximately 95% confluence were transfected with different nucleic acid triggers using Lipofectamin 2000 and Opti-MEM I (from Invitrogen). Briefly, SiRNA was diluted in Opti-MEM-I in 100 μL volume. This was mixed with an equal volume of Lipofectamin 2000 diluted 25-fold in OPTI-MEM I with SuperRNAsin at 1.4 U/μL (from Ambion) and prAAV6-seap plasmid (1 ng/μL). After incubating the mixture at ambient temperature for 5-20 minutes., 550 μL of regular cell medium was added and 100 μL of the solution was immediately transferred onto cells in the plate with no media. Plates were transferred to a 37° C. incubator with 5% CO₂ for either 24 or 48 hours. In each case, the final concentration of SiRNA triggers was 100 nM.

Secreted Alkaline Phosphatase (SEAP) Assay

Twenty-four hours after the transfection, 15 μL of medium from each well was transferred to a white opaque 96-well flat bottom microtiter plate, and the amount of SEAP was detected using a chemiluminescent SEAP assay (Great EscAPe SEAP assay kit form Clontech) according to the manufacturer's instructions.

Results and Discussion

RNA interference may be used to study gene function in mammalian cells. Two to three nucleotide 3′-extensions within SiRNA triggers for effective silencing of cognate genes in lower organisms has been reported (Elbashir et al., 2001). Effective gene silencing by hairpin SiRNA triggers in mammalian cells has been observed. In order to investigate the effect of the end structure in effective gene silencing in mammals, a reporter gene, SEcreted Alkaline Phosphatase (SEAP) was expressed under a strong CMV promoter and was used as a target gene. A plasmid expressing SEAP mixed with SiRNA triggers was transfected into HEK 293 cells. Silencing of the SEAP gene was monitored 24 hours after transfection using a chemiluminescence assay directed to detect the activity of alkaline phosphatase.

A site within the SEAP mRNA that was previously characterized to provide effective reduction of gene expression by SiRNA was chosen as the target site (FIG. 22; SiRNA trigger 2217). SiRNA triggers targeting this site were chemically synthesized with different lengths and end structures.

Variation of SiRNA Trigger Length

It has been demonstrated that SiRNA triggers with a 19 base pair helical region and two nucleotides at the 3′ overhang may be optimal for effective gene silencing in Drosophila extract (Elbashir et al., 2001). These triggers may be effective in mammalian cells as well (Caplen, 2001; Elbashir, 2001a). The effect of the change in the helical length of certain SiRNA triggers was investigated using triggers with 17, 19, 21, 23, and 25 RNA base pairs. Although these triggers carry helical regions of different lengths they have the same end structure; two dT residues as 3′ overhangs. As shown in FIG. 22, the trigger with a 17-bp helical region may be nonfunctional, whereas all other triggers appeared to be effective in silencing SEAP expression. These include triggers with 19, 21, 23 and 25 base pair helical regions. There are some minor variations in the degree of silencing; SiRNA with a 25-bp helical region had a comparable silencing effect to that of a 19-bp helical region, whereas triggers with 23 and 25 bp helical regions did not work at the same level. This result is in contrast with the length results observed in certain Drosophila extract work in which 24 and 25 nucleotide SiRNA triggers were ineffective, whereas 22 and 23 nucleotide SiRNA triggers produced 60% silencing of the targeted gene (Elbashir et al., 2001). The SiRNA trigger made up of all RNA nucleotides (SP-19-AR) with no deoxy residues at the 3′ end extensions may be as effective as the one containing two dT residues (SP-19) in silencing the targeted gene. In the case of SP-19-AR, the entire antisense strand is complimentary to the targeted mRNA, extending the complimentary region from 19 to 21 nucleotides. The effective gene silencing by all RNA SiRNA triggers is consistent with the previous observations made by Caplen et al. in mammalian cells using 21-, 22- and 23 nucleotide SiRNA triggers (Caplen, 2001).

SiRNA Triggers with Different end Structures

FIG. 23 summarizes the results of gene silencing mediated by SiRNA triggers with three possible end structures: 3′-extention, 5′-extension and blunt. The SiRNA trigger with 5′-extensions (SP-19-5′-ext and SP-19) as well as that with a blunt end (SP-19-Blunt) work equally well as that with 3′ extensions (SP-19), indicating that in certain instances the end structure of SiRNA triggers may not be important in mediating RNA interference in mammalian cells. This is a different result compared to certain work in Drosophila embryo extract (Elbashir et al., 2001). In a Drosophila based in vitro system, SiRNA triggers with 5′-extensions were nonfunctional, whereas the triggers with blunt ends produced ambiguous results, some blunt-end triggers may be functional whereas others may not be functional, in spite of all of them targeting to a common site in an mRNA. The blunt end trigger that did not mediate RNA interference (SP-19-Blunt) has only a 17 base paired RNA helical region, which may be too short to be functional as observed with SP-17 (FIG. 22). Unimolecular hairpin triggers may also be effective in triggering RNA interference in mammalian cells. In the current study, four different synthetic hairpin molecules were used (FIG. 24). Hairpin triggers with either four or eight nucleotides in the loop were synthesized. Three hairpin triggers with tetra loops were designed. The first one contains the 3′ end of the antisense strand ending at the loop (SP-HP uucg AS-S). The second has the sense strand terminating at the loop (SP-HP uucg S-AS). In the third trigger, the helical region is flanked by a loop and an internal bulge (SP-HP uucg AS-S+5′ext) providing a 5′-extension. It may be that Dicer would process these hairpin molecules into SiRNA triggers of correct lengths. Hairpin SiRNA triggers with both tetra- and octa-loops may be effective in silencing the target gene (FIG. 25), suggesting that the size of the loop may not be important for mediating RNA interference. Hairpin triggers in which the antisense strand is placed in either orientation with respect to the loop may also be used (FIG. 25; SP-HP uucg S-AS and SP-HP uucg AS-S), suggesting that in certain instances Dicer may have no preferred symmetry in processing a hairpin molecule into an SiRNA trigger. Furthermore, the hairpin molecule in which the helical region is flanked by a loop and an internal bulge (SP-HP uucg AS-S+5′ext) may be used to silence the SEAP expression, suggesting that in certain instances both ends may be processed by Dicer. This result suggests that in certain instances the end structure of a double helical RNA fragment may be insignificant to the efficacy of RNA interference in mammalian cells.

The effect of gene silencing mediated by SiRNA triggers possessing asymmetric lengths in the two strands was explored. Several series of SiRNA triggers were designed in which the length of the sense strand was kept constant and the length of the antisense strand, as well as the nature of its end structure, was changed. Sequences of SiRNA triggers belonging to eight series are listed in FIG. 26A, B and C. The results of the efficiency of silencing the SEAP expression by each of these triggers are shown in FIG. 27. In each series, SiRNA triggers carrying an antisense strand with 17 RNA nucleotides (both SP-17-as and SP-19 blunt-as) may be ineffective in silencing the targeted gene. The triggers that appear to be ineffective in silencing the SEAP gene are indicated in boxes in FIGS. 26A, B and C. In these cases, the helical length of the contiguous RNA region is 17 base pairs and that length may be too short to mediate effective RNA interference. Except for these SiRNA triggers, triggers with all other combinations of different end structures may be effective in silencing the targeted gene, which may suggest in certain instances a lack of preference for end structures in mammalian cells.

A similar series of triggers, keeping the antisense strand constant and varying the length of the sense strand, was constructed. Sequences of SiRNA triggers of eight series are illustrated in FIG. 28A, B and C, and the results of targeted gene silencing mediated by each trigger are shown in FIG. 29. Analogous to the previous results in FIGS. 23 and 27, triggers with an antisense strand with 17 RNA nucleotides may lack the ability to silence the targeted gene. However, SiRNA triggers in which the sense strand including 17 RNA nucleotides is hybridized to antisense strands that are 19 nucleotides or longer may silence the SEAP gene. These results indicate that in certain instances a short sense strand with 17 nucleotides may be acceptable for the silencing process, but a short antisense strand of the same length may not be acceptable. Thus, in certain instances minimal length, 19 RNA nucleotides, and an antisense strand of 19 nucleotides is used as an SiRNA trigger.

Certain end structures that effectively work in silencing the SEAP gene in mammalian cells are shown below.

| END STRUCTURE | EXAMPLES |
|---|---|
| Asymmetric 3′-extensions | 1. SP-17-S + SP-19-AS |
| | 2. SP-17-S + SP-19-RNA-AS |
| | 4. SP-19-S + SP-21-AS |
| | 3. SP-21-S + SP-19-AS |
| | 4. SP-21-S + SP-23-AS |
| | 5. SP-23-S + SP-21-AS |
| | 6. SP-23-S + SP-25-AS |
| | 7. SP-25-S + SP-23-AS |
| | 8. SP-19-RNA-S + SP-21-AS |
| | 9. SP-21-S + SP-19-RNA-AS |
| Symmetric 3′-extensions | 1. SP-19-S + SP-19-AS |
| | 2. SP-19-S + SP-19-RNA-AS |
| | 3. SP-21-S + SP-21-AS |
| | 4. SP-23-S + SP-23-AS |
| | 5. SP-19-RNA-S + SP-19-AS |
| | 6. SP-19-RNA-S + SP-19-RNA-AS |
| Blunt ends | 1. SP-19-S + SP-19-5′-AS |
| | 2. SP-19-RNA-S + SP-19-5′-AS |
| | 3. SP-19-5′-S + SP-19-AS |
| | 4. SP-19-5′-S + SP-19-RNA-AS |
| Blunt end and 5′ extension | 1. SP-17-S + SP-21-AS |
| | 2. SP-19-S + SP-23-AS |
| | 3. SP-21-S + SP-25-AS |
| | 4. SP-19-RNA-S + SP-23-AS |
| | 5. SP-19-Blunt-S + SP-19-AS |
| | 6. SP-19-Blunt-S + SP-19-RNA-AS |
| | 7. SP-19-Blunt-S + SP-19-5′-AS |
| | 8. SP-25-S + SP-21-AS |
| Blunt end and 3′ extension | 1. SP-23-S + SP-19-AS |
| | 2. SP-23-S + SP-19-RNA-AS |
| | 3. SP-25-S + SP-21-AS |
| | 4. SP-17-S + SP-21-AS |
| | 5. SP-19-S + SP-23-AS |
| | 6. SP-21-S + SP-25-AS |
| | 7. SP-19-RNA-S + SP-23-AS |
| | 8. SP-19-Blunt-S + SP-19-RNA-AS |
| Symmetric 5′ extension | 1. SP-19-5′-S + SP-19-5′-AS |
| | 2. SP-19-RNA-S + SP-19-AS |
| | 3. SP-25-S + SP-25-AS |
| Asymmetric 5′ extension | 1. SP-23-S + SP-25-AS |
| 5′ and 3′ extensions on one strand | 1. SP-17-S + SP-23-AS |
| | 2. SP-17-S + SP-19-5′-AS |
| | 3. SP-19-S + SP-25-AS |
| | 4. SP-21-S + SP-19-5′-AS |
| | 5. SP-23-S + SP-19-5′-AS |
| | 6. SP-25-S + SP-19-AS |
| | 7. SP-25-S + SP-19-RNA-AS |
| | 8. SP-25-S + SP-19-5′-AS |
| | 9. SP-19-RNA-AS + SP-25-AS |
| | 10. SP-19-5′-S + SP-21-AS |
| | 11. SP-19-5′-AS + SP-23-AS |
| | 12. SP-19-5′-AS + SP-25-AS |
| | 13. SP-19-Blunt-S + SP-21-AS |
| | 14. SP-19-Blunt-S + SP-23-AS |
| | 15. SP-19-Blunt-S + SP-25-AS |
| | 16. SP-17-S + SP-25-AS |

In the current work, certain anatomical structures of SiRNA triggers that efficiently work well in certain mammalian cells were different than certain triggers that work in certain in vitro systems. Caplen et al. have shown gene silencing in primary mouse embryonic fibroblasts, 293, and HeLa cells with SiRNA triggers longer than a 19 base pair helical region with 2-3 nucleotides at the 3′ extensions (Caplen, 2001). Taken together these results suggest that it may be possible that certain factors that are involved in effective utilization of double-stranded RNA with a helical region longer than 19 base pair in mammalian cells may be missing in an in vitro system. These factors may include nucleases for trimming the long double stranded RNA into effective short triggers or other proteins that help activate and/or facilitate this process.

Substitution of G-U Base Pairs

During an attempt to rationally design functional SiRNA triggers, SiRNA triggers were observed with relatively low internal stability that tend to be functional in silencing target genes in mammalian cells. In certain instances, SiRNAs containing contiguous G-C base pairs may not be optimal in eliciting RNA interference. In certain instances, one may desire SiRNA triggers that do not have higher than 4 contiguous G-C base pairs in an SIRNA trigger. An approach to make G-C base pairs less stable by substituting uridines in place of cytosines to generate G-U base pairs with a higher propensity for melting. In fact, G-U base pairs are present in certain micrORNA molecules that are also processed by Dicer, the same enzyme that processes SiRNA molecules. Hence, the inclusion of G-U base pairs in an SiRNA molecule may not affect the RNA interference process.

Two SiRNA triggers, one functional (SP-1795) and the other non functional (SP-1260), were used to explore the effect of substituting G-U base pairs for G-C base pairs (C-U substitution). In each SiRNA trigger, all cytosines in either antisense or sense strand were replaced with uridines. These strands containing uridines were combined with complimentary strands with cytosines to generate SiRNA triggers with uridines in either antisense or sense strands.

The effect of gene silencing by these SiRNA triggers is shown in FIG. 27. C-U substitution in either strand had no effect on the nonfunctional SiRNA trigger. In other words, introduction of G-U base pairs in place of G-C base pairs did not convert the particular nonfunctional SiRNA into a functional SiRNA trigger. However, the results may be different when the G-C base pairs are replaced by G-U base pairs in a functional SiRNA trigger. When cytosines in the antisense strand in certain instances were replaced with uridines, the SiRNA trigger appeared to become less functional (FIG. 27; 5^{th} bar), suggesting that in certain instances targeting through G-U wobble base pairing is undesirable to mediate RNA interference. On the other hand, substitution of cytosines with uridines in the sense strand may not affect the ability of a functional SIRNA to mediate RNA interference. Based on this observation of the tolerance of C-U substitution in the sense strand, it may be possible to improve the performance of nonfunctional SiRNA triggers. The failure of C-U substitution in the SiRNA trigger SP-1260 may be due to the extremely high number (14 out of 19 total) of contiguous G-C base pairs. If one uses a nonfunctional SiRNA with a modest number of contiguous G-C base pairs and carries out a C-U substitution in the sense strand the resulting trigger may be functional.

### CONCLUSIONS

SiRNA triggers with a 17-base pair RNA helical region with an antisense strand of greater than or equal to 19 RNA nucleotides may be effective triggers.

SiRNA triggers carrying an RNA helical region(s) greater than or equal to 19 base-pairs with different end structures may be functional in eliciting RNA interference in mammalian cells.

SiRNA triggers having a 17-base pair RNA helical region with an antisense strand of greater than or equal to 19 RNA nucleotides with different end structures may be effective triggers.

The 3′-ends may be either ribo- or two deoxy-nucleotides in certain functional SiRNA triggers.

SiRNA triggers in which a sense strand having 17 nucleotides is annealed to an antisense strand that is greater than or equal to 19 nucleotides may be functional in mammalian cells.

Unimolecular RNA molecules with the propensity to fold into hairpin structures may serve as functional SiRNA triggers in silencing gene expression in mammalian cells.

C-U substitutions in the sense strand but not in the antisense strand may be tolerated in functional SiRNA triggers.

These results suggest that SiRNA triggers with a 19-base pair helical region with two nucleotide 3′ extensions may not be the only structure with the ability to mediate highly efficient RNA interference in mammalian cells.

V. RNA Interference Using DNA Delivery

Gene silencing using RNA interference may be an attractive approach as a tool for understanding gene function and as a therapeutic approach to inhibit undesirable gene expression implicated in disease. A double stranded RNA molecule having homology to the target mRNA mediates the silencing process.

It has been demonstrated that the introduction of certain synthetic SiRNA molecules into cultured cells elicits silencing of the target gene, suggesting that the initial cleavage by the Dicer enzyme may be bypassed. Not all SiRNA triggers identified against a target gene are equally efficacious in silencing that gene, and hence screening of several triggers may be carried out. On the other hand, a relatively long dsRNA molecule cleaved by the Dicer enzyme generates several different such triggers inside the cell. The latter approach may have a higher probability of getting an effective trigger for the silencing process. Transfection of dsRNA longer than 70 base pairs has been shown to elicit cytotoxicity, and, therefore, may not be used as a functional trigger in certain instances. However, the intracellular expression of longer dsRNA may not induce cellular toxicity. Furthermore, linear dsDNA fragments having a U6 promoter upstream of either antisense or sense strands of a targeted reporter gene were constructed using PCR. These PCR fragments were intended to produce either antisense or sense strands of RNA approximately of 22 nucleotide homology to the target gene. The transfection of both types of PCR fragments (antisense and sense), along with a plasmid expressing the target reporter gene, into HEK 293 cells was able to silence the reporter gene expression.

1. One may use DNA constructs with the capacity to express RNA having significant homology to a target gene of interest in RNAi. In certain embodiments, these constructs may encode dsRNAs of 70 to 150 nucleotides.

a. RNA may be single stranded with either antisense or sense polarity to the target mRNA. DNA constructs expressing both polarities may be used in certain embodiments. However, due to the possibility of aberrant RNA generation with an orientation opposite to the promoter, even one of the constructs expressing either sense or antisense RNA may be used.

b. RNA may have a double-stranded nature due to the presence of self-complimentary regions. An example of this type of RNA is a fold-back stem loop. When RNA molecules having a double-stranded nature are expressed, a single type of DNA construct may be used.

2. In DNA constructs described in 1, the promoters that drive RNA synthesis may be of phage derived, virus-derived, pol II, or pol III type.

3. DNA constructs described in 1, may or may not contain extra nucleotides that serve additional functions such as termination of transcription or a poly A signal.

4. DNA constructs described in 1, may be either linear or circular. In the case of circular DNA, it may be a plasmid with additional genes conferring different functions such as resistance to one or more antibiotics.

5. DNA constructs described in 1, may be synthetic, derived from PCR, or derived from growing inside a host. Alternatively, DNA constructs may be derived from one or more methods described above.

6. For application in tissue culture, DNA constructs described in 1, may be introduced into cells by transfection, electroporation, or microinjection.

7. For in vivo applications in animals and humans, DNA constructs described in 1, may be delivered by:

a. Simple injection into tissues or blood or any other body fluid;

b. Under pressure;

c. Electroporation;

d. Using micro pumps;

e. Using DNA guns;

f. Orally.

8. Adjuvants or formulations that may either stabilize DNA constructs or facilitate a delivery method may be used in the delivery methods outlined in 7.

### References Cited

Bernstein, E., A. A. Caudy, S. M. Hammond, G. J. Hannon. (2001). Role for a Bidetate Ribonuclease in the Initiation Step of RNA Interference. Nature 409, 363-366.

Bosher, J. M., Labouesse, M. (2000). RNA interference: genetic wand and genetic watchdog. Nature Cell Biol. 2, E31-E35.

Burns, J. C., Friedman, T., Driever, W., Burrascano, M., Yee, J-K. (1993). Vesicular stomatitis virus G glycoprotein pseudotyped retroviral vectors: concentration to very high titer and efficient gene transfer into mammalian and nonmammalian cells. Proc. Natl. Acad. Sci. USA 90, 8033-8037.

Caplen, N. J., Fleenor, J., Fire, A., Morgan, R. A. (2000). dsRNA-mediated gene silencing in cultured Drosophila cells: a tissue culture model for the analysis of RNA interference. Gene 252, 95-105.

Caplen, N. J., S. Parrish, F. Imani, A. Fire, R. A. Morgan. (2001). Specific Inhibition of Gene Expression by Small Double-Stranded RNAs in Invertebrate and Vertebrate Systems. Proc. Natl. Acad. Sci. USA 98, 9742-9747.

Chuang, C.-F., Meyerowitz, E. M. (2000). Specific and heritable genetic interference by double-stranded RNA in Arabidopsis thaliana. Proc. Natl. Acad. Sci. USA 97, 4985-4990.

Cogoni, C., G. Macino. (1999). Gene Silencing in Neurospora crassa Requires a Protein Homologous to RNA-Dependent RNA Polymerase. Nature 399, 166-169.

Dalmay, T., A. Hamilton, S. Rudd, S. Angell, D. C. Baulcombe. (2000). An RNA-Dependent RNA Polymerase Gene in Arabidopsis is Required for Posttranscriptional Gene Silencing Mediated by a Transgene but not by a Virus. Cell 101, 543-553.

Elbashir, S. M., J. Harborth, W. Lendeckel, A. Yalcin, K. Weber, R. Tuschl. (2001 a). Duplexes of 21-Nucleotide RNAs Mediate RNA Interference in Cultured Mammalian Cells. Nature 411, 494-498.

Elbashir, S. M., Martinez, J., Patkaniowska, A., Lendeckel, W. and Tuschl, T. (2001). Functional anatomy of SiRNAs for mediating efficient RNAi in Drosophila melanogaster embryo lysate. EMBO J. 20, 6877-6888.

Elbashir, S. M., W. Lendeckel, R. Tuschl. (2001 b). RNA Interference is Mediated by 21- and 22-Nucleotide RNAs. Genes and Development 15, 188-200.

Fire, A. (1999). RNA-triggered gene silencing. Trends in Genetics 15, 358-363.

Fire, A., Xu, S-Q., Montgomery, M. K., Kostas, S. A., Driver, S. E., Mello, C. C. (1998). Potent and specific genetic interference by double-stranded RNA in Caenorhabditis elegans. Nature 391, 806-810.

Geiss, G., Jin, G., Guo, J., Bumgarner, R., Katze, M. G., Sen, G. C. (2001). A comprehensive new view of regulation of gene expression by double-stranded RNA-mediated cell signaling. J. Biol. Chem. 276, 30178-30182.

Green, L. S., Jellinek, D., Bell, C., Beebe, L. A., Feistner, B. D., Gill, S. C., Juker, F. M., Janjic, N. (1995). Nuclease-resistant nucleic acid ligands to vascular permeability factor/vascular endothelial growth factor. Curr. Biol. 2, 683-695.

Good, P. D., Kirkos, A. J., Li, S. X. L., Bertrand, E., Lee, N. S., Giver, L., Ellington, A., Zaia, J. A., Rossi, J. J., Engelke, D. R. (1997). Expression of small, therapeutic RNAs in human cell nuclei. Gene Ther. 4, 45-54.

Grishok, A., H. Tabara, C. Mello. (2000). Genetic Requirements for Inheritance of RNAi in C. elegans. Science 287, 2494-2496.

Guo, S., Kemphues, K. J. (1995). par-1, a gene required for establishing polarity in C. elegans embryos, encodes a ser/Thr kinase that is asymmetrically distributed. Cell 81, 611-620.

Hamilton, A. J., D. C. Baulcombe. (1999). A Species of Small Antisense RNA in Posttranscriptional Gene Silencing in Plants. Science 286, 950-952.

Hammond, S. M., Caudy, A. A., Hannon, G. J. (2001). Post-transcriptional gene silencing by double-stranded RNA. Nature Rev. Genetics 2, 110-119.

Hammond, S. M., E. Bernstein, D. Beach, G. J. Hannon. (2000). An RNA-Directed Nuclease Mediates Post-Transcriptional Gene Silencing in Drosophila Cells. Nature 404, 293-296.

http://www.dharmacon.com/tech/tech003.html.

http://www.mpibpc.gwdg.de/abteilungen/100/105/SiRNA.html.

Hutvagner, G., McLachlan, J., Pasquinelli, A. E., Balint, E., Tuschl, T. and Zamore, P. D. (2001). A cellular function for the RNA-interference enzyme Dicer in the maturation of the let-7 small temporal RNA. Science 293, 834838.

Kennerdell, J. R. and Carthew, R. W. (1998). Use of dsRNA-Mediated Genetic Interference to Demonstrate that frizzed and frizzed 2 Act in the Wingless Pathway. Cell 95, 1017-1026.

Ketting, R. F., Plasterk, R. H. A. (2000). A Genetic Link Between Co-Suppression and RNA Interference in C. elegans. Nature 404, 296-298.

Kruger, M., Beger, C., Li, Q-X., Welch, P. J., Tritz, R., Leavitt, M., Barber, J. R., Wong-Staal, F. (2000). Identification of eIF2Bg and eIF2g as cofactors of hepatitis C virus internal ribosome entry site-mediated translation using a functional genomics approach. Proc. Natl. Acad. Sci. USA 97, 8566-8571.

Landau, N. R., Littman, D. R. (1992). Packaging system for rapid production of murine leukemia virus vectors with variable tropism. J. Virol. 66, 5110-5113.

Largos-Quintana, M., Rauhut, R., Lendeckel, W. and Tuschl, T. (2001). Identification of novel genes coding for small expressed RNAs. Science 294, 853-858.

Lau, N. C., Lim, L. P., Weinstein, E. G. and Bartel, D. P. (2001). An abundant class of tiny RNAs with probable regulatory roles in Caenorhabditis elegans. Science 294, 858-862.

Lee, R. C. and Ambros, V. (2001). An extensive class of small RNA in Caenorhabditis elegans. Science 294, 862-864.

Li, Q.-X., Robbins, Welch, P. J., Won-Staal, F. Barber, J. R. (2000). A novel functional genomics approach identifies mTERT as a repressor of fibroblast transformation. Nucleic Acids Res. 28, 2605-2612.

Lin. Y., Qiu, Q., Gill, S. C., Jayasena, S. D. (1994). Modified RNA sequence pools for in vitro selection. Nucleic Acids Res. 22(24), 5229-34.

Lipardi, C., Wei, Q. and Paterson, B. M. (2001). RNAi as random degradative PCR: SiRNA primers convert mRNA into dsRNAs that are degraded to generate new SiRNAs. Cell 107, 297-307.

Manche, L., Green, S. R., Schmedt, C., Mathews, M. B. (1992). Interaction between double-stranded RNA regulators and the protein kinase DAI. Mol. Cell Biol. 12, 5238-5248.

Matzke, M. A., Matzke, A. J. M., Pruss, G. J., Vance, V. B. (2001). RNA-based silencing strategies in plants. Curr. Opin. in Gen. & Dev. 11, 221-227.

Milligan, J. F., Groebe, D. R., Witherell, G. W., Ullenbeck, O. C. (1987). Oligoribonucleotide sythesis using T7 RNA polymerase and synthetic DNA templates. Nucleic Acid Res. 15, 8783-8798.

Murray, J. A. H., Crockett, N. (1992). Anitsense Techniques: An overview. In Antisense RNA & DNA (Murray, J. A. H., ed.), Vol.11, pp.1-49. Wiley-Liss, Inc., New York.

Ngo, H., C. Tschudi, K. Gull, E. Ullu. (1998). Double-Stranded RNA Induces mRNA Degradation in Trypanosoma brucei. PNAS 95, 14687-14692.

Pagratis, N. C., Bell, C., Chang, Y.-F., Jennings, S., Fitzwater, T., Jellinek, D., Dang, C. (1997). Potent 2′-amino-, and 2′-fluoro-2′deoxyribonucleotide RNA inhibitors of keratinocyte growth factor. Nat. Biotechnol. 15, 68-73.

Parrish, S., J. Fleenor, S. Xu, C. Mello, A. Fire. (2000). Functional Anatomy of a dsRNA Trigger: Differential Requirements for the Two Trigger Strands in RNA Interference. Molecular Cell 6, 1077-1087.

Piccin, A., A. Salameh, C. Benna, F. Sandrelli, G. Mazzotta, M. Zordan, E. Rosato, C. P. Kyriacou, R. Costa. (2001). Efficient and Heritable Functional Knock-Out of an Adult Phenotype in Drosophila using a GAL4-Driven Hairpin RNA Incorporating a Heterologous Spacer. Nucleic Acids Research 29.

Pieken, W. A., Olsen, D. B., Benseler, F., Aurup, H., Eckstein, F. (1991). Kinetic characterization of ribonuclease-resistant 2′-modified hammerhead ribozymes. Science 253, 314-317.

Rasquinelli, A., E., B. J. Reinhart, F. Slack, M. Q. Martindale, M. I. Kuroda, B. Maller, D. C. Hayward, E. E. Ball, B. Degnan, P. Muller, J. Spring, A. Srinivasan, M. Fishman, J. Finnerty, J. Corbo, M. Levine, P. Leahy, E. Davidson, G. Ruvkun. (2000). Conservation of the Sequence and Temporal Expression of let-7 Heterochronic Regulatory RNA. Nature 408, 86-88.

Scaringe, S. A. (2001). RNA oligonucleotide synthesis via 5′-Silyl-2′-Orthoester chemistry. Methods 23, 206-217.

Sharp, P. A. (2001). RNA Interference-2001. Genes & Development 15, 485-490.

Sijen, T., Fleenor, J., Simmer, F., Thijssen, K. L., Parrish, S., Timmons, L. and Plasterk, R. H. A. (2001). On the role of RNA amplification in dsRNA-triggered gene silencing. Cell 107, 465-476.

Stark, G. R., Kerr, I. M., Williams, B. R. G., Silverman, R. H., Schreiber, R. D. (1998). How cells respond to interferons. Annu. Rev. Biochem. 67, 227-264.

Svoboda, P., Stein, P., Hayashi, H., Schultz, R. M. (2000). Selective reduction of dormant maternal mRNA in mouse oocytes by RNA interference. Development 127, 4147-4156.

Tavernarakis, N., S. L. Wang, M. Dorovkov, A. Ryazanov, M. Driscoll. (2000). Heritable and Inducible Genetic Interference by Double-Stranded RNA Encoded by Transgenes. Nature Genetics 24, 180-183.

Tuschl, T., Zamore, P. D., Lehmann, R., Bartel, D. P. and Sharp, P. A. (1999). Targeted mRNA Degradation by Double-Stranded RNA in vitro. Genes & Development 13, 3191-3197.

Voinnet, O. (2001). RNA Silencing as a Plant Immune System Against Viruses. Trends in Genetics 17, 449-459.

Wang, Z., Morris, J. C., Drew, M. E., Englund, P. T., (2000). Inhibition of Trypanosoma brucei gene expression by RNA interference using an integratable vector with opposing T7 promoters. J. Biol. Chem. 275, 40174-40179.

Waterhouse, P. M., Smith, N. A., Wang, M-B. (1999). Virus resistance and gene silencing: killing the messenger. Trends in Plant Sci. 4, 452-457.

Waterhouse, P. M., Wang, M-B., Lough, T. (2001). Gene silencing as an adaptive defense against viruses. Nature 411, 834-842.

Wianny, F., M. Zernicka-Goetz. (2000). Specific Interference with Gene Function by Double-Stranded RNA in Early Mouse Development. Nature Cell Biology 2, 70-75.

Wu-Scharf, D., Jeong, B., Zhang, C., Cerutti, H. (2000). Transgene and transposon silencing in Chlamydomonas reinhardtii by a DEAH-Box RNA helicase. Science 290, 1159-1162.

Yang, D., Lu, H., Erickson, J. W. (2000). Evidence that processes small dsRNA may mediate sequence-specific mRNA degradation during RNAi in Drosophila embryos. Current Biology 10, 1191-1200.

Zamore, P. D. (2001). RNA interference: listening to the sound of silence. Nat. Struct. Biol. 8, 746-750.

Zamore, P. D., T. Tuschl, P. A. Sharp, D. P. Bartel. (2000). RNAi: Double-Stranded RNA Directs the ATP-Dependent Cleavage of mRNA at 21 to 23. Nucleotide Intervals. Cell 101, 25-33.

We claim:

1. A method of decreasing the level of a target mRNA in a host cell, comprising
a) contacting the host cell with a double-stranded RNA molecule, wherein the double-stranded RNA molecule comprises a sequence substantially complementary to at least a portion of the target mRNA, and wherein the double-stranded RNA molecule further comprises at least one chemical modification;
b) incubating the host cell under conditions whereby RNA interference occurs;
c) thereby decreasing the level of the target mRNA.

2. The method of claim 1, wherein the at least one chemical modification is selected from 2′-F, 2′-OMe, and 2′-deoxy.

3. A method of decreasing the level of a target mRNA in a host cell, comprising
a) delivering to a host cell a vector comprising a first nucleic acid sequence and a second nucleic acid sequence, wherein
i) the first nucleic acid sequence encodes a first RNA molecule comprising a first RNA sequence that is substantially complementary to at least a portion of the target mRNA; and
ii) the second nucleic acid sequence encodes a second RNA molecule comprising a second RNA sequence that is substantially identical to at least a portion of the target mRNA;
b) incubating the host cell under conditions
i) that allow transcription of the first nucleic acid sequence and the second nucleic acid sequence; and
ii) that allow RNA interference to occur;
c) thereby decreasing the level of the target mRNA.

4. The method of claim 3, wherein the first RNA sequence and the second RNA sequence are each longer than about 70 nucleotides.

5. The method of claim 3, wherein the vector further comprises at least one promoter selected from a phage promoter, a viral promoter, a pol II promoter, and a pol III promoter.

6. A method of selecting a double-stranded RNA molecule, comprising
a) inputting a target mRNA sequence into Oligo 5.0™ Primer Analysis software;
b) selecting 19 for the primer length;
c) identifying a primer in the internal stability window, wherein the primer has a bell-shaped internal energy profile, a maximum internal energy of less than −10 kcal/mol, and a melting temperature below 65° C.;
d) performing a BLAST search on the primer against an EST database; and
e) synthesizing a double-stranded RNA molecule comprising a first RNA strand comprising a first RNA sequence that is substantially identical to the nucleotide sequence of the primer and a second RNA strand comprising a second RNA sequence that is substantially complementary to the nucleotide sequence of the primer.

7. A method of selecting a double-stranded RNA molecule, comprising
a) inputting a target mRNA sequence into Oligo 5.0™ Primer Analysis software;
b) selecting 19 for the primer length;
c) identifying a primer in the internal stability window, wherein the primer has a substantially flat internal energy profile, an internal energy of between −6 and -9 kcal/mol, and a melting temperature below 50° C.;
d) performing a BLAST search on the primer against an EST database; and
e) synthesizing a double-stranded RNA molecule comprising a first RNA strand comprising a first RNA sequence that is substantially identical to the nucleotide sequence of the primer and a second RNA strand comprising a second RNA sequence that is substantially complementary to the nucleotide sequence of the primer.

8. A method of decreasing the level of a target mRNA in a mammalian host cell, comprising
a) contacting the mammalian host cell with an RNA hairpin molecule, wherein the RNA hairpin molecule comprises a first region, a second region, and a third region, wherein
i) the first region comprises a sequence that is substantially identical to at least a portion of the target mRNA;
ii) the third region comprises a sequence that is substantially complementary to at least a portion of the first region; and
iii) wherein the first region and the third region hybridize, thereby forming an RNA hairpin molecule; and
b) incubating the mammalian host cell under conditions whereby RNA interference occurs;
thereby decreasing the level of the target mRNA in the mammalian host cell.

9. A method of decreasing the level of a target mRNA in a host cell, comprising
a) delivering to a host cell a vector comprising a nucleic acid sequence, wherein the nucleic acid sequence encodes an RNA hairpin molecule, and wherein the RNA hairpin molecule comprises a first region, a second region, and a third region, wherein
i) the first region comprises a sequence that is substantially identical to at least a portion of the target mRNA;
ii) the third region comprises a sequence that is substantially complementary to at least a portion of the first region; and
iii) wherein the first region and the third region hybridize, thereby forming an RNA hairpin molecule; and
b) incubating the host cell under conditions that
i) allow transcription of the nucleic acid sequence; and
ii) allow RNA interference to occur;
c) thereby decreasing the level of the target mRNA.

10. A method of constructing a library of RNA hairpin molecules comprising
a) synthesizing a plurality of single-stranded DNA hairpin templates, wherein each single-stranded DNA hairpin template comprises a first region, a second region, and a third region, wherein
i) the first region comprises an RNA polymerase promoter sequence;
ii) the second region comprises a random nucleotide sequence having between 5 and 500 nucleotides; and
iii) the third region comprises a first nucleotide sequence, a second nucleotide sequence, and a third nucleotide sequence, wherein the first nucleotide sequence hybridizes to the third nucleotide sequence, thereby forming a single-stranded DNA hairpin template;
b) extending the 3′ end of the third nucleotide sequence of each of the plurality of single-stranded DNA hairpin templates to form a plurality of double-stranded DNA hairpin templates;
c) amplifying the plurality of double-stranded DNA hairpin templates to form a plurality of double-stranded DNA templates;
d) transcribing the plurality of double-stranded DNA templates to form a library of RNA hairpin molecules.

11. A method of identifying a target gene comprising
a) forming an array comprising a plurality of positions, wherein each position comprises at least one mammalian cell;
b) contacting the at least one mammalian cell at each position with at least one RNA hairpin molecule;
c) incubating the at least one mammalian cell under conditions that allow RNA interference to occur;
c) selecting an at least one mammalian cell exhibiting at least one biological endpoint;
d) identifying the at least one RNA hairpin molecule associated with the plurality of cells exhibiting at least one biological endpoint; and
e) performing a BLAST search on the nucleic acid sequence of the at least one RNA hairpin molecule, thereby identifying the target gene.

12. A library comprising a plurality of RNA hairpin molecules, wherein each RNA hairpin molecule comprises a first region, a second region, and a third region, wherein the first region comprises a random nucleotide sequence having between 5 and 500 nucleotides and the third region comprises a nucleotide sequence that is substantially complementary to at least a portion of the first region.


SEQUENCE LISTING (151 sequences)

SEQ ID NO: 1 - RNA, 25 nt, Artificial Sequence
  Description of Artificial Sequence Synthetic oligonucleotide
cagaaacggaugcucccaggccaag

SEQ ID NO: 2 - DNA, 23 bp, Artificial Sequence
  Description of Combined DNA/RNA Molecule Synthetic oligonucleotide
cagaaacggaugcucccaggctt

SEQ ID NO: 3 - DNA, 23 bp, Artificial Sequence
  Description of Combined DNA/RNA Molecule Synthetic oligonucleotide
gccugggagcauccguuucugtt

SEQ ID NO: 4 - DNA, 21 bp, Artificial Sequence
  Description of Combined DNA/RNA Molecule Synthetic oligonucleotide
cagaaacggaugcucccagtt

SEQ ID NO: 5 - DNA, 21 bp, Artificial Sequence
  Description of Combined DNA/RNA Molecule Synthetic oligonucleotide
cugggagcauccguuucugtt

SEQ ID NO: 6 - DNA, 25 bp, Artificial Sequence
  Description of Combined DNA/RNA Molecule Synthetic oligonucleotide
cagugucucuugaaccagttcccag

SEQ ID NO: 7 - DNA, 23 bp, Artificial Sequence
  Description of Combined DNA/RNA Molecule Synthetic oligonucleotide
cagugucucuugaaccaguuctt

SEQ ID NO: 8 - DNA, 23 bp, Artificial Sequence
  Description of Combined DNA/RNA Molecule Synthetic oligonucleotide
gaacugguucaagagacacugtt

SEQ ID NO: 9 - DNA, 21 bp, Artificial Sequence
  Description of Combined DNA/RNA Molecule Synthetic oligonucleotide
cagugucucuugaaccagutt

SEQ ID NO: 10 - DNA, 21 bp, Artificial Sequence
  Description of Combined DNA/RNA Molecule Synthetic oligonucleotide
acugguucaagagacacugtt

SEQ ID NO: 11 - DNA, 25 bp, Artificial Sequence
  Description of Artificial Sequence Synthetic oligonucleotide
gtgggaagaaggtggcagtgaactc

SEQ ID NO: 12 - DNA, 23 bp, Artificial Sequence
  Description of Combined DNA/RNA Molecule Synthetic oligonucleotide
gugggaagaagguggcagugatt

SEQ ID NO: 13 - DNA, 23 bp, Artificial Sequence
  Description of Combined DNA/RNA Molecule Synthetic oligonucleotide
ucacugccaccuucuucccactt

SEQ ID NO: 14 - DNA, 21 bp, Artificial Sequence
  Description of Combined DNA/RNA Molecule Synthetic oligonucleotide
gugggaagaagguggcagutt

SEQ ID NO: 15 - DNA, 21 bp, Artificial Sequence
  Description of Combined DNA/RNA Molecule Synthetic oligonucleotide
acugccaccuucuucccactt

SEQ ID NO: 16 - DNA, 46 bp, Artificial Sequence
  Description of Combined DNA/RNA Molecule Synthetic oligonucleotide
cagaaacggaugcucccagttttttcugggagcauccguuucugtt

SEQ ID NO: 17 - RNA, 49 nt, Artificial Sequence
  Description of Artificial Sequence Synthetic oligonucleotide
gggacagaaacggaugcucccaguuuuuucuggagcauccguuucuguu

SEQ ID NO: 18 - DNA, 46 bp, Artificial Sequence
  Description of Combined DNA/RNA Molecule Synthetic oligonucleotide
cagugucucuugaaccaguttttttacugguucaagagacacugtt

SEQ ID NO: 19 - RNA, 50 nt, Artificial Sequence
  Description of Artificial Sequence Synthetic oligonucleotide
gggacagugucucuugaaccaguuuuuuuacugguucaagagacacuguu

SEQ ID NO: 20 - DNA, 46 bp, Artificial Sequence
  Description of Combined DNA/RNA Molecule Synthetic oligonucleotide
gugggaagaagguggcaguttttttacugccaccuucuucccactt

SEQ ID NO: 21 - RNA, 50 nt, Artificial Sequence
  Description of Artificial Sequence Synthetic oligonucleotide
gggagugggaagaagguggcaguuuuuuuacugccaccuucuucccacuu

SEQ ID NO: 22 - DNA, 59 bp, Artificial Sequence
  Description of Artificial Sequence Synthetic oligonucleotide
tcttgtcagggcgaggctgttaacccttactgtgctgtgctcgtcaaagagtatgtcga

SEQ ID NO: 23 - DNA, 19 bp, Artificial Sequence
  Description of Artificial Sequence Synthetic oligonucleotide
ggctgttaacccttactgt

SEQ ID NO: 24 - DNA, 19 bp, Artificial Sequence
  Description of Artificial Sequence Synthetic oligonucleotide
gctgttaacccttactgtg

SEQ ID NO: 25 - DNA, 21 bp, Artificial Sequence
  Description of Combined DNA/RNA Molecule Synthetic oligonucleotide
gaacaugaucgucucaguctt

SEQ ID NO: 26 - DNA, 21 bp, Artificial Sequence
  Description of Combined DNA/RNA Molecule Synthetic oligonucleotide
gacugagacgaucauguuctt

SEQ ID NO: 27 - DNA, 21 bp, Artificial Sequence
  Description of Combined DNA/RNA Molecule Synthetic oligonucleotide
acugucuggcacauguuugtt

SEQ ID NO: 28 - DNA, 21 bp, Artificial Sequence
  Description of Combined DNA/RNA Molecule Synthetic oligonucleotide
caaacaugugccagacagutt

SEQ ID NO: 29 - DNA, 21 bp, Artificial Sequence
  Description of Combined DNA/RNA Molecule Synthetic oligonucleotide
ugacaacgggccacaacuctt

SEQ ID NO: 30 - DNA, 21 bp, Artificial Sequence
  Description of Combined DNA/RNA Molecule Synthetic oligonucleotide
gaguuguggcccguugucatt

SEQ ID NO: 31 - DNA, 21 bp, Artificial Sequence
  Description of Combined DNA/RNA Molecule Synthetic oligonucleotide
acucucugacauacaucactt

SEQ ID NO: 32 - DNA, 21 bp, Artificial Sequence
  Description of Combined DNA/RNA Molecule Synthetic oligonucleotide
gugauguaugucagagagutt

SEQ ID NO: 33 - DNA, 21 bp, Artificial Sequence
  Description of Combined DNA/RNA Molecule Synthetic oligonucleotide
cuggugagcuggcccgccctt

SEQ ID NO: 34 - DNA, 21 bp, Artificial Sequence
  Description of Combined DNA/RNA Molecule Synthetic oligonucleotide
gggcgggccagcucaccagtt

SEQ ID NO: 35 - DNA, 21 bp, Artificial Sequence
  Description of Combined DNA/RNA Molecule Synthetic oligonucleotide
aacauccggccgggcgccgtt

SEQ ID NO: 36 - DNA, 21 bp, Artificial Sequence
  Description of Combined DNA/RNA Molecule Synthetic oligonucleotide
cggcgcccggccggauguutt

SEQ ID NO: 37 - DNA, 21 bp, Artificial Sequence
  Description of Combined DNA/RNA Molecule Synthetic oligonucleotide
cagaaguccggguucuccutt

SEQ ID NO: 38 - DNA, 21 bp, Artificial Sequence
  Description of Combined DNA/RNA Molecule Synthetic oligonucleotide
aggagaacccggacuucugtt

SEQ ID NO: 39 - DNA, 21 bp, Artificial Sequence
  Description of Combined DNA/RNA Molecule Synthetic oligonucleotide
accucaucaucuuccugggtt

SEQ ID NO: 40 - DNA, 21 bp, Artificial Sequence
  Description of Combined DNA/RNA Molecule Synthetic oligonucleotide
cccaggaagaugaugaggutt

SEQ ID NO: 41 - DNA, 21 bp, Artificial Sequence
  Description of Combined DNA/RNA Molecule Synthetic oligonucleotide
ggcgaggcgugcugcacuctt

SEQ ID NO: 42 - DNA, 21 bp, Artificial Sequence
  Description of Combined DNA/RNA Molecule Synthetic oligonucleotide
gagugcagcacgccucgcctt

SEQ ID NO: 43 - DNA, 21 bp, Artificial Sequence
  Description of Combined DNA/RNA Molecule Synthetic oligonucleotide
gugggaguggucggcagugtt

SEQ ID NO: 44 - DNA, 21 bp, Artificial Sequence
  Description of Combined DNA/RNA Molecule Synthetic oligonucleotide
cacugccgaccacucccactt

SEQ ID NO: 45 - DNA, 21 bp, Artificial Sequence
  Description of Combined DNA/RNA Molecule Synthetic oligonucleotide
ucuggugcaggaauggcugtt

SEQ ID NO: 46 - DNA, 21 bp, Artificial Sequence
  Description of Combined DNA/RNA Molecule Synthetic oligonucleotide
cagccauuccugcaccagatt

SEQ ID NO: 47 - DNA, 21 bp, Artificial Sequence
  Description of Combined DNA/RNA Molecule Synthetic oligonucleotide
cggauguuaccgagagcgatt

SEQ ID NO: 48 - DNA, 21 bp, Artificial Sequence
  Description of Combined DNA/RNA Molecule Synthetic oligonucleotide
ucgcucucgguaacauccgtt

SEQ ID NO: 49 - DNA, 156 bp, Artificial Sequence
  Description of Artificial Sequence Synthetic oligonucleotide
atgtcgccatttcttcggattggcttgtccaactttgactgcgggtcctgccagtcttgtcagggcgaggctgttaacccttactgtgctgtgctcgtcaaagagtatgtcgaatcagagaacgggcagatgtatatccagaaaaagcctaccatg

SEQ ID NO: 50 - protein, 52 aa, Artificial Sequence
  Description of Artificial Sequence Synthetic peptide
MSPFLRIGLSNFDCGSCQSCQGEAVNPYCAVLVKEYVESENGQMYIQKKPTM

SEQ ID NO: 51 - DNA, 19 bp, Artificial Sequence
  Description of Combined DNA/RNA Molecule Synthetic oligonucleotide
ugauguaugucagagagtt

SEQ ID NO: 52 - DNA, 19 bp, Artificial Sequence
  Description of Combined DNA/RNA Molecule Synthetic oligonucleotide
cucucugacauacaucatt

SEQ ID NO: 53 - DNA, 21 bp, Artificial Sequence
  Description of Combined DNA/RNA Molecule Synthetic oligonucleotide
ugugauguaugucagagagtt

SEQ ID NO: 54 - DNA, 21 bp, Artificial Sequence
  Description of Combined DNA/RNA Molecule Synthetic oligonucleotide
cacucucugacauacaucatt

SEQ ID NO: 55 - DNA, 23 bp, Artificial Sequence
  Description of Combined DNA/RNA Molecule Synthetic oligonucleotide
cugugauguaugucagagagutt

SEQ ID NO: 56 - DNA, 23 bp, Artificial Sequence
  Description of Combined DNA/RNA Molecule Synthetic oligonucleotide
acacucucugacauacaucagtt

SEQ ID NO: 57 - DNA, 27 bp, Artificial Sequence
  Description of Combined DNA/RNA Molecule Synthetic oligonucleotide
gcugugauguaugucagagaguguutt

SEQ ID NO: 58 - DNA, 27 bp, Artificial Sequence
  Description of Combined DNA/RNA Molecule Synthetic oligonucleotide
aacacucucugacauacaucacagctt

SEQ ID NO: 59 - RNA, 21 nt, Artificial Sequence
  Description of Artificial Sequence Synthetic oligonucleotide
gugauguaugucagagagugu

SEQ ID NO: 60 - RNA, 21 nt, Artificial Sequence
  Description of Artificial Sequence Synthetic oligonucleotide
acucucugacauacaucacag

SEQ ID NO: 61 - RNA, 19 nt, Artificial Sequence
  Description of Artificial Sequence Synthetic oligonucleotide
gugauguaugucagagagu

SEQ ID NO: 62 - RNA, 19 nt, Artificial Sequence
  Description of Artificial Sequence Synthetic oligonucleotide
acucucugacauacaucac

SEQ ID NO: 63 - RNA, 21 nt, Artificial Sequence
  Description of Artificial Sequence Synthetic oligonucleotide
cugugauguaugucagagagu

SEQ ID NO: 64 - RNA, 21 nt, Artificial Sequence
  Description of Artificial Sequence Synthetic oligonucleotide
acacucucugacauacaucac

SEQ ID NO: 65 - RNA, 23 nt, Artificial Sequence
  Description of Artificial Sequence Synthetic oligonucleotide
cugugauguaugucagagagugu

SEQ ID NO: 66 - RNA, 23 nt, Artificial Sequence
  Description of Artificial Sequence Synthetic oligonucleotide
acacucucugacauacaucacag

SEQ ID NO: 67 - RNA, 42 nt, Artificial Sequence
  Description of Artificial Sequence Synthetic oligonucleotide
acucucugacauacaucacuucggugauguaugucagagagu

SEQ ID NO: 68 - RNA, 42 nt, Artificial Sequence
  Description of Artificial Sequence Synthetic oligonucleotide
gugauguaugucagagaguuucgacucucugacauacaucac

SEQ ID NO: 69 - RNA, 54 nt, Artificial Sequence
  Description of Artificial Sequence Synthetic oligonucleotide
gggaaaacucucugacauacaucacuucggugauguaugucagagaguaaaccc

SEQ ID NO: 70 - RNA, 46 nt, Artificial Sequence
  Description of Artificial Sequence Synthetic oligonucleotide
acucucugacauacaucacagugcugagugauguaugucagagagu

SEQ ID NO: 71 - DNA, 23 bp, Artificial Sequence
  Description of Combined DNA/RNA Molecule Synthetic oligonucleotide
cacucucugacauacaucacatt

SEQ ID NO: 72 - DNA, 25 bp, Artificial Sequence
  Description of Combined DNA/RNA Molecule Synthetic oligonucleotide
acacucucugacauacaucacagtt

SEQ ID NO: 73 - RNA, 21 nt, Artificial Sequence
  Description of Artificial Sequence Synthetic oligonucleotide
acacucucugacauacaucac

SEQ ID NO: 74 - DNA, 23 bp, Artificial Sequence
  Description of Combined DNA/RNA Molecule Synthetic oligonucleotide
ugugauguaugucagagagugtt

SEQ ID NO: 75 - DNA, 25 bp, Artificial Sequence
  Description of Combined DNA/RNA Molecule Synthetic oligonucleotide
cugugauguaugucagagagugutt

SEQ ID NO: 76 - DNA, 21 bp, Artificial Sequence
  Description of Combined DNA/RNA Molecule Synthetic oligonucleotide
cugugauguaugucagagagt

SEQ ID NO: 77 - DNA, 19 bp, Artificial Sequence
  Description of Combined DNA/RNA Molecule Synthetic oligonucleotide
gugauguaugucagagagt

SEQ ID NO: 78 - DNA, 21 bp, Artificial Sequence
  Description of Combined DNA/RNA Molecule Synthetic oligonucleotide
aacauuugguuggguguugtt

SEQ ID NO: 79 - DNA, 21 bp, Artificial Sequence
  Description of Combined DNA/RNA Molecule Synthetic oligonucleotide
ugguguuugguuggauguutt

SEQ ID NO: 80 - DNA, 21 bp, Artificial Sequence
  Description of Combined DNA/RNA Molecule Synthetic oligonucleotide
ugauaauggguuauaauuutt

SEQ ID NO: 81 - DNA, 21 bp, Artificial Sequence
  Description of Combined DNA/RNA Molecule Synthetic oligonucleotide
gaguugugguuuguuguuatt

SEQ ID NO: 82 - DNA, 25 bp, Artificial Sequence
  Description of Artificial Sequence Synthetic oligonucleotide
cagaaacggatgctcccaggccaag

SEQ ID NO: 83 - DNA, 25 bp, Artificial Sequence
  Description of Artificial Sequence Synthetic oligonucleotide
cagtgtctcttgaaccagttcccag

SEQ ID NO: 84 - DNA, 22 bp, Artificial Sequence
  Description of Combined DNA/RNA Molecule Synthetic oligonucleotide
ggcuguuaacccuuacugugtt

SEQ ID NO: 85 - DNA, 22 bp, Artificial Sequence
  Description of Combined DNA/RNA Molecule Synthetic oligonucleotide
cacaguaaggguuaacagcctt

SEQ ID NO: 86 - DNA, 22 bp, Artificial Sequence
  Description of Combined DNA/RNA Molecule Synthetic oligonucleotide
gcuguuaacccuuacugugctt

SEQ ID NO: 87 - DNA, 22 bp, Artificial Sequence
  Description of Combined DNA/RNA Molecule Synthetic oligonucleotide
gcacaguaaggguuaacagctt

SEQ ID NO: 88 - DNA, 22 bp, Artificial Sequence
  Description of Combined DNA/RNA Molecule Synthetic oligonucleotide
uaacccuuacugugcugugctt

SEQ ID NO: 89 - DNA, 22 bp, Artificial Sequence
  Description of Combined DNA/RNA Molecule Synthetic oligonucleotide
gcacagcacaguaaggguuatt

SEQ ID NO: 90 - DNA, 21 bp, Artificial Sequence
  Description of Combined DNA/RNA Molecule Synthetic oligonucleotide
aagcacaacacaguaagggtt

SEQ ID NO: 91 - DNA, 21 bp, Artificial Sequence
  Description of Combined DNA/RNA Molecule Synthetic oligonucleotide
aacccuuacuguguugugctt

SEQ ID NO: 92 - DNA, 21 bp, Artificial Sequence
  Description of Combined DNA/RNA Molecule Synthetic oligonucleotide
acaaugaucugcaugacuctt

SEQ ID NO: 93 - DNA, 21 bp, Artificial Sequence
  Description of Combined DNA/RNA Molecule Synthetic oligonucleotide
aagagucaugcagaucauutt

SEQ ID NO: 94 - RNA, 23 nt, Artificial Sequence
  Description of Artificial Sequence Synthetic oligonucleotide
cagaaacggaugcucccaggccc

SEQ ID NO: 95 - RNA, 23 nt, Artificial Sequence
  Description of Artificial Sequence Synthetic oligonucleotide
gccugggagcauccguuucugcc

SEQ ID NO: 96 - DNA, 23 bp, Artificial Sequence
  Description of Combined DNA/RNA Molecule Synthetic oligonucleotide
cuucgcauuggcucgcaagcatt

SEQ ID NO: 97 - DNA, 23 bp, Artificial Sequence
  Description of Combined DNA/RNA Molecule Synthetic oligonucleotide
ugcuugcgagccaaugcgaagtt

SEQ ID NO: 98 - DNA, 22 bp, Artificial Sequence
  Description of Combined DNA/RNA Molecule Synthetic oligonucleotide
uggucauuuguccugaugcctt

SEQ ID NO: 99 - DNA, 22 bp, Artificial Sequence
  Description of Combined DNA/RNA Molecule Synthetic oligonucleotide
ggcaucaggacaaaugaccatt

SEQ ID NO: 100 - DNA, 23 bp, Artificial Sequence
  Description of Combined DNA/RNA Molecule Synthetic oligonucleotide
ucuucaaggcuggcaucaggatt

SEQ ID NO: 101 - DNA, 23 bp, Artificial Sequence
  Description of Combined DNA/RNA Molecule Synthetic oligonucleotide
uccugaugccagccuugaagatt

SEQ ID NO: 102 - DNA, 23 bp, Artificial Sequence
  Description of Combined DNA/RNA Molecule Synthetic oligonucleotide
auucucagaguagguguaaugtt

SEQ ID NO: 103 - DNA, 23 bp, Artificial Sequence
  Description of Combined DNA/RNA Molecule Synthetic oligonucleotide
cauuacaccuacucugagaautt

SEQ ID NO: 104 - DNA, 21 bp, Artificial Sequence
  Description of Combined DNA/RNA Molecule Synthetic oligonucleotide
gaacacagugagaauggautt

SEQ ID NO: 105 - DNA, 21 bp, Artificial Sequence
  Description of Combined DNA/RNA Molecule Synthetic oligonucleotide
auccauucucacuguguuctt

SEQ ID NO: 106 - DNA, 25 bp, Artificial Sequence
  Description of Artificial Sequence Synthetic oligonucleotide
ggcatcaggacaaatgaccacatca

SEQ ID NO: 107 - DNA, 25 bp, Artificial Sequence
  Description of Artificial Sequence Synthetic oligonucleotide
gacctgcacagatggcggctatcag

SEQ ID NO: 108 - DNA, 23 bp, Artificial Sequence
  Description of Combined DNA/RNA Molecule Synthetic oligonucleotide
gcggauggaguagaacuucggtt

SEQ ID NO: 109 - DNA, 23 bp, Artificial Sequence
  Description of Combined DNA/RNA Molecule Synthetic oligonucleotide
cgcaaguucuacuccauccgctt

SEQ ID NO: 110 - DNA, 23 bp, Artificial Sequence
  Description of Combined DNA/RNA Molecule Synthetic oligonucleotide
caguacugcaccgagaccuugtt

SEQ ID NO: 111 - DNA, 23 bp, Artificial Sequence
  Description of Combined DNA/RNA Molecule Synthetic oligonucleotide
caaggucucggugcaguacugtt

SEQ ID NO: 112 - DNA, 25 bp, Artificial Sequence
  Description of Artificial Sequence Synthetic oligonucleotide
gcggatggagtagaacttgcggtgg

SEQ ID NO: 113 - DNA, 25 bp, Artificial Sequence
  Description of Artificial Sequence Synthetic oligonucleotide
cagtactgcaccgagaccttgcggt

SEQ ID NO: 114 - DNA, 21 bp, Artificial Sequence
  Description of Combined DNA/RNA Molecule Synthetic oligonucleotide
aggagaacccggacuucugtt

SEQ ID NO: 115 - DNA, 21 bp, Artificial Sequence
  Description of Combined DNA/RNA Molecule Synthetic oligonucleotide
cagaaguccggguucuccutt

SEQ ID NO: 116 - DNA, 21 bp, Artificial Sequence
  Description of Combined DNA/RNA Molecule Synthetic oligonucleotide
gagugcagcacgccucgcctt

SEQ ID NO: 117 - DNA, 21 bp, Artificial Sequence
  Description of Combined DNA/RNA Molecule Synthetic oligonucleotide
ggcgaggcgugcugcacuctt

SEQ ID NO: 118 - DNA, 21 bp, Artificial Sequence
  Description of Combined DNA/RNA Molecule Synthetic oligonucleotide
cacugccgaccacucccactt

SEQ ID NO: 119 - DNA, 21 bp, Artificial Sequence
  Description of Combined DNA/RNA Molecule Synthetic oligonucleotide
gtgggaguggucggcagugtt

SEQ ID NO: 120 - DNA, 21 bp, Artificial Sequence
  Description of Combined DNA/RNA Molecule Synthetic oligonucleotide
cagaaacagaugcucccagtt

SEQ ID NO: 121 - DNA, 21 bp, Artificial Sequence
  Description of Combined DNA/RNA Molecule Synthetic oligonucleotide
cugggagcaucuguuucugtt

SEQ ID NO: 122 - DNA, 21 bp, Artificial Sequence
  Description of Combined DNA/RNA Molecule Synthetic oligonucleotide
cagaaaaguaugcucccagtt

SEQ ID NO: 123 - DNA, 21 bp, Artificial Sequence
  Description of Combined DNA/RNA Molecule Synthetic oligonucleotide
cugggagcauacuuuucugtt

SEQ ID NO: 124 - DNA, 14 bp, Artificial Sequence
  Description of Combined DNA/RNA Molecule Synthetic oligonucleotide
aaacggaugcuctt

SEQ ID NO: 125 - DNA, 14 bp, Artificial Sequence
  Description of Combined DNA/RNA Molecule Synthetic oligonucleotide
gagcauccguuutt

SEQ ID NO: 126 - DNA, 16 bp, Artificial Sequence
  Description of Combined DNA/RNA Molecule Synthetic oligonucleotide
gaaacggaugcucctt

SEQ ID NO: 127 - DNA, 16 bp, Artificial Sequence
  Description of Combined DNA/RNA Molecule Synthetic oligonucleotide
ggagcauccguuuctt

SEQ ID NO: 128 - DNA, 18 bp, Artificial Sequence
  Description of Combined DNA/RNA Molecule Synthetic oligonucleotide
agaaacggaugcuccatt

SEQ ID NO: 129 - DNA, 18 bp, Artificial Sequence
  Description of Combined DNA/RNA Molecule Synthetic oligonucleotide
uggagcauccguuucutt

SEQ ID NO: 130 - DNA, 21 bp, Artificial Sequence
  Description of Combined DNA/RNA Molecule Synthetic oligonucleotide
gagaaacggaugcucccactt

SEQ ID NO: 131 - DNA, 21 bp, Artificial Sequence
  Description of Combined DNA/RNA Molecule Synthetic oligonucleotide
gugggagcauccguuucuctt

SEQ ID NO: 132 - DNA, 21 bp, Artificial Sequence
  Description of Combined DNA/RNA Molecule Synthetic oligonucleotide
gugaaacggaugcucccuctt

SEQ ID NO: 133 - DNA, 21 bp, Artificial Sequence
  Description of Combined DNA/RNA Molecule Synthetic oligonucleotide
gagggagcauccguuucactt

SEQ ID NO: 134 - DNA, 21 bp, Artificial Sequence
  Description of Combined DNA/RNA Molecule Synthetic oligonucleotide
gucaaacggaugcucgguctt

SEQ ID NO: 135 - DNA, 21 bp, Artificial Sequence
  Description of Combined DNA/RNA Molecule Synthetic oligonucleotide
gaccgagcauccguuugactt

SEQ ID NO: 136 - DNA, 21 bp, Artificial Sequence
  Description of Combined DNA/RNA Molecule Synthetic oligonucleotide
cagggagcauccguuucagtt

SEQ ID NO: 137 - DNA, 21 bp, Artificial Sequence
  Description of Combined DNA/RNA Molecule Synthetic oligonucleotide
cagggagcauccguuucagtt

SEQ ID NO: 138 - DNA, 21 bp, Artificial Sequence
  Description of Combined DNA/RNA Molecule Synthetic oligonucleotide
cagggugcauccguuucagtt

SEQ ID NO: 139 - DNA, 21 bp, Artificial Sequence
  Description of Combined DNA/RNA Molecule Synthetic oligonucleotide
cagggugcauccguaucagtt

SEQ ID NO: 140 - DNA, 21 bp, Artificial Sequence
  Description of Combined DNA/RNA Molecule Synthetic oligonucleotide
cagggugcaugcguaucagtt

SEQ ID NO: 141 - DNA, 21 bp, Artificial Sequence
  Description of Combined DNA/RNA Molecule Synthetic oligonucleotide
cagggugcuugcguaucagtt

SEQ ID NO: 142 - DNA, 46 bp, Artificial Sequence
  Description of Combined DNA/RNA Molecule Synthetic oligonucleotide
aggagaacccggacuucuguuuuuucagaaguccggguucuccutt

SEQ ID NO: 143 - DNA, 46 bp, Artificial Sequence
  Description of Combined DNA/RNA Molecule Synthetic oligonucleotide
gagugcagcacgccucgccuuuuuuggcgaggcgugcugcacuctt

SEQ ID NO: 144 - DNA, 45 bp, Artificial Sequence
  Description of Combined DNA/RNA Molecule Synthetic oligonucleotide
aggagaacccggacuucuguuuuuucagaaguccggguucuccut

SEQ ID NO: 145 - DNA, 43 bp, Artificial Sequence
  Description of Combined DNA/RNA Molecule Synthetic oligonucleotide
aggagaacccggacuucuguucgcagaaguccggguucuccut

SEQ ID NO: 146 - DNA, 43 bp, Artificial Sequence; identical to SEQ ID NO: 145
  Description of Combined DNA/RNA Molecule Synthetic oligonucleotide

SEQ ID NO: 147 - DNA, 21 bp, Artificial Sequence
  Description of Combined DNA/RNA Molecule Synthetic oligonucleotide
aguaacccuuacugugcuatt

SEQ ID NO: 148 - DNA, 21 bp, Artificial Sequence
  Description of Combined DNA/RNA Molecule Synthetic oligonucleotide
uagcacaguaaggguuacutt

SEQ ID NO: 149 - DNA, 21 bp, Artificial Sequence
  Description of Combined DNA/RNA Molecule Synthetic oligonucleotide
agauaacccuuacugugcutt

SEQ ID NO: 150 - DNA, 21 bp, Artificial Sequence
  Description of Combined DNA/RNA Molecule Synthetic oligonucleotide
agcacaguaaggguuaucutt

SEQ ID NO: 151 - DNA, 24 bp, Artificial Sequence
  Description of Artificial Sequence Synthetic oligonucleotide
ttaacccttactgtgctgtgctgt